(19) United States
(12) Patent Application Publication
(10) Pub. No.: US 2016/0282364 A1
(43) Pub. Date: Sep. 29, 2016

(54) ASSAY FOR INSULIN-DEGRADING ENZYME (IDE) INHIBITORS

(71) Applicant: PRESIDENT AND FELLOWS OF HARVARD COLLEGE, Cambridge, MA (US)

(72) Inventors: Juan Pablo Maianti, Revere, MA (US); Amanda McFedries, Lowell, MA (US); Ralph E. Kleiner, New York, NY (US); Alan Saghatelian, Somerville, MA (US); David R. Liu, Lexington, MA (US)

(73) Assignee: President and Fellows of Harvard College, Cambridge, MA (US)

(21) Appl. No.: 15/034,731

(22) PCT Filed: Nov. 6, 2014
(86) PCT No.: PCT/US2014/064322

§ 371 Date: May 5, 2016

Related U.S. Application Data
(60) Provisional application No. 61/900,789, filed on Nov. 6, 2013.

(51) Int. Cl. G01N 33/68; C07K 5/087; C07K 5/09; C07K 5/083; C12Q 1/68; G01N 33/58

(52) U.S. Cl. CPC G01N 33/6893; C12Q 1/6804; G01N 33/582; C07K 5/0815; C07K 5/0817; C07K 5/0808; C07K 5/0812; C12Y 304/24056; G01N 2333/96486; A61K 38/00

(57) ABSTRACT

IDE-binding probes and assays for the identification of IDE-binding and IDE-inhibiting compounds are provided. Pharmaceutical compositions of macrocyclic IDE inhibitors are also provided, including compositions in which such IDE inhibitors are combined with an additional therapeutic agent. Methods of using IDE inhibitors for transiently inhibiting IDE in a subject in need thereof, for example, for the transient inhibition of IDE in a subject exhibiting aberrant IDE activity, impaired isulin signaling, or insulin resistance, for example, a subject having diabetes, are also provided. Methods of using IDE inhibitors for transiently modulating heart rate and/or blood pressure in a subject are also provided.

### RELATED APPLICATION

This application claims priority under 35 U.S.C. §119(e) to U.S. provisional patent application, U.S. Ser. No. 61/900,789, filed Nov. 6, 2013, which is incorporated herein by reference.

### GOVERNMENT SUPPORT

This invention was made with Government support under grant R01 GM065865 awarded by the National Institutes of Health. The Government has certain rights in this invention.

### BACKGROUND OF THE INVENTION

Research into the biosynthesis, secretion, and signaling of insulin has led to the development of important treatments for diabetes. In contrast, despite 60 years of speculation that inhibiting the degradation of insulin could treat diabetes, and the identification of insulin-degrading enzyme (IDE) as a diabetes susceptibility gene, the relationship between IDE activity and glucose homeostasis remains unclear due to the lack of IDE inhibitors that are active in vivo.

Insulin-degrading enzyme, also sometimes referred to as insulysin or insulin protease, is a 110 kDa zinc-binding protease of the M16A metalloprotease subfamily. IDE was first identified by its ability to degrade the β chain of insulin and has since been shown to target additional substrates, including the pathophysiologically important peptide β-amyloid, and the signaling peptides glucagon, TGF-alpha, β-endorphin, and atrial natriuric peptide. While IDE is the main protease responsible for insulin degradation, most other IDE substrates are known to be targeted and degraded by other proteases as well, and the effect of IDE inhibition on such substrates has not been delineated.

Despite great interest in pharmacological targeting of IDE, the enzyme has remained an elusive target. The only known series of IDE-targeted inhibitors to date are peptide hydroxamic acids, e.g., Ii1 (Inhibitor of IDE1, see FIG. 1e and, e.g., Leissring et al. (2010), Designed Inhibitors of Insulin-Degrading Enzyme Regulate the Catabolism and Activity of Insulin. PLoS ONE 5(5): e10504); and macrocyclic IDE inhibitors as described in international PCT application, PCT/US2012/044977, filed Jun. 29, 2012, entitled “Macrocyclic Insulin-Degrading Enzyme (IDE) Inhibitors and Uses Thereof,” and published under Publication No. WO/2013/006451 on Jan. 10, 2013. See also Bannister T D et al., ML345: A Small-Molecule Inhibitor of the Insulin-Degrading Enzyme (IDE); 2012; In: Probe Reports from the NIH Molecular Libraries Program; Bethesda (Md.): National Center for Biotechnology Information (US); and Abdul-Hay et al., J Med Chem 2013.

One important application for IDE inhibitors is the treatment of diabetes, a group of endocrinological disorders that are characterized by impaired insulin signaling or insulin resistance. Conventional therapeutic approaches for diabetic patients aim to enhance insulin signaling, for example, by administration of exogenous insulin, by stimulating the generation and secretion of endogenous insulin, or by activating downstream targets of the insulin receptor (IR) signaling cascade. IDE inhibitors open another therapeutic avenue to improve insulin signaling by inhibiting IDE-mediated insulin catabolism.

### SUMMARY OF THE INVENTION

Some aspects of this disclosure are based on the discovery and characterization of the first potent, selective, and physiologically active IDE inhibitor, which was identified from a DNA-templated macrocycle library. A co-crystal structure of the inhibitor bound to IDE reveals that this macrocycle engages a novel binding site that explains its remarkable selectivity for IDE. Some aspects of this disclosure are based on the discovery that IDE-binding and IDE-inhibiting compounds can be identified by using competitive binding assays in which a candidate compound competes with a probe comprising a known IDE-binding molecule, e.g., an IDE inhibitor as described herein such as compound 6bk.

Some aspects of this disclosure are based on the surprising discovery from the treatment of lean and obese mice with IDE inhibitors under a variety of different conditions that IDE regulates the abundance and signaling of glucagon and amylin, in addition to that of insulin. Some aspects of this disclosure are based on the surprising discovery that, under physiologic conditions that augment insulin and amylin levels, such as oral glucose administration, acute IDE inhibition can lead to substantially improved glucose tolerance and slower gastric emptying. In addition, some aspects of this disclosure are based on the surprising discovery that acute IDE inhibition can modulate the effects of Calcitonin Gene-Related Peptide (CGRP) signaling, e.g., augment CGRP-induced blood glucose level fluctuations, and reduce CGRP-induced blood pressure and heart rate increases. It was also discovered that acute IDE inhibition can lower baseline blood pressure and heart rate. These discoveries transform the previous understanding of IDE's roles in glucose regulation and demonstrate a potential therapeutic strategy of modulating IDE activity to treat type-2 diabetes.

Some aspects of this disclosure provide methods for identifying insulin-degrading enzyme (IDE)-binding compounds. Such methods are useful for the identification of novel IDE modulators, e.g., inhibitors with improved IDE-binding properties as compared to currently known IDE inhibitors, and IDE activators. In general, the methods comprise contacting an IDE with a probe that binds IDE with an IC₅₀ of 10 μM or less, wherein the probe comprises a detectable label, and with a candidate compound under conditions suitable for the probe and the candidate compound to bind the IDE; determining the level of unbound probe in the presence of the candidate compound; and comparing the level of unbound probe to a reference level, wherein if the level of unbound probe in the presence of the candidate compound is higher than the reference level, then the candidate compound is identified as an IDE-binding compound. In some embodiments, the IDE-binding probe comprises a macrocyclic IDE-binding molecule. In some embodiments, the macrocyclic IDE-binding molecule comprises a compound of any of Formula (I)-(VI). In some embodiments, the macrocyclic IDE-binding molecule comprises a structure selected from the group consisting of structures 1b, 2b, 3b, 4b, 5b, 6a, 6c, 6b, 6bk, and 7-29. In some embodiments, the macrocyclic IDE-binding molecule comprises structure 6bk. In some embodiments, the macrocyclic IDE-binding molecule is conjugated to the detectable label. In some embodiments, the macrocyclic IDE-binding molecule is conjugated to the detectable label via a linker. In some embodiments, the detectable label comprises a fluorophore. In some embodiments, the probe is compound 31.

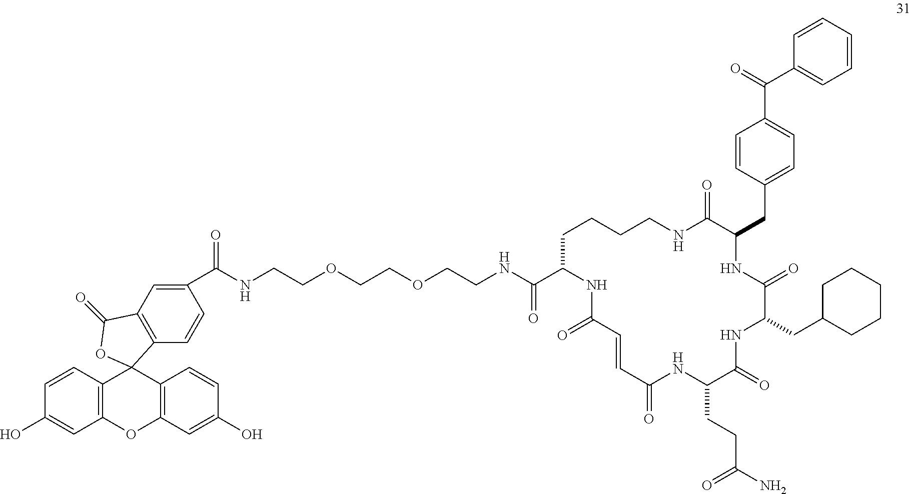 [SMILES: NC(=O)CC[C@@H]1NC(=O)/C=C/C(=O)N[C@H](C(=O)NCCOCCOCCNC(=O)c2ccc3c(c2)C(=O)OC32c3ccc(O)cc3Oc3cc(O)ccc32)CCCCNC(=O)[C@@H](Cc2ccc(C(=O)c3ccccc3)cc2)NC(=O)[C@H](CC2CCCCC2)NC1=O]

In some embodiments, determining the level of unbound probe in the presence of the candidate compound comprises exposing IDE contacted with the probe and the candidate compound to incident, plane-polarized light of a suitable wave length to excite the fluorophore; and detecting the level of fluorescent light emitted by the fluorophore in the same plane of polarization as the incident light, as well as the level of fluorescent light emitted by the fluorophore in a plane different from the plane of polarization of the incident light. In some embodiments, determining the level of unbound probe in the presence of the candidate compound comprises calculating the level of unbound probe from the levels of emitted light detected. In some embodiments, the calculating the level of unbound probe comprises calculating a ratio of the levels of emitted light detected, or calculating a fluorescence anisotropy value.

In some embodiments, the candidate compound is identified as an IDE-binding compound if the level of fluorescent light emitted by the fluorophore in the presence of the candidate compound in a plane different from the plane of polarization of the incident light is higher than a reference level of fluorescent light emitted in that plane measured in the absence of the candidate compound. In some embodiments, the method is carried out repeatedly for a candidate compound at a plurality of IDE concentrations, and the method comprises calculating a ratio of the levels of emitted light detected, or calculating a fluorescence anisotropy value for each concentration; and determining a dynamic IDE concentration range. In some embodiments, the candidate compound is identified as an IDE-binding compound if the level of fluorescent light emitted by the fluorophore in the presence of the candidate compound in a plane different from the plane of polarization of the incident light is at least 1.1-fold, at least 1.2-fold, at least 1.3-fold, at least 1.5-fold, at least 1.75-fold, at least 2-fold, at least 2.5-fold, at least 3-fold, at least 4-fold, at least 5-fold, at least 10-fold, at least 20-fold, at least 30-fold, at least 40-fold, at least 50-fold, at least 100-fold, at least 200-fold, at least 500-fold, or at least 1000-fold higher than a reference level of fluorescent light emitted in that plane measured in the absence of the candidate compound. In some embodiments, the candidate compound is identified as an IDE-binding compound if the fluorescence anisotropy in the presence of the candidate compound is at least 1.1-fold to at least 5-fold, at least 10-fold to at least 100-fold, or at least 200-fold to at least 1000-fold lower than the fluorescence anisotropy in the absence of the candidate compound. In some embodiments, the level of emitted light or the fluorescent anisotropy is measured at a point within the dynamic IDE concentration range.

In some embodiments, the detectable label comprises a binding agent. In some embodiments, the binding agent comprises an antibody or an antigen-binding fragment thereof. In some embodiments, the binding agent comprises a ligand. In some embodiments, the ligand is biotin or an avidin derivative. In some embodiments, the probe comprises compound 30. In some embodiments, the detectable label comprises a detectable isotope.

In some embodiments, IDE is contacted with the probe and the candidate compound in aqueous solution. In some embodiments, the IDE is contacted with the probe and the candidate compound under physiological conditions. In some embodiments, the method comprises screening a library of different candidate compounds. In some embodiments, the reference level represents a level of unbound probe in the absence of the candidate compound. In some embodiments, the reference level is determined by measuring the level of unbound probe in the absence of a candidate compound or in the presence of a compound known to bind IDE with an IC₅₀ of more than 10 μM. In some embodiments, the probe comprises an IDE inhibitor, and the candidate compound is identified as an IDE inhibitor if it can successfully compete with the probe for IDE binding.

Some aspects of this disclosure provide IDE-binding compounds that are conjugated to a detectable label. Such compounds are useful as probes in the methods for identifying IDE-binding compounds described herein. In some embodiments, a compound is provided as described by Formula (V):

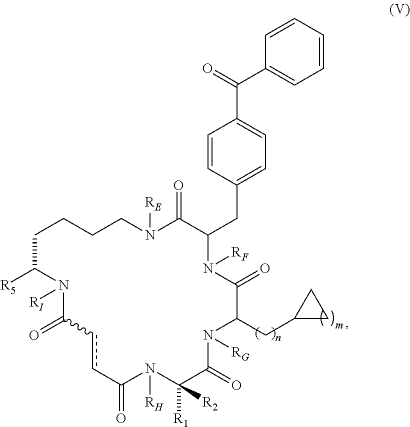

or a pharmaceutically acceptable salt, solvate, hydrate, stereoisomer, polymorph, tautomer, isotopically enriched form, or prodrug thereof, wherein R5 comprises the detectable label.

In some embodiments, the compound comprises a structure selected from the group consisting of structures 1b, 2b, 3b, 4b, 5b, 6a, 6c, 6b, 6bk and 7-31. In some embodiments, the compound comprises 6bk. In some embodiments, the detectable label comprises a fluorophore. In some embodiments, the compound is of Formula 31.

Some aspects of this disclosure provide compositions comprising a macrocyclic IDE inhibitor as described herein and an IDE-binding molecule. In some embodiments, the IDE-binding molecule is an IDE inhibitor. In some embodiments, the IDE-binding molecule is an IDE-substrate. In some embodiments, the IDE substrate is insulin, amylin, or glucagon. Some aspects of this disclosure provide compositions comprising an IDE, a macrocyclic compound conjugated to a detectable label, e.g., a compound comprising a structure selected from the group consisting of structures 1b, 2b, 3b, 4b, 5b, 6a, 6c, 6b, 6bk, and 7-31, and a candidate IDE modulator (e.g., a candidate IDE inhibiting or activating compound).

Some aspects of this disclosure provide pharmaceutical compositions comprising an IDE-binding compound as described herein, or as identified by the methods provided herein, or a pharmaceutically acceptable salt, solvate, hydrate, stereoisomer, polymorph, tautomer, isotopically enriched form, or prodrug thereof, in an amount effective to inhibit IDE in a subject, and, optionally a pharmaceutically acceptable carrier.

Some aspects of this disclosure provide methods comprising administering an IDE inhibitor, e.g., an IDE inhibitor provided herein or identified by the methods provided herein, to a subject in an amount effective to inhibit IDE activity in the subject. In some embodiments, the IDE inhibitor is an IDE inhibitor described herein, e.g., in any of Formulae (I)-(VIII), 1b, 2b, 3b, 4b, 5b, 6a, 6b, 6bK, 6c, or 1-31, or an IDE-inhibitor as described in international PCT application, PCT/US2012/044977, entitled “Macrocyclic Insulin-Degrading Enzyme (IDE) Inhibitors and Uses Thereof,” filed Jun. 29, 2012, and published under Publication No. WO/2013/006451 on Jan. 10, 2013, the entire contents of which are incorporated herein by reference. In some embodiments, the IDE inhibitor is administered in an amount effective to modulate the stability and/or signaling of glucagon and/or amylin in the subject. For example, the IDE inhibitor may be administered according to a dosing schedule resulting in transient IDE inhibition. In some embodiments, the transient IDE inhibition subsides before an upregulation of glucagon signaling in the subject occurs. In some embodiments, IDE activity is reduced in the subject to less than 75%, less than 60%, less than 50%, less than 40%, less than 30%, less than 25%, less than 20%, or less than 10% of IDE activity in the subject in the absence of the IDE inhibitor. In some embodiments, the transient IDE inhibition is for less than 1 hour, less than 2 hours, less than 3 hours, less than 4 hours, less than 5 hours, or less than 6 hours. In some embodiments, the IDE inhibitor is administered in temporal proximity to the subject eating a meal. In some embodiments, the IDE inhibitor is administered within 1 hour before the meal, immediately before the meal, during the meal, immediately after the meal, or within 1 hour after the meal. In some embodiments, the IDE inhibitor is administered according to a dosing schedule that results in a return of IDE activity to pre-administration levels within an hour before or after blood glucose concentration returns to baseline levels in the subject after a meal.

In some embodiments, the IDE inhibitor is of Formula VII or VIII:

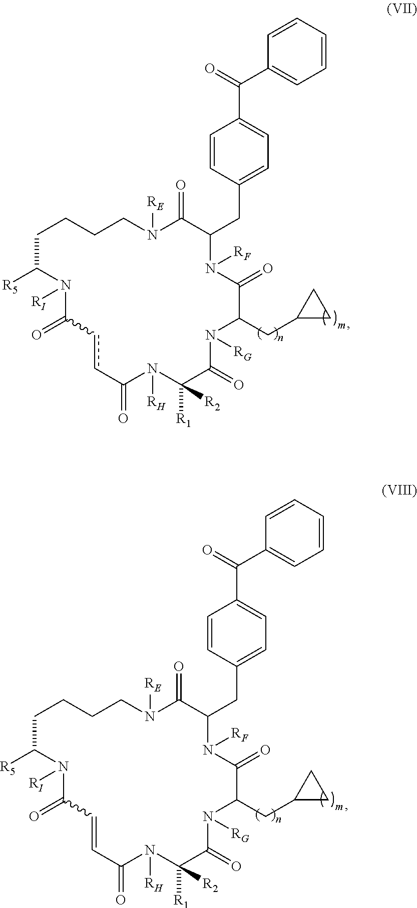

or a pharmaceutically acceptable salt, solvate, hydrate, stereoisomer, polymorph, tautomer, isotopically enriched form, or prodrug thereof.

In some embodiments, the compound is any one of compounds 1b, 2b, 3b, 4b, 5b, 6a, 6b, 6c, or 7-31.

In some embodiments, methods are provided that comprise determining the level of IDE activity in the subject, for example, by obtaining a biological sample comprising IDE from the subject, such as, for example, a body fluid, cell, or tissue sample, and contacting the IDE with a probe, e.g., an IDE-binding compound conjugated to a detectable label as provided herein, e.g., a probe comprising the structure of any one of compounds 1b, 2b, 3b, 4b, 5b, 6a, 6b, 6c, or 7-31, and detecting the level of binding of the probe to IDE, e.g., by a method described herein. Such methods are useful to detect abnormalities in IDE abundance and/or IDE binding, e.g., IDE hypo- or hyperactivity, which are associated with pathological states of insulin signaling and glucose homoeostasis. In some embodiments, such methods further comprises determining the level of blood glucose or of insulin in the subject. In some embodiments, the method further comprises determining the stability and/or the level of signaling of glucagon in the subject. In some embodiments, the method further comprises determining the stability and/or the level of signaling of amylin in the subject. In some embodiments, the method further comprises adjusting the administration schedule of the IDE inhibitor based on the determined level of IDE activity; glucagon stability and/or signaling; or amylin stability and/or signaling; in order to achieve a desired level of activity, stability, or signaling, e.g., a level found in a healthy subject. In some embodiments, the subject has been diagnosed with diabetes or pre-diabetes.

Some aspects of this disclosure provide kits comprising a dosage form of an IDE inhibitor of Formula (VII) or (VIII); and instructions for administering the IDE inhibitor to achieve transient IDE inhibition.

Some aspects of this disclosure provide kits for comprising an IDE-binding probe comprising an IDE-inhibitor conjugated to a detectable label; and instructions for performing an assay for identifying an IDE-binding compound.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIG. 1. Discovery of potent and highly selective macrocyclic IDE inhibitors from the in vitro selection of a DNA-templated macrocycle library. A. Structure of the most potent hit from the IDE selection (6b) and a summary of the requirements for IDE inhibition revealed by the synthesis and evaluation of 6b analogs. B. IDE inhibition potency of selection hits 1b to 6b and 30 structurally related analogs in which the linker, scaffold, and three building blocks were systematically varied. C. Structure of physiologically active IDE inhibitor 6bK. D. Structure of the inactive diastereomer bisepi-6bK. E. Structure of the previously reported substrate-mimetic hydroxamic acid inhibitor Ii1¹². F. Selectivity analysis of macrocycle 6bK reveals >1,000-fold selectivity for IDE (IC₅₀=50 nM) over all other metalloproteases tested. G. Inhibitor Ii1¹² inhibits IDE (IDE, IC₅₀=0.6 nM), and also thimet oligopeptidase (THOP, IC₅₀=6 nM) and neurolysin (NLN, IC₅₀=185 nM), but not neprilysin (NEP), matrix metalloprotease 1 (MMP1), or angiotensin converting-enzyme (ACE). Human IDE shares 95% primary sequence homology with mouse IDE²², and both are inhibited by the macrocycles used in this study with similar potency (Data FIG. 8).

FIG. 2. Structural basis of IDE inhibition by macrocycle 6b. A. X-ray co-crystal structure of IDE bound to macrocyclic inhibitor 6b (2.7 Å resolution, pdb: 4LTE). IDE domains 1, 2, 3, and 4 are colored green, blue, yellow, and red, respectively. Macrocycle 6b is represented as a ball-and-stick model, and the catalytic zinc atom is represented as an orange sphere. B. Electron density map (composite omit map contoured at 1σ) and model of IDE-bound macrocycle 6b interacting with a 10 Å-deep hydrophobic pocket. C. Relative position of macrocycle 6b bound 11 Å from the catalytic zinc atom. D. Overlay of the 6b model (surface rendering) on the IDE:insulin co-crystal structure (pdb: 2WBY)²⁴. E. Activity assays for wild-type or mutant human IDE variants in the presence of 6bK. Mutagenesis of residues Ala479Leu (▴) and Gly362Gln () hindered the inhibition potency of 6bK by >600- and >50-fold, respectively, compared to that of wild-type human IDE.

FIG. 3. Physiological consequences of acute IDE inhibition by 6bK on glucose tolerance in lean and DIO mice. A and B. Oral glucose tolerance during acute IDE inhibition. A. Male C57BL/6J lean (25 g) mice were treated with a single i.p. injection of IDE inhibitor 6bK (80 mg/kg), inactive control bisepi-6bK (80 mg/kg), or vehicle alone (30 min prior to glucose gavage (3.0 g/kg). B. DIO mice (35-45 g) were treated with 6bK (60 mg/kg), and inactive control bisepi-6bK (60 mg/kg) or vehicle alone 30 min prior to glucose gavage (3.0 g/kg). C and D. Glucose tolerance phenotypes after i.p. injection of glucose (1.5 g/kg) in lean (c) and DIO (d) male mice treated with 6K, inactive bisepi-6bK, or vehicle alone. Area under the curve (AUC) calculations are shown in FIG. 14. All data points and error bars represent mean±SEM. Significance tests were performed using two-tail Student's t-test, and significance levels shown are p<0.05 (*) or p<0.01 (**) versus the vehicle-only control group.

FIG. 4. Acute IDE inhibition affects the abundance of multiple hormone substrates and their corresponding effects on blood glucose levels. A. Plasma hormone measurements at 20 and 135 minutes post-IPGTT (FIG. 3d) for DIO mice treated with 6bK or vehicle alone. RT-PCR analysis of DIO liver samples collected at 135 min post-IPGTT reveals 50% higher glucose-6-phosphatease (G6Pase) and 30% lower phosphoenolpyruvate carboxykinase (PEPCK) transcript levels for the 6bK-treated cohort versus vehicle-only (V) controls. B to D, Blood glucose responses and abundance of injected hormones in lean mice 30 min after treatment with 6bK (80 mg/kg) or vehicle alone. B. Insulin s.c. (0.25 U/kg) after 5-hour fast. C. Amylin s.c. (250 μg/kg) after overnight fast. D. Glucagon s.c. (100 μg/kg) after overnight fast. Trunk blood was collected at the last time points for plasma hormone measurements (insets). All data points and error bars represent mean±SEM. Significance tests were performed using two-tail Student's t-test, and significance levels shown are p<0.05 (*) or p<0.01 (**) versus the vehicle-only control group.

FIG. 5. Acute IDE inhibition modulates the endogenous signaling activity of glucagon, amylin and insulin. A and B. G-protein-coupled glucagon receptor knockout mice (GCGR¹, C57BL/6J background) treated with IDE inhibitor 6bK (80 mg/kg) display altered glucose tolerance relative to vehicle-treated mice if challenged with oral glucose (a) but not i.p. injected glucose (b). C. Wild-type mice fasted overnight were injected i.p. with pyruvate (2.0 g/kg) 30 min after treatment with 6bK, inactive analog bisepi-6bK, or vehicle alone. D. Plasma hormone measurements 60 min post-PTT reveal elevated glucagon but similar insulin levels for the 6bK-treated cohort relative to bisepi-6bK, or vehicle controls. E. RT-PCR analysis of liver samples 60-min post-PTT revealed elevated gluconeogenesis transcriptional markers for the 6bK-treated group relative to vehicle controls. F. Acute IDE inhibition slows gastric emptying through amylin signaling. Wild-type mice fasted overnight were given an oral glucose bolus (3.0 g/kg supplemented with 0.1 mg/mL phenol red) 30 min after treatment with 6bK alone, 6bK co-administered with the specific amylin receptor antagonist AC187 (3 mg/kg i.p.), vehicle alone, or inactive bisepi-6bK. The stomachs were dissected at 30 min post-glucose gavage. All data points and error bars represent mean±SEM. Significance tests were performed using two-tail Student's t-test, and significance levels shown are p<0.05 (*) or p<0.01 (**) versus the vehicle-only control group.

FIG. 6. Model for the expanded roles of IDE in glucose homeostasis and gastric emptying based on the results described herein. IDE inhibition increases the abundance and signaling of three key pancreatic peptidic hormones, insulin, amylin, and glucagon, with the corresponding physiological effects shown in blue and red.

FIG. 7. A. Overview of the in vitro selection of a 13,824-membered DNA-templated macrocycle library for IDE binding affinityl^{15,16}. B. Enrichment results from two independent in vitro selections against N-His₆-mIDE using the DNA-templated macrocycle library¹⁵. The numbers highlight compounds enriched at least 2-fold in both selections. C. Structures of IDE-binding macrocycles 1-6 decoded from DNA library barcodes corresponding to building blocks A, B, C and D. The cis and trans isomers are labeled ‘a’ and ‘b’, respectively. The two isomers were synthesized as previously reported^{15,16} and separated by HPLC. IDE inhibition activity was assayed by following cleavage of the fluorogenic peptide substrate Mca-RPPGFSAFK(Dnp)-OH.

FIG. 8. Inhibition of human and mouse IDE activity demonstrated using distinct assays. A and B. cleavage of the fluorogenic substrate peptide Mca-RPPGFSAFK(Dnp)-OH by human and mouse IDE in the presence of inhibitors (A) 6b and (B) 6bK. C. Homogeneous time-resolved fluorescence (HTRF, Cisbio) assay measuring degradation of insulin by IDE (R&D) in the presence of 6b, 6bK and 28. D. LC-MS assay for ex vivo degradation of CGRP (10 μM) by endogenous IDE in mouse plasma in the presence of 6b. E and F. Biochemical assays suggesting that 6b binds a site in IDE distinct from the conventional peptide substrate binding site known to bind substrate mimetic Ii1. Yonetani-Theorell double inhibitor plots of IDE activity in the presence of (E) 6b and Ii1, or (F) 6b and bacitracin. Crossing lines indicate synergistic and independent binding of inhibitors, while parallel lines indicate competition for binding to the enzyme. The inhibitors assayed using the fluorogenic substrate Mca-RPPGFSAFK(Dnp)-OH.

FIG. 9. Molecular docking simulation of 6b, and fluorescence polarization measurements with fluorescein-labeled 6b analog 31. A. Insulin competes with the fluorescein-labeled macrocycle inhibitor (31) for binding IDE. Cysteine-free IDE was titrated to 0.9 nM of macrocycle 31 in the absence or presence of 2.5 μM insulin. Representative fluorescence polarization plots are shown. Dissociation constants (K_{D}) were calculated from individual runs and averaged and shown with fitting error-weighted standard deviations. B. Molecular docking simulations are consistent with the placement of building blocks A and B in the structural model. The structure of 6b in binding site from crystallographic data with composite omit map contoured at 1.0σ (p-benzoyl-phenylalanine is shown in red, cyclohexylalanine in blue, and the backbone in purple). C. Highest-scoring pose from DOCK simulations (glutamine group is shown in green). D. Residue decomposed energy of the crystal (green) and docked (blue) poses of 6b.

FIG. 10. Small molecule-enzyme mutant complementation study to confirm the macrocycle binding site and placement of the benzophenone and cyclohexyl building-block groups. A. IDE mutant A479L is inhibited by 6b>600-fold less potently compared to wild-type IDE. B. Analog 9, in which the p-benzoyl ring is substituted for a smaller tert-butyl group, inhibits A479L IDE and WT IDE comparably. C. Similarly, IDE mutant G362Q is inhibited 77-fold less potently by 6b compared with WT IDE. D. Analog 13, in which the L-cyclohexyl alanine side chain was substituted with a smaller L-leucine side chain, inhibits G362Q IDE and WT IDE comparably. The full list of IDE mutants investigated is shown in Table 7.

FIG. 11. Pharmacokinetic parameters of 6bK in a physiologically active and well tolerated dose. A. Plasma binding, plasma stability, and microsomal stability (1 h incubation) data was provided by Dr. Stephen Johnston and Dr. Carrie Mosher (Broad Institute). B. Heavy 6bK was synthesized with ¹⁵N,¹³C-lysine for stable-isotope dilution LC-MS quantitation. C. Concentration of 6bK in mice tissues and plasma collected over 4 hours (n=1-2). D. Average biodistribution of 6bK in five lean mice at 150 min post-injection of 6bK 80 mg/kg i.p. at the endpoint of a IPGTT experiment. We did not detect 6bK in the brain even using 10-fold concentrated samples for LC-MS injection compared to other tissues. E. Increased hypoglycemic response to a high dose of insulin (1.0 U/kg) under acute IDE inhibition (compare with FIG. 4b). Non-fasted male C57BL/6J mice were treated with a single i.p. injection of IDE inhibitor 6bK (80 mg/kg) or vehicle alone, followed by i.p. insulin (Humulin-R, 1.0 U/kg). The animals in the 6bK-treated cohort experienced lethargy and hypothermia, and were sacrificed at 75 min post-insulin injection. F. Body weight measurements for C57BL/6J mice dosed 6bK (80 mg/kg i.p.) or vehicle alone. All data points and error bars represent mean±SEM. Statistics were performed using a two-tail Student's t-test, and significance levels shown in the figures are *p<0.05 versus vehicle control group or **p<0.01 versus vehicle control group.

FIG. 12. Dependence of insulin and glucagon secretion on the route of glucose administration (oral or i.p.) due to the both the ‘incretin effect’ as well as the hyperinsulinemic phenotype of DIO versus lean mice. A. The early insulin response to glucose in lean and DIO mice is higher during OGTT than IPGTT. B. Suppression of glucagon secretion post-glucose administration is less effective after IPGTT and in DIO mice. All data points and error bars represent mean±SEM. Statistics were performed using a two-tail Student's t-test, and significance levels shown in the figures are *p<0.05 or **p<0.01 between the labeled groups.

FIG. 13. Low-potency diastereomers of 6bK used to determine effective dose range of 2 mg/mouse and confirm on-target IDE inhibition effects during IPGTTs in lean and DIO mice. A. Inhibition of mouse IDE activity by low potency diastereomers of 6bK. The stereocenters altered in each compound relative to those of 6bK are labeled with a star. B. The effects of 6bK (90 mg/kg) were compared to the weakly active stereoisomer epi-C-6bK (90 mg/kg) and vehicle controls during an IPGTT to determine the dosing range in lean mice. C. Two doses of 6bK, 80 mg/kg and 40 mg/kg, were compared with inactive bisepi-6bK (80 mg/kg) and vehicle alone. D and E. Administration of 6bK (80 mg/kg) to lean mice not followed by injection of a nutrient such as glucose or pyruvate did not significantly alter (D) basal blood glucose or (E) basal hormone levels compared to bisepi-C6bK or vehicle controls. All data points and error bars represent mean±SEM. Statistics were performed using a two-tail Student's t-test, and significance levels shown in the figures are *p<0.05 versus vehicle control group; **p<0.01 versus vehicle control group.

FIG. 14. Relative area under the curve calculations and 6bK dose response for the glucose tolerance tests shown in FIG. 3. A. During OGTT, lean and DIO mice treated with 6bK (2 mg/animal) display improved glucose tolerance (lower blood glucose area), compared to vehicle controls and inactive bisepi-6bK. B. In contrast, during IPGTT both lean and DIO mice treated with 6bK display impaired glucose tolerance (higher blood glucose area) compared to vehicle or bisepi-6bK controls. C and D. Dose-response of 6bK (40 and 90 mg/kg; see FIG. 3d for 60 mg/kg) followed by IPGTT in DIO mice. Vehicle alone and a matching dose of bisepi-6bK were used as controls. C. DIO mice treated with low doses of 6bK (40 mg/kg) responded to IPGTT in either of two ways: improved glucose tolerance throughout the experiment (n=3) or a hyperglycemic rebound as described in the main text (n=4), suggesting this dose is too low to achieve a consistent effect (note the large error bars). D. Mice treated with high doses of 6bK (90 mg/kg) respond similarly to 60 mg/kg (FIG. 3d), but the potential weak activity observed for bisepi-6bK (IC₅₀>100 μM) at this high dose compared to vehicle alone suggests that 90 mg/kg may be too high. All data points and error bars represent mean±SEM. Statistics were performed using a two-tail Student's t-test, and significance levels shown in the figures is *p<0.05 versus vehicle control group.

FIG. 15. ¹H- and ¹³C-NMR spectra of 6b, 6bK, and bisepi-6bK.

FIG. 16. Dose-response of CGRP i.v. bolus (single mouse).

FIG. 17. Representative blood pressure (BP) data.

FIG. 18. 6bK affects BP baseline and increases CGRP response duration.

FIG. 19. 6bK lowers baseline heart rate in connection to baseline BP.

FIG. 20. 6bK augments CGRP-induced blood glucose excursions.

### DEFINITIONS

### Chemical Definitions

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The entire contents of each references cited in this paragraph are incorporated by reference.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions, p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

Where an isomer/enantiomer is preferred, it may, in some embodiments, be provided substantially free of the corresponding enantiomer and may also be referred to as “optically enriched.” “Optically enriched,” as used herein, means that the compound is made up of a significantly greater proportion of one enantiomer. In certain embodiments, the compound of the present invention is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); Wilen, Tables of Resolving Agents and Optical Resolutions, p. 268 (E. L. Eliel, Ed., Univ. of Notre Dame Press, Notre Dame, Ind. 1972).

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C_{5 6} alkyl.

The definitions of chemical terms as set forth in the “Chemical Definitions” section, paragraphs [0039]-[0083], on pages 21-42 of international PCT application PCT/US2012/044977, filed Jun. 29, 2012, entitled “Macrocyclic Insulin-Degrading Enzyme (IDE) Inhibitors and Uses Thereof,” as published under Publication No. WO/2013/006451 on Jan. 10, 2013, are incorporated herein in their entirety by reference. The definitions set forth in the “Chemical Definitions” section of that application shall govern the terms used herein. In case of a conflict, the instant specification shall control. Some of the exemplary substituents and groups described in that section, and additional substituents and groups, are described in more detail in the Detailed Description, Examples, Figures, and Claims herein. The disclosure is not meant to be limited by the exemplary listing of substituents and groups in this application or in Publication No. WO/2013/006451.

### Other Definitions

As used herein, the term “pharmaceutically acceptable salt” refers to those salts which are, within the scope of sound medical judgment, suitable for use in humans and other animals without undue toxicity, irritation, and immunological response, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate, and aryl sulfonate.

A “subject” to which administration is contemplated includes, but is not limited to, humans (i.e., a male or female of any age group, e.g., a pediatric subject (e.g, infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs), birds (e.g., commercially relevant birds such as chickens, ducks, geese, and/or turkeys), reptiles, amphibians, and fish. In certain embodiments, the non-human animal is a mammal. The non-human animal may be a male or female at any stage of development. A non-human animal may be a transgenic animal.

The terms “administer,” “administering,” or “administration,” as used herein, refer to implanting, absorbing, ingesting, injecting, or inhaling a substance, for example, a compound or composition as described herein.

The terms “conjugating,” “conjugated,” and “conjugation” refer to an association of two entities, for example, of two molecules such as an IDE inhibitor and a fluorophore. The association can be, for example, via a direct or indirect (e.g., via a linker) covalent linkage or via non-covalent interactions. In some embodiments, the association is covalent. In some embodiments, the association is via a covalent bond. In some embodiments, two molecules are conjugated via a linker connecting both molecules.

The term “detectable label” refers to a moiety that comprises at least one element, isotope, or functional group that enables or facilitates detection of a molecule, e.g., an IDE-binding molecule, to which the label is attached. Labels can be directly attached (e.g., via a direct covalent bond or direct non-covalent interactions) or can be attached via a linker. Exemplary suitable linkers include, without limitation, an optionally substituted alkylene; an optionally substituted alkenylene; an optionally substituted alkynylene; an optionally substituted heteroalkylene; an optionally substituted heteroalkenylene; an optionally substituted heteroalkynylene; an optionally substituted arylene; an optionally substituted heteroarylene; or an optionally substituted acylene, or any combination thereof. The list of suitable linkers is not meant to be limiting, and additional suitable linkers will be apparent to those of skill in the art based on the instant disclosure. It will be appreciated that the label may be attached to or incorporated into a molecule, for example, an IDE-binding molecule at any position. In general, a label can fall into any one (or more) of five classes: a) a label which contains isotopic moieties, which may be radioactive or heavy isotopes, including, but not limited to, ²H, ³H, ¹³C, ¹⁴C, ¹⁵N, ¹⁸F, ³¹P, ³²P, ³⁵S, ⁶⁷Ga, ⁷⁶Br, ^{99m}Tc (Tc-99m), ¹¹¹In, ¹²³I, ¹²⁵I, ¹³¹I, ¹⁵³Gd, ¹⁶⁹Yb, and ¹⁸⁶Re; b) a label which contains an immune moiety, which may be antibodies or antigens, which may be bound to enzymes (e.g., such as horseradish peroxidase); c) a label which is a colored, luminescent, phosphorescent, or fluorescent moieties (e.g., fluorophores such as the fluorescent label fluoresceinisothiocyanat (FITC); d) a label which has one or more photo affinity moieties; and e) a label which is a ligand for one or more known binding partners (e.g., biotin-streptavidin, FK506-FKBP). In certain embodiments, a label comprises a radioactive isotope, preferably an isotope which emits detectable particles, such as β particles. In certain embodiments, the label comprises a fluorescent moiety, also referred to herein as a fluorophore. Suitable fluorophores include, but are not limited to xanthene derivatives, cyanine derivatives, naphthalene derivatives, dansyl or prodan derivatives, coumarin derivatives, oxadiazole derivatives, pyrene derivatives, oxazine derivatives, acridine derivatives, arylmethine derivatives, tetrapyrrole derivatives, quantum dots, fluorescein, rhodamine, Oregon green, eosin, Texas red, cyanine, indocarbocyanine, oxacarbocyanine, thiacarbocyanine, merocyanine, pyridyloxazole, nitrobenzoxadiazole, benzoxadiazole, cascade blue, nile red, nile blue, cresyl violet, oxazine 170, proflavin, acridine orange, acridine yellow, auramine, crystal violet, malachite green, porphin, phthalocyanine, and bilirubin. Suitable fluorophores further include, in some embodiments, fluorescent proteins. In certain embodiments, the label comprises fluoresceinisothiocyanat (FITC). In certain embodiments, the label comprises a ligand moiety with one or more known binding partners. In certain embodiments, the label comprises biotin. In some embodiments, a label is a fluorescent protein (e.g., GFP or a derivative thereof such as enhanced GFP (EGFP)) or a luciferase (e.g., a firefly, Renilla, or Gaussia luciferase). It will be appreciated that, in certain embodiments, a label may react with a suitable substrate (e.g., a luciferin) to generate a detectable signal. Non-limiting examples of fluorescent proteins include GFP and derivatives thereof, proteins comprising chromophores that emit light of different colors such as red, yellow, and cyan fluorescent proteins,. Exemplary fluorescent proteins include, e.g., Sirius, Azurite, EBFP2, TagBFP, mTurquoise, ECFP, Cerulean, TagCFP, mTFP1, mUkG1, mAG1, AcGFP1, TagGFP2, EGFP, mWasabi, EmGFP, TagYPF, EYFP, Topaz, SYFP2, Venus, Citrine, mKO, mKO2, mOrange, mOrange2, TagRFP, TagRFP-T, mStrawberry, mRuby, mCherry, mRaspberry, mKate2, mPlum, mNeptune, T-Sapphire, mAmetrine, mKeima. See, e.g., Chalfie, M. and Kain, S R (eds.) Green fluorescent protein: properties, applications, and protocols (Methods of biochemical analysis, v. 47). Wiley-Interscience, Hoboken, N. J., 2006, and/or Chudakov, D M, et al., Physiol Rev. 90(3):1103-63, 2010 for discussion of GFP and numerous other fluorescent or luminescent proteins. In some embodiments, a label comprises a dark quencher, e.g., a substance that absorbs excitation energy from a fluorophore and dissipates the energy as heat.

The term “effective amount,” as used herein, refers to an amount of a biologically active agent that is sufficient to elicit a desired biological response. For example, in a binding assay in which the ability of a candidate IDE-binding compound to displace an IDE-binding probe is measured, an effective amount of the candidate compound may be an amount sufficient to displace, e.g., release from IDE, at least 105, at least 25%, at least 50%, at least 75%, or 100% of the probe bound to IDE, e.g., as measured by a method described herein. In some embodiments, an effective amount of an IDE inhibitor may refer to an amount of the IDE inhibitor sufficient to reduce an IDE activity by at least 10%, at least 20%, at least 30%, at least 40%, at least 50%, at least 60%, at least 70%, at least 80%, at least 90%, at least 95%, at least 98%, at least 99%, or by 100%, as compared to IDE activity under the same conditions in the absence of the IDE inhibitor. As will be appreciated by the skilled artisan, the effective amount of an agent, e.g., an IDE inhibitor, may vary depending on various factors as, for example, on the desired biological response, the specific conditions (e.g., in vitro conditions or in vivo conditions), the cell or tissue being targeted, and the specific agent being used. The term “therapeutically effective amount,” as used herein, refers to the amount or concentration of an inventive compound, that, when administered to a subject, is effective to at least partially treat a condition from which the subject is suffering. In some embodiments, an effective amount of an IDE inhibitor is an amount the administration of which results in inhibition of at least about 50%, at least about 60%, at least about 70%, at least about 75%, at least about 80%, at least about 90%, at least about 95%, at least about 98%, at least about 99%, at least about 99.5%, or about 100% of IDE activity as compared to a baseline level, for example, a level of IDE activity in the absence of the inhibitor.

As used herein the term “inhibit” or “inhibition” in the context of enzymes, for example, in the context of IDE, refers to a reduction in the activity of the enzyme. In some embodiments, the term refers to a reduction of the level of enzyme activity, e.g., IDE activity, to a level that is statistically significantly lower than an initial level, which may, for example, be a baseline level of enzyme activity. In some embodiments, the term refers to a reduction of the level of enzyme activity, e.g., IDE activity, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of enzyme activity.

As used herein, the term “insulin degrading enzyme” or “IDE” refers to an insulin-degrading enzyme. IDE (also referred to herein as IDE proteins) and their respective encoding RNA and DNA sequences according to some aspects of this invention include human IDE protein and encoding sequences, as well as, in some embodiments, IDE proteins and encoding sequences from other species, for example, from other mammals (e.g., IDE proteins and encoding sequences from mouse, rat, cat, dog, cattle, goat, sheep, pig, or primate), from other vertebrates, and from insects. In some embodiments, an IDE inhibitor provided herein is specific for an IDE from a species, e.g., for human IDE, mouse IDE, rat IDE, and so on. In some embodiment, an IDE provided herein inhibits IDEs from more than one species, e.g., human IDE and mouse IDE. In some embodiments, an IDE provided herein exhibits equipotent inhibition of IDEs from more than one species, e.g., equipotent inhibition of human and mouse IDEs. The term IDE further includes, in some embodiments, sequence variants and mutations (e.g., naturally occurring or synthetic IDE sequence variants or mutations), and different IDE isoforms. In some embodiments, the term IDE includes protein or encoding sequences that are homologous to an IDE protein or encoding sequence, for example, a protein or encoding sequence having at least 80%, at least 85%, at least 90%, at least 91%, at least 92%, at least 93%, at least 94%, at least 95%, at least 96%, at least 97%, at least 98%, at least 99%, or at least 99.5% sequence identity with an IDE sequence, for example, with an IDE sequence provided herein. In some embodiments, the term IDE refers to a protein exhibiting IDE activity, for example, a protein exhibiting insulin-targeted protease activity, or a nucleic acid sequence encoding such a protein. In some embodiments, the term IDE included proteins that exhibit at least 50%, at least 60%, at least 70%, at least 80%, at least 90%, at least 95%, or at least 100% insulin-targeting protease activity as compared to a known IDE protein or encoding sequence, for example, as compared to an IDE sequence provided herein. IDE protein and encoding gene sequences are well known to those of skill in the art, and exemplary protein sequences include, but are not limited to, the following sequences. Additional IDE sequences, e.g., IDE homologues from other mammalian species, will be apparent to those of skill in the art, and the invention is not limited to the exemplary sequences provided herein.

>gi|155969707|ref|NP_004960.2| insulin-degrading
enzyme isoform 1 [Homo sapiens]
(SEQ ID NO: 1)
MRYRLAWLLHPALPSTFRSVLGARLPPPERLCGFQKKTYSKMNNPAIKRI
GNHITKSPEDKREYRGLELANGIKVLLISDPTTDKSSAALDVHIGSLSDP
PNIAGLSHFCEHMLFLGTKKYPKENEYSQFLSEHAGSSNAFTSGEHTNYY
FDVSHEHLEGALDRFAQFFLCPLFDESCKDREVNAVDSEHEKNVMNDAWR
LFQLEKATGNPKHPFSKFGTGNKYTLETRPNQEGIDVRQELLKFHSAYYS
SNLMAVCVLGRESLDDLTNLVVKLFSEVENKNVPLPEFPEHPFQEEHLKQ
LYKIVPIKDIRNLYVTFPIPDLQKYYKSNPGHYLGHLIGHEGPGSLLSEL
KSKGWVNTLVGGQKEGARGFMFFIINVDLTEEGLLHVEDIILHMFQYIQK
LRAEGPQEWVFQECKDLNAVAFRFKDKERPRGYTSKIAGILHYYPLEEVL
TAEYLLEEFRPDLIEMVLDKLRPENVRVAIVSKSFEGKTDRTEEWYGTQY
KQEAIPDEVIKKWQNADLNGKFKLPTKNEFIPTNFEILPLEKEATPYPAL
IKDTAMSKLWFKQDDKFFLPKACLNFEFFSPFAYVDPLHCNMAYLYLELL
KDSLNEYAYAAELAGLSYDLQNTIYGMYLSVKGYNDKQPILLKKIIEKMA
TFEIDEKRFEIIKEAYMRSLNNFRAEQPHQHAMYYLRLLMTEVAWTKDEL
KEALDDVTLPRLKAFIPQLLSRLHIEALLHGNITKQAALGIMQMVEDTLI
EHAHTKPLLPSQLVRYREVQLPDRGWFVYQQRNEVHNNCGIEIYYQTDMQ
STSENMFLELFCQIISEPCFNTLRTKEQLGYIVFSGPRRANGIQGLRFII
QSEKPPHYLESRVEAFLITMEKSIEDMTEEAFQKHIQALAIRRLDKPKKL
SAECAKYWGEIISQQYNFDRDNTEVAYLKTLTKEDIIKFYKEMLAVDAPR
RHKVSVHVLAREMDSCPVVGEFPCQNDINLSQAPALPQPEVIQNMTEFKR
GLPLFPLVKPHINFMAAKL
>gi|260099676|ref|NP_001159418.1| insulin-
degrading enzyme isoform 2 [Homo sapiens]
(SEQ ID NO: 2)
MSKLWFKQDDKFFLPKACLNFEFFSPFAYVDPLHCNMAYLYLELLKDSLN
EYAYAAELAGLSYDLQNTIYGMYLSVKGYNDKQPILLKKIIEKMATFEID
EKRFEIIKEAYMRSLNNFRAEQPHQHAMYYLRLLMTEVAWTKDELKEALD
DVTLPRLKAFIPQLLSRLHIEALLHGNITKQAALGIMQMVEDTLIEHAHT
KPLLPSQLVRYREVQLPDRGWFVYQQRNEVHNNCGIEIYYQTDMQSTSEN
MFLELFCQIISEPCFNTLRTKEQLGYIVFSGPRRANGIQGLRFIIQSEKP
PHYLESRVEAFLITMEKSIEDMTEEAFQKHIQALAIRRLDKPKKLSAECA
KYWGEIISQQYNFDRDNTEVAYLKTLTKEDIIKFYKEMLAVDAPRRHKVS
VHVLAREMDSCPVVGEFPCQNDINLSQAPALPQPEVIQNMTEFKRGLPLF
PLVKPHINFMAAKL
<gi|121583922|ref|NP_112419.2| insulin-
degrading enzyme [Mus musculus]
(SEQ ID NO: 3)
MRNGLVWLLHPALPGTLRSILGARPPPAKRLCGFPKQTYSTMSNPAIQRI
EDQIVKSPEDKREYRGLELANGIKVLLISDPTTDKSSAALDVHIGSLSDP
PNIPGLSHFCEHMLFLGTKKYPKENEYSQFLSEHAGSSNAFTSGEHTNYY
FDVSHEHLEGALDRFAQFFLCPLFDASCKDREVNAVDSEHEKNVMNDAWR
LFQLEKATGNPKHPFSKFGTGNKYTLETRPNQEGIDVREELLKFHSTYYS
SNLMAICVLGRESLDDLTNLVVKLFSEVENKNVPLPEFPEHPFQEEHLRQ
LYKIVPIKDIRNLYVTFPIPDLQQYYKSNPGHYLGHLIGHEGPGSLLSEL
KSKGWVNTLVGGQKEGARGFMFFIINVDLTEEGLLHVEDIILHMFQYIQK
LRAEGPQEWVFQECKDLNAVAFRFKDKERPRGYTSKIAGKLHYYPLNGVL
TAEYLLEEFRPDLIDMVLDKLRPENVRVAIVSKSFEGKTDRTEQWYGTQY
KQEAIPEDIIQKWQNADLNGKFKLPTKNEFIPTNFEILSLEKDATPYPAL
IKDTAMSKLWFKQDDKFFLPKACLNFEFFSPFAYVDPLHCNMAYLYLELL
KDSLNEYAYAAELAGLSYDLQNTIYGMYLSVKGYNDKQPILLKKITEKMA
TFEIDKKRFEIIKEAYMRSLNNFRAEQPHQHAMYYLRLLMTEVAWTKDEL
KEALDDVTLPRLKAFIPQLLSRLHIEALLHGNITKQAALGVMQMVEDTLI
EHAHTKPLLPSQLVRYREVQLPDRGWFVYQQRNEVHNNCGIEIYYQTDMQ
STSENMFLELFCQIISEPCFNTLRTKEQLGYIVFSGPRRANGIQGLRFII
QSEKPPHYLESRVEAFLITMEKAIEDMTEEAFQKHIQALAIRRLDKPKKL
SAECAKYWGEIISQQYNYDRDNIEVAYLKTLTKDDIIRFYQEMLAVDAPR
RHKVSVHVLAREMDSCPVVGEFPSQNDINLSEAPPLPQPEVIHNMTEFKR
GLPLFPLVKPHINFMAAKL

As used herein, the terms “treatment,” “treat,” and “treating” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. In some embodiments, the disease or disorder being treated is associated with aberrant IDE activity, or can be treated by inhibiting IDE activity. In some embodiments, the disease is metabolic syndrome, pre-diabetes, or diabetes. In some embodiments, the disease is diabetes or metabolic syndrome in a subject with Alzheimer's Disease or at risk of developing Alzheimer's Disease.

### DETAILED DESCRIPTION OF CERTAIN EMBODIMENTS OF THE INVENTION

The discovery and application of the first potent, highly selective, and physiologically active small-molecule IDE inhibitor revealed that IDE inhibition can lead to improved glucose tolerance in lean and obese mice after oral glucose administration. It is thus desirable to identify additional IDE inhibitors such as other chemical classes of IDE inhibitors that can be developed for human clinical use or for research purposes, and to develop clinical administration regimens for IDE inhibition in the treatment and/or prevention of disease (e.g., diabetes).

Some aspects of this disclosure provide methods, assays, and reagents that are useful for identifying physiologically active IDE inhibitors, and, in some embodiments, for identifying compounds that can bind IDE with a desired affinity or at a specific binding site. The binding site may not be the active site of IDE.

Some aspects of this disclosure provide physiologically active small-molecule probes that bind IDE with high affinity. In some embodiments, the probes disclosed herein are useful for performing competitive binding assays, for example, fluorescence polarization assays, to identify compounds that bind IDE.

Some aspects of this disclosure provide methods of administering an IDE inhibitor to a subject, either alone or in combination with another therapeutic, in order to improve insulin signaling. In some embodiments, these methods are useful for improving oral glucose tolerance, to modulate gastric emptying, and/or to modulate satiety during, after, and/or in the time period between meals in a subject, for example, in a subject having diabetes.

### Methods for Identifying IDE-Binding Compounds

Some aspects of this disclosure provide methods that are useful for identifying molecules that bind IDE and/or determining the IDE-binding properties of a candidate compound. In some embodiments, the methods provided herein are useful to determine competitive IDE-binding of a candidate compound in the presence of an IDE-binding probe. In general, such methods include contacting an IDE with a candidate compound in the presence of the IDE-binding probe, and identifying a candidate compound as an IDE-binding compound, if the candidate compound is able to bind IDE, thus replacing some or all of the bound probe. The use of an IDE-binding probe as a competitor for IDE-binding is advantageous over non-competitive binding assays because it circumvents the requirement to directly assess the interaction of a candidate compound with IDE or the modulation of IDE activity by a candidate compound, thus allowing for screening a variety of candidate compound structures with a single type of assay, or using a single probe. The use of competitive binding assays also allows for decreasing the incidence of low-affinity binders by using a high-affinity probe, and for identifying candidates that compete with the probe for a specific IDE binding site. Some aspects of this disclosure provide IDE-binding probes designed to bind a unique site of IDE, which contributes to the probes high specificity for binding IDE.

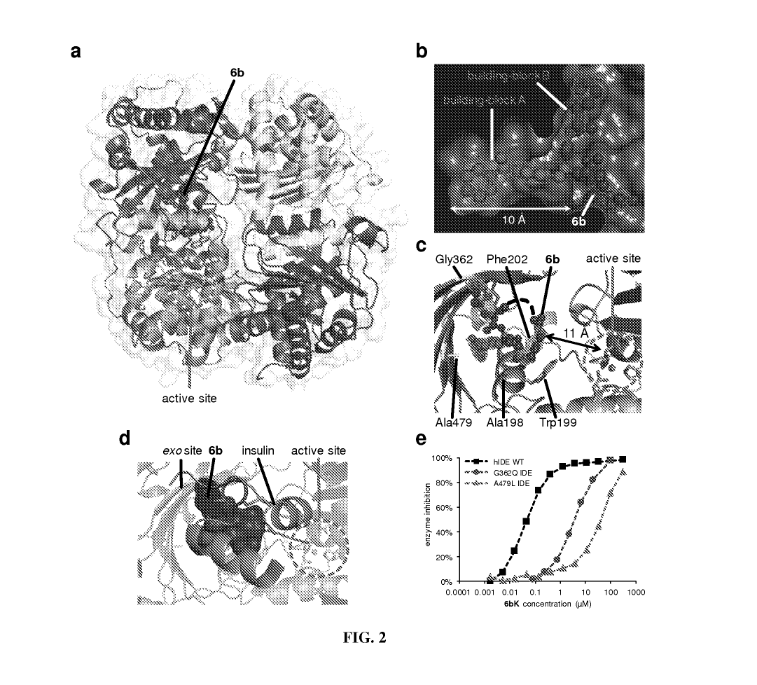

Some aspects of this disclosure relate to structural insights regarding IDE binding sites described herein. Briefly, it was found that a potent IDE inhibitor, macrocycle 6b, occupies a binding pocket at the interface of IDE domains 1 and 2 and is positioned more than 11 Å away from the zinc ion in the IDE active site. This site does not overlap with the binding site of the substrate-mimetic inhibitor Ii1, suggesting that the macrocycle competes with substrate binding and abrogates key interactions that are necessary to bind peptide substrates for cleavage (see FIG. 2). Accordingly, where it is desirable to identify a compound binding a specific IDE site, e.g., the binding pocket for macrocycle 6b, the use of a compound binding the IDE site as a competitive probe in a binding assay will minimize the false-positive identification of candidate compounds that bind non-overlapping sites. The assays, methods, systems, kits, and reagents provided herein are thus useful in the identification of candidates that specifically target such binding sites, and in the discovery of novel IDE-inhibitor therapeutics and leads thereto.

In general, the methods for identifying insulin-degrading enzyme (IDE)-binding compounds described herein comprise contacting an IDE with a probe that binds IDE. The probe typically comprises a detectable label, which allows for or facilitates the detection of the probe in its bound and/or unbound state. The method further comprises contacting the IDE with a candidate compound. The contacting is performed under conditions suitable and for a time sufficient for the probe and the candidate compound to bind the IDE. The method further comprises determining the level of unbound probe in the presence of the candidate compound, for example, via one of the detection methodologies described herein or a suitable methodology known in the art. The method further comprises comparing the level of unbound probe to a reference level, and identifying the candidate compound as an IDE-binding compound, if the level of unbound probe in the presence of the candidate compound is higher than the reference level.

In some embodiments, the IDE-binding probe comprises a macrocyclic IDE-binding molecule, e.g., a macrocyclic molecule as described herein, or a macrocyclic molecule as described in international PCT application PCT/US2012/044977, filed Jun. 29, 2012, entitled “Macrocyclic Insulin-Degrading Enzyme (IDE) Inhibitors and Uses Thereof,” as published under Publication No. WO/2013/006451 on Jan. 10, 2013, the entire contents of which are incorporated herein by reference. In some embodiments, the probe binds IDE with an IC₅₀ of 50 μM or less, e.g., with an IC₅₀ of 10 μM or less, of 5 μM or less, of 4 μM or less, of 3 μM or less, of 2.5 μM or less, of 2 μM or less, of 1 μM or less, of 500 nM or less, of 400 nM or less, of 300 nM or less, of 250 nM or less, of 200 nM or less, of 100 nM or less, of 50 nM or less, of 40 nM or less, of 30 nM or less, of 25 nM or less, of 10 nM or less, of 5 nM or less, of 2.5 nM or less, or of 1 nM or less.

In some embodiments, the macrocyclic IDE-binding molecule comprises a compound of any of Formula (I)-(VI). In some embodiments, the macrocyclic IDE-binding molecule comprises a structure selected from the group consisting of structures 6b, 2b, 3b, 4b, 5b, 6a, 6c, 6b, 6bk, and 7-29. In some embodiments, the macrocyclic IDE-binding molecule comprises structure 6bk.

In some embodiments, the macrocyclic IDE-binding molecule is conjugated to the detectable label. In some embodiments, the conjugation is via a direct, covalent bond of the detectable label to the IDE-binding molecule. In some embodiments, the conjugation is via a linker. In some embodiments, the detectable label is conjugated to the IDE-binding molecule in a manner that does not interfere with the IDE-binding properties of the molecule.

In some embodiments, the detectable label comprises a fluorophore. In some embodiments, the detectable label comprises a non-protein fluorophore, such as, for example, a xanthene derivative, a cyanine derivative, a naphthalene derivative, a dansyl or prodan derivative, a coumarin derivative, an oxadiazole derivative, a pyrene derivative, an oxazine derivative, an acridine derivative, an arylmethine derivative, or a tetrapyrrole derivative. In some embodiments, the detectable label comprises fluorescein, rhodamine, Oregon green, eosin, Texas red, cyanine, indocarbocyanine, oxacarbocyanine, thiacarbocyanine, merocyanine, pyridyloxazole, nitrobenzoxadiazole, benzoxadiazole, cascade blue, nile red, nile blue, cresyl violet, oxazine 170, proflavin, acridine orange, acridine yellow, auramine, crystal violet, malachite green, porphin, phthalocyanine, or bilirubin. In some embodiments, the detectable label comprises a fluorescent protein. In some embodiments, the detectable label comprises a quantum dot. In some embodiments, the probe comprises compound 31.

In some embodiments, the method comprises a fluorescence polarization assay. Fluorescence polarization assay technology is well known to those of skill in the art. See, e.g., Lea et al., “Fluorescence Polarization Assays in Small Molecule Screening,” Expert Opin Drug Discov. 2011 January; 6(1): 17-32, the entire contents of which are incorporated herein by reference. The fluorescence polarization assays described herein provide a di0rect, nearly instantaneous measure of an IDE-binding probe's bound/free ratio. The fluorescence polarization assays provided herein are based on the properties of fluorescent molecules in solution, which, when excited with plane-polarized light, will emit polarized light into the same plane as the incident light if the molecules remain stationary during the excitation of the fluorophore. If the fluorescent molecules rotate or tumble during excitation, however, the emitted fluorescent light will be emitted into a plane different from the incident light, thus decreasing the level of polarization of the emitted light. The emitted fluorescent light can be measured with suitable detectors and the level of polarization of the emitted light can be determined. In some embodiments, the level of polarization is calculated, e.g., as the fluorescent anisotropy.

Suitable methods for measuring fluorescent polarization, suitable detectors, fluorophores, incident light parameters, reaction conditions, exposure times, and algorithms for calculating the degree of fluorescent polarization are known to those of skill in the art and include, but are not limited to, those described in Perrin, Polarization of light of fluorescence, average life of molecules. J Phys Radium. 1926; 7:390-401; Owicki J C. Fluorescence polarization and anisotropy in high throughput screening: perspectives and primer. J Biomol Screen. 2000; 5(5):297-306; Burke T J, Loniello K R, Beebe J A, et al. Development and application of fluorescence polarization assays in drug discovery. Comb Chem High Throughput Screen. 2003; 6(3):183-94; Jameson D M, Croney J C. Fluorescence polarization: past, present and future. Comb Chem High Throughput Screen. 2003; 6(3):167-73; Nasir M S, Jolley M E. Fluorescence polarization: an analytical tool for immunoassay and drug discovery. Comb Chem High Throughput Screen. 1999; 2(4):177-90; Jameson D M, Ross J A. Fluorescence polarization/anisotropy in diagnostics and imaging. Chem Rev. 2010; 110(5):2685-708; and Gradinaru C C, Marushchak D O, Samim M, et al. Fluorescence anisotropy: from single molecules to live cells. Analyst. 2010; 135(3):452-9; the entire contents of each of which are incorporated herein by reference.

In some embodiments, a fluorescence polarization assay is used to determine whether a candidate compound binds to IDE in the presence of an IDE-binding fluorescent probe. Without wishing to be bound by any particular theory, it is believed that the polarization of light emitted by a molecule is proportional to the molecule's rotational relaxation time, which varies with molecular volume, amongst other parameters. A small molecule, e.g., a free IDE-binding fluorescent probe as described herein, will thus be able to rotate and tumble more freely than an IDE-binding fluorescent probe that is bound to the much larger IDE molecule. Accordingly, in some embodiments, the determination of whether a candidate compound binds IDE is based on the theory that the rotation and tumbling of the probe is hindered when the probe is bound to IDE, while the probe can rotate and tumble freely once it is replaced from its IDE-binding site by the candidate compound. Accordingly, the level of polarization of light emitted from a fluorescent IDE-binding probe bound to an IDE will be higher in the absence of a candidate that is capable of successfully competing with the probe for the binding pocket, thus releasing the bound probe into the surrounding media where it can rotate and tumble freely, as compared to the level of polarization in the presence of a candidate compound that binds IDE with high affinity and replaces the probe at the IDE-binding site.

In some embodiments, vertically polarized light is used to excite the fluorophore of the probe, and the level of polarization of the emitted light is monitored in vertical and horizontal planes, e.g., by measuring the emission intensity in both planes and determining the degree of movement of emission from the vertical to the horizontal plane. As the degree of movement or the degree of polarization is related to the mobility of the fluorescent probe, and the mobility of the probe increases when it is replaced from its IDE binding site by a candidate compound, the measures shift in the level of polarization can be used to calculate the degree of probe replacement or candidate compound binding.

In some embodiments, the fluorescence polarization assay is performed repeatedly on an IDE contacted with a probe and a candidate compound. In some embodiments, the assay conditions are changed between repetitions, e.g., the pH, salt concentration, or temperature is adjusted, the concentration of the IDE, the probe, and/or the candidate compound is altered, or another compound binding IDE, e.g., an IDE substrate, is added. In some embodiments, the fluorescence polarization measurement is performed after a time sufficient for the binding reaction reaching equilibrium. In some embodiments, the fluorescence polarization measurement is performed at a plurality of time points (including in real-time) before the binding reaction reaches equilibrium. In some such embodiments, the time in which the binding reaction (e.g., replacement of the bound probe by the candidate compound) reaches equilibrium and/or the kinetics of the binding reaction are measured.

The fluorescence polarization assays described herein are advantageous over some other methods for studying the binding of candidate compounds to IDE, for example, in that they typically have a low limit of detection (typically in the sub-nanomolar range), are truly homogeneous, thus allowing the observation of binding reactions in the absence of solid supports or other agents or structures required for separation of bound and unbound probe.

In some embodiments, the florescence polarization method comprises contacting an IDE with a probe comprising a fluorophore and with a candidate compound under conditions and for a time sufficient for the probe and the candidate compound to bind IDE, exposing the IDE contacted with the probe and the compound to polarized light of a suitable wave length to excite the fluorophore, and measuring the degree of polarization of the light emitted from the fluorophore. In some embodiments, the method comprises calculating the degree of polarization of the emitted light as the fluorescence anisotropy. In some embodiments, the degree of fluorescent polarization (e.g., calculated as fluorescent anisotropy) is proportional to the degree of binding of the candidate molecule to the IDE molecule.

In some embodiments, the method comprises identifying a candidate molecule as an IDE-binding molecule when the degree of polarization is decreased by at least 5%, at least 10%, at least 15%, at least 20%, at least 25%, at least 30%, at least 35%, at least 40%, at least 45%, at least 50%, at least 55%, at least 60%, at least 70%, at least 75%, at least 80%, at least 85%, at least 90%, at least 95%, at least 98%, at least 99%, or by 100%.

In some embodiments, the method comprises determining the level of unbound probe in the presence of the candidate compound. In some embodiments, the method comprises exposing the IDE molecule contacted with the probe and the candidate compound to incident, plane-polarized light of a suitable wave length to excite the fluorophore; and detecting the level of fluorescent light emitted by the fluorophore in the same plane of polarization as the incident light, as well as the level of fluorescent light emitted by the fluorophore in a plane different from the plane of polarization of the incident light. In some embodiments, determining the level of unbound probe in the presence of the candidate compound comprises calculating the level of unbound probe from the levels of emitted light detected. In some embodiments, the calculating the level of unbound probe comprises calculating a ratio of the levels of emitted light detected, or calculating a fluorescence anisotropy value.

In some embodiments, the level of polarization is determined from measurements of emitted fluorescence intensities in different planes, e.g., in a plane parallel and a plane perpendicular to the plane of polarized excitation light.

In some embodiments, the level of polarization is calculated as fluorescence polarization (P):

$$P = \frac{( {F}_{\vert \vert} - {F}_{⊥} )}{( {F}_{\vert \vert} + {F}_{⊥} )} ,$$

wherein

F_{∥}=fluorescence intensity parallel to excitation plane, and

F^{⊥}=fluorescence intensity perpendicular to excitation plane.

In some embodiments, the level of polarization is calculated as fluorescence anisotropy (r):

$$r = \frac{( {F}_{\vert \vert} - {F}_{⊥} )}{( {F}_{\vert} + 2 {F}_{⊥} )} ,$$

wherein

F_{∥}=fluorescence intensity parallel to excitation plane, and

F^{⊥}=fluorescence intensity perpendicular to excitation plane.

In some embodiments, the candidate compound is identified as an IDE-binding compound if the level of fluorescent light emitted by the fluorophore in the presence of the candidate compound in a plane different from the plane of polarization of the incident light is higher than a reference level of fluorescent light emitted in that plane measured in the absence of the candidate compound. In some embodiments, the method is carried out repeatedly for a candidate compound at a plurality of IDE concentrations, and the method comprises calculating a ratio of the levels of emitted light detected, or calculating a fluorescence anisotropy value for each concentration; and determining a dynamic IDE concentration range.

In some embodiments, the candidate compound is identified as an IDE-binding compound if the level of fluorescent light emitted by the fluorescent probe in the presence of the candidate compound in a plane different from the plane of polarization of the incident light is at least 1.1-fold, at least 1.2-fold, at least 1.3-fold, at least 1.5-fold, at least 1.75-fold, at least 2-fold, at least 2.5-fold, at least 3-fold, at least 4-fold, at least 5-fold, at least 10-fold, at least 20-fold, at least 30-fold, at least 40-fold, at least 50-fold, at least 100-fold, at least 200-fold, at least 500-fold, or at least 1000-fold higher than a reference level of fluorescent light emitted in that plane measured in the absence of the candidate compound.

In some embodiments, the candidate compound is identified as an IDE-binding compound if the fluorescence anisotropy in the presence of the candidate compound is at least 1.1-fold, at least 1.2-fold, at least 1.3-fold, at least 1.5-fold, at least 1.75-fold, at least 2-fold, at least 2.5-fold, at least 3-fold, at least 4-fold, at least 5-fold, at least 10-fold, at least 20-fold, at least 30-fold, at least 40-fold, at least 50-fold, at least 100-fold, at least 200-fold, at least 500-fold, or at least 1000-fold lower than the fluorescence anisotropy in the absence of the candidate compound. In some embodiments, the level of emitted light or the fluorescent anisotropy is measured at a point within the dynamic IDE concentration range.

In some embodiments, the screening methods provided herein identify compounds that modulate (e.g., inhibit or activate) IDE based on their interaction with a binding site pocket defined by Leu201, Gly205, Tyr302, Thr316, and Ala479 of IDE, and in some embodiments, nearby residues. In some embodiments, a probe is used that binds this pocket with high specificity. In some embodiments, such methods for identifying highly-IDE selective compounds that bind to this site include the use of a probe described herein (e.g., 6b, 6bk, 31, etc.) in a competitive binding assay, as described herein, e.g., a fluorescence polarization-based, fluorescence resonance energy transfer (FRET)-based, other fluorescence-based, antibody-based, solid-support-based, or anisotropy-based assay.

Additional suitable assays and detection technologies that can be used to determine the level of unbound probe in the presence of a candidate compound, e.g., a candidate IDE-modulating compound, in order to identify IDE-binding and/or IDE-modulating compounds. Such suitable assays and detection technologies include, without limitation, fluorescence-based, antibody-based, anisotropy-based, plasmon resonance-based, and fluorescence resonance energy transfer (FRET)-based assays and readouts. Additional suitable assays and detection technologies will be apparent to those of skill in the art based on the instant disclosure, and the disclosure is not limited in this respect.

In some embodiments, the detectable label comprises a binding agent. In some embodiments, the binding agent comprises an antibody or an antigen-binding fragment thereof. In some embodiments, the binding agent comprises a ligand. In some embodiments, the ligand is biotin or an avidin derivative. In some embodiments, the probe comprises compound 30. In some embodiments, the detectable label comprises a detectable isotope. In some embodiments, the detectable isotope is selected from the group consisting of ²H, ³H, ¹³C, ¹⁴C, ¹⁵N, ¹⁸F, ³¹P, ³²P, ³⁵S, ⁶⁷Ga, ⁷⁶Br, ^{99m}Tc (Tc-99m), ¹¹¹In, ¹²³I, ¹²⁵I, ¹³¹I, ¹⁵³Gd, ¹⁶⁹Yb, and ¹⁸⁶Re. In some such embodiments, bound probe and free probe are physically separated and then quantified to determine the level of probe released from IDE by a candidate compound. This can be achieved, for example, by exposing the IDE contacted with the probe and the candidate compound to conditions resulting in the precipitation of the IDE proteins in solution, together with any IDE-bound probe, but allow any unbound probe to remain in solution. Suitable precipitation conditions will be apparent to those of skill in the art and include, but are not limited to those conditions described in Dennison, A Guide to Protein Isolation, Publisher: Springer; 2nd edition (Apr. 30, 2003), ISBN-10: 1402012241; the entire contents of which are incorporated herein by reference. In other embodiments, the probe and any bound IDE protein is separated based on the binding agent comprised in the probe, e.g., via attachment of a probe comprising biotin to a solid support comprising an avidin derivative, such as streptavidin. The total amount of bound IDE protein retrieved in this manner can then be assessed by standard methods, and compared to the amount of bound IDE protein retrieved in the absence of a candidate compound.

In some embodiments, the IDE is contacted with the probe and the candidate compound in aqueous solution. In some embodiments, the IDE is contacted with the probe and the candidate compound under physiological conditions.

In some embodiments, the method comprises screening a library of different candidate compounds. In some embodiments, the library comprises at least 10¹, at least 10², at least 10³, at least 10⁴, at least 10⁵, or at least 10⁶ different candidate compounds. In some embodiments, the method comprises a parallel assessment of a plurality of different candidate compounds, for example, in a multi-well plate format.

In some embodiments, a suitable reference level to which a measurement in the presence of a candidate compound is compared represents a measurement made under the same circumstances but in the absence of a candidate compound. In some embodiments, the reference level is a level of fluorescent polarization, an intensity of light emitted in a plane different from the incident light used, or a fluorescence anisotropy determined in the absence of a candidate compound. In some embodiments, the reference level is determined by measuring the level of unbound probe in the presence of a control compound with known IDE-binding properties. For example, in some embodiments, the reference level is determined by measuring the level of unbound probe in the presence of an IDE-binding compound that binds IDE with an IC₅₀ of more than 10 μM, more than 100 μM, more than 1 mM, or more than 10 mM.

In some embodiments, the probe comprises an IDE-inhibitor, for example, an IDE inhibitor of formula (I)-(VIII), 1b, 2b, 3b, 4b, 5b, 6a, 6b, 6bK, 6c,or 1-31. In some such embodiments, if a candidate compound is identified as an IDE-binding compound, then the compound is also identified as an IDE-inhibiting compound.

### Methods for Detecting IDE

The probes and detection methods described herein can also be used to determine an amount of IDE or a level of IDE activity in a sample, e.g., in a biological sample obtained from a subject. In some embodiments, the biological sample comprises a cell, a tissue, and/or a body fluid obtained from the subject. Exemplary body fluids include, without limitation, blood, serum, plasma, saliva, and urine. In some embodiments, a method for determining an amount of IDE or a level of IDE activity as provided herein comprises contacting a biological sample obtained from a subject with an IDE-binding probe described herein, and determining the amount or level of probe binding to IDE, if any, e.g., by a suitable detection assay provided herein.

For example, in some embodiments, such a method may comprise contacting a biological sample obtained from a subject with a probe that selectively binds IDE, e.g., a probe that comprises a macrocyclic compound conjugated to a detectable label, such as, for example, a compound comprising a structure selected from the group consisting of structures 1b, 2b, 3b, 4b, 5b, 6a, 6c, 6b, 6bk, and 1-31. In some embodiments the amount of probe binding to IDE in the sample is quantified, for example, by an assay described herein, or by any other suitable assay. In some embodiments, the amount of probe binding to IDE in the biological sample is quantified by a fluorescence polarization assay, a fluorescence resonance energy transfer (FRET) assay, a fluorescence-based assay, an antibody-based assay, a solid-support-based assay, or an anisotropy assay. In some embodiments, the determined amount of IDE-bound probe is compared to a reference level, e.g., to a level of probe bound to IDE in a sample from a healthy subject, or an average level of probe bound to IDE in a population of subjects (e.g., age- and/or gender-matched subjects), or to a level representative of a healthy subject.

In some embodiments, if the amount of bound probe in the biological sample is higher, e.g., statistically significantly higher, than the reference level, the subject is indicated to exhibit an overabundance of IDE. In some embodiments, if the amount of bound probe in the biological sample is lower, e.g., statistically significantly lower, than the reference level, the subject is indicated to exhibit a deficiency of IDE. In some embodiments, the subject is diagnosed to have or to be predisposed to develop a disease associated with an aberrant level of IDE based on the level of IDE in the subject being higher or lower than the reference level. For example, in some such embodiments, the subject is diagnosed to have or to be predisposed to develop diabetes, pre-diabetes, Alzheimer's disease, metabolic syndrome, neurodegeneration, mental disorders, and/or cancer. In some embodiments, the subject is selected for a regimen of appropriate health care to treat or prevent the respective disorder.

### Labeled IDE Inhibitor Probes

Some aspects of this disclosure provide IDE-binding compounds that are conjugated to a detectable label. Such compounds are useful as probes in the methods for identifying IDE-binding compounds described herein. Some aspects of this disclosure provide probes comprising a macrocyclic IDE inhibitor conjugated to a detectable label.

In some embodiments, this disclosure provides a labeled IDE inhibitor of the Formula (I):

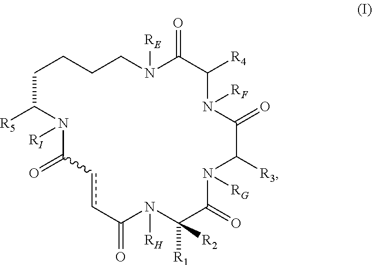

or pharmaceutically acceptable salts, solvates, hydrates, stereoisomers, polymorphs, tautomers, and isotopically enriched forms thereof,
wherein:

[symbol] is a single or double C—C bond, wherein when [symbol] is a double C—C bond, then [symbol] indicates that the adjacent C—C double bond is in a cis or trans configuration;

R₁ is hydrogen; halogen; substituted or unsubstituted aliphatic; substituted or unsubstituted heteroaliphatic; substituted or unsubstituted aryl; substituted or unsubstituted heteroaryl; substituted or unsubstituted acyl; —OR_{A}; —N(R_{A})₂; —SA_{A}; ═O; —CN; —NO₂; —SCN; —SOR_{A}; or —SO₂R_{A}; wherein each occurrence of R_{A} is independently hydrogen; a protecting group; substituted or unsubstituted aliphatic; substituted or unsubstituted heteroaliphatic; substituted or unsubstituted acyl; substituted or unsubstituted aryl; or substituted or unsubstituted heteroaryl;

R₂ is hydrogen; halogen; substituted or unsubstituted aliphatic; substituted or unsubstituted heteroaliphatic; substituted or unsubstituted aryl; substituted or unsubstituted heteroaryl; substituted or unsubstituted acyl; —OR_{B}; —N(R_{B})₂; —SR_{B}; ═O; —CN; —NO₂; —SCN; —SOR_{B}; or —SO₂R_{B}; wherein each occurrence of R_{B} independently hydrogen; a protecting group; substituted or unsubstituted aliphatic; substituted or unsubstituted heteroaliphatic; substituted or unsubstituted acyl; substituted or unsubstituted aryl; or substituted or unsubstituted heteroaryl;

R₃ is hydrogen; halogen; substituted or unsubstituted aliphatic; substituted or unsubstituted heteroaliphatic; substituted or unsubstituted aryl; substituted or unsubstituted heteroaryl; substituted or unsubstituted acyl; —OR_{C}; —N(R_{C})_{y}; —SR_{C}; ═O; —CN; —NO₂; —SCN; —SOR_{C}; or —SO₂R_{C}; wherein y is 0, or an integer between 1-2, inclusive, and wherein each occurrence of R_{C} is independently hydrogen; a protecting group; substituted or unsubstituted aliphatic; substituted or unsubstituted heteroaliphatic; substituted or unsubstituted acyl; substituted or unsubstituted aryl; or substituted or unsubstituted heteroaryl;

R₄ is hydrogen; halogen; substituted or unsubstituted aliphatic; substituted or unsubstituted heteroaliphatic; substituted or unsubstituted aryl; substituted or unsubstituted heteroaryl; substituted or unsubstituted acyl; —OR_{C}; —N(R_{D})_{y}; —SR_{D}; ═O; —CN; —NO₂; —SCN; —SOR_{D}; or —SO₂R_{D}; wherein y is 0, or an integer between 1-2, inclusive, and wherein each occurrence of R_{D} is independently hydrogen; a protecting group; substituted or unsubstituted aliphatic; substituted or unsubstituted heteroaliphatic; substituted or unsubstituted acyl; substituted or unsubstituted aryl; or substituted or unsubstituted heteroaryl

R₅ comprises a detectable label and, optionally, a linker; and

each instance of R_{E}, R_{F}, R_{G}, R_{H}, and R_{I} is independently hydrogen; substituted or unsubstituted acyl; a nitrogen protecting group; substituted or unsubstituted aliphatic; substituted or unsubstituted heteroaliphatic; substituted or unsubstituted aryl; substituted or unsubstituted heteroaryl; substitute or unsubstituted hydroxyl; substituted or unsubstituted thiol; substituted or unsubstituted amino; or halogen; optionally wherein an R₄ group and R_{F} are joined to form a substituted or unsubstituted heterocyclic ring; an R₃ group and R_{G} are joined to form a substituted or unsubstituted heterocyclic ring; and/or an R₁ or R₂ group and R_{II} are joined to form a substituted or unsubstituted heterocyclic ring. In some embodiments, R_{E}, R_{F}, R_{G}, R_{H}, and R_{I} are all H.

In some embodiments, the labeled IDE inhibitors are of Formula (II):

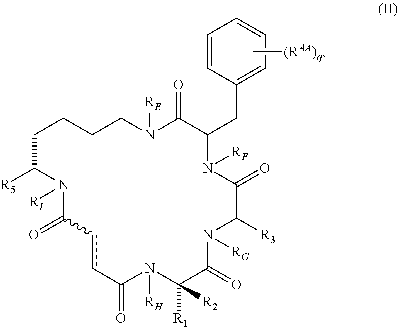

or pharmaceutically acceptable salts, solvates, hydrates, stereoisomers, polymorphs, tautomers, and isotopically enriched forms thereof,
wherein:

q is 0 or an integer between 1 and 5, inclusive;

[symbol] is a single or double C—C bond, wherein when [symbol] is a double C—C bond, then [symbol] indicates that the adjacent C—C double bond is in a cis or trans configuration; and

each instance of R₁, R₂, R₃, R₅, R_{E}, R_{F}, R_{G}, R_{H}, and R_{I} are as defined in Formula (I);

each instance of R^{AA} is independently halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A3}, —N(R^{A4})₂, —SR^{A3}, —C(═O)R^{A3}, —C(═O)OR^{A3}, —C(═O)SR^{A3}, —C(═O)N(R^{A4})₂, —OC(═O)R^{A3}, —OC(═O)OR^{A3}, —OC(═O)SR^{A3}, —OC(═O)N(R^{A4})₂, —NR^{A4}C(═O)R^{A4}, —NR^{A4}C(═O)OR^{A3}, —NR^{A4}C(═O)SR^{A3}, —NR^{A4}C(═O)N(R^{A4})₂, —SC(═O)R^{A3}, —SC(═O)OR^{A3}, —SC(═O)SR^{A3}, —SC(═O)N(R^{A4})₂, —C(═NR^{A4})R^{A3}, —C(═NR^{A4})OR^{A3}, —C(═NR^{A4})SR^{A3}, —C(═NR^{A4})N(R^{A4})₂, —OC(═NR^{A4})R^{A3}, —OC(═NR^{A4})OR^{A3}, —OC(═NR^{A4})SR^{A3}, —OC(═NR^{A4})N(R^{A4})₂, —NR^{A4}C(═NR^{A4})R^{A2}, —NR^{A4}C(═NR^{A4})OR^{A3}, —NR^{A4}C(═NR^{A4})SR^{A3}, —NR^{A4}C(═NR^{A4})N(R^{A4})₂, —SC(═NR^{A4})R^{A3}, —SC(═NR^{A4})OR^{A3}, —SC(═NR^{A4})SR^{A3}, —SC(═NR^{A4})N(R^{A4})₂, —C(═S)R^{A3}, —C(═S)OR^{A3}, —C(═S)SR^{A3}, —C(═S)N(R^{A4})₂, —OC(═S)R^{A3}, —OC(═S)OR^{A3}, —OC(═S)SR^{A3}, —OC(═S)N(R^{A4})₂, —NR^{A4}C(═S)R^{A4}, —NR^{A4}C(═S)OR^{A3}, —NR^{A4}C(═S)SR^{A3}, —NR^{A4}C(═S)N(R^{A4})₂, —SC(═S)R^{A3}, —SC(═S)OR^{A3}, —SC(═S)SR^{A3}, —SC(═S)N(R^{A4})₂, —S(═O)R^{A3}, —SO₂R^{A3}, —NR^{A4}SO₂R^{A3}, —SO₂N(R^{A4})₂, —N₃, —CN, —SCN, and —NO₂,

wherein each occurrence of R^{A3} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl; and each occurrence of R^{A4} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group, or two R^{A4} groups are joined to form a substituted or unsubstituted heterocyclic ring.

In some embodiments, the labeled IDE inhibitors are of Formula (III):

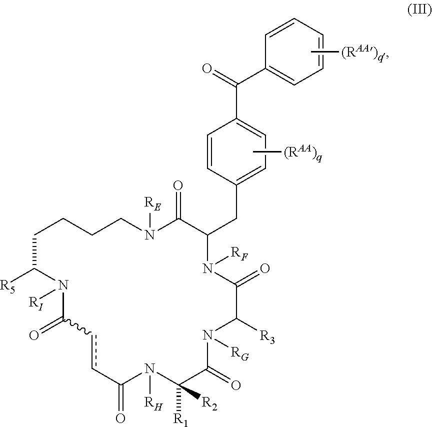

or pharmaceutically acceptable salts, solvates, hydrates, stereoisomers, polymorphs, tautomers, and isotopically enriched forms thereof,
wherein:

q is 0 or an integer between 1 and 5, inclusive;

q′ is 0 or an integer between 1 and 5, inclusive;

[symbol] is a single or double C—C bond, wherein when [symbol] is a double C—C bond, then [symbol] indicates that the adjacent C—C double bond is in the cis or trans configuration; and

each instance of R₁, R₂, R₃, R₅, R_{E}, R_{F}, R_{G}, R_{H}, and R_{I} are as defined in Formula (I);

each instance of R^{AA} is independently halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A3}, —N(R^{A4})₂, —SR^{A3}, —C(═O)R^{A3}, —C(═O)OR^{A3}, —C(═O)SR^{A3}, —C(═O)N(R^{A4})₂, —OC(═O)R^{A3}, —OC(═O)OR^{A3}, —OC(═O)SR^{A3}, —OC(═O)N(R^{A4})₂, —NR^{A4}C(═O)R^{A4}, —NR^{A4}C(═O)OR^{A3}, —NR^{A4}C(═O)SR^{A3}, —NR^{A4}C(═O)N(R^{A4})₂, —SC(═O)R^{A3}, —SC(═O)OR^{A3}, —SC(═O)SR^{A3}, —SC(═O)N(R^{A4})₂, —C(═NR^{A4})R^{A3}, —C(═NR^{A4})OR^{A3}, —C(═NR^{A4})SR^{A3}, —C(═NR^{A4})N(R^{A4})₂, —OC(═NR^{A4})R^{A3}, —OC(═NR^{A4})OR^{A3}, —OC(═NR^{A4})SR^{A3}, —OC(═NR^{A4})N(R^{A4})₂, —NR^{A4}C(═NR^{A4})R^{A2}, —NR^{A4}C(═NR^{A4})OR^{A3}, —NR^{A4}C(═NR^{A4})SR^{A3}, —NR^{A4}C(═NR^{A4})N(R^{A4})₂, —SC(═NR^{A4})R^{A3}, —SC(═NR^{A4})OR^{A3}, —SC(═NR^{A4})SR^{A3}, —SC(═NR^{A4})N(R^{A4})₂, —C(═S)R^{A3}, —C(═S)OR^{A3}, —C(═S)SR^{A3}, —C(═S)N(R^{A4})₂, —OC(═S)R^{A3}, —OC(═S)OR^{A3}, —OC(═S)SR^{A3}, —OC(═S)N(R^{A4})₂, —NR^{A4}C(═S)R^{A4}, —NR^{A4}C(═S)OR^{A3}, —NR^{A4}C(═S)SR^{A3}, —NR^{A4}C(═S)N(R^{A4})₂, —SC(═S)R^{A3}, —SC(═S)OR^{A3}, —SC(═S)SR^{A3}, —SC(═S)N(R^{A4})₂, —S(═O)R^{A3}, —SO₂R^{A3}, —NR^{A4}SO₂R^{A3}, —SO₂N(R^{A4})₂, —N₃, —CN, —SCN, and —NO₂,

wherein each occurrence of R^{A3} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl; and each occurrence of R^{A4} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group, or two R^{A4} groups are joined to form a substituted or unsubstituted heterocyclic ring;

each instance of R^{AA}′ is independently halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A3′}, —N(R^{A4′})₂, —SR^{A3′}, —C(═O)R^{A3′}, —C(═O)OR^{A3′}, —C(═O)SR^{A3′}, —C(═O)N(RA⁴)₂, —OC(═O)R^{A3′}, —OC(═O)OR^{A3′}, —OC(═O)SR^{A3′}, —OC(═O)N(R^{A4})₂, —NR^{A4′}C(═O)R^{A4′}, —NR^{A4′}C(═O)OR^{A3′}, —NR^{A4′}C(═O)SR^{A3′}, —NR^{A4′}C(═O)N(R^{A4})₂, —SC(═O)R^{A3′}, —SC(═O)OR^{A3′}, —SC(═O)SR^{A3′}, —SC(═O)N(R^{A4′})₂, —C(═NR^{A4′})R^{A3′}, —C(═NR^{A4′})OR^{A3′}, —C(═NR^{A4′})SR^{A3′}, —C(═NR^{A4′})N(R^{A4′})₂, —OC(═NR^{A4′})R^{A3′}, —OC(═NR^{A4′})OR^{A3′}, —OC(═NR^{A4′})SR^{A3′}, —OC(═NR^{A4′})N(R^{A4′})₂, —NR^{A4′}C(═NR^{A4′})R^{A3}, —NR^{A4′}C(═NR^{A4′})OR^{A3′}, —NR^{A4′}C(═NR^{A4′})SR^{A3′}, —NR^{A4′}C(═NR^{A4′}C(═NR^{A4′})N(R^{A4′})₂, —SC(═NR^{A4′})R^{A3′}, —SC(═NR^{A4′})OR^{A3′}, —SC(═NR^{A4′})SR^{A3′}, —SC(═NR^{A4′})N(R^{A4′})₂, —C(═S)R′, —C(═S)OR^{A3′}, —C(═S)SR^{A3′}, —C(═S)N(R^{A4′})₂, —OC(═S)R^{A3′}, —OC(═S)OR^{A3′}, —OC(═S)SR^{A3′}, —OC(═S)N(R^{A4′})₂, —NR^{A4′}C(═S)R^{A4′}, —NR^{A4′}C(═S)OR^{A3′}, —NR^{A4′}C(═S)SR^{A3′}, —NR^{A4′}C(═S)N(R^{A4′})₂, —SC(═S)R^{A3′}, —SC(═S)OR^{A3′}, —SC(═S)SR^{A3 ′}, —SC(═S)N(R^{A4′})₂, —S(═O)R^{A3′}, —SO₂R^{A3′}, —NR^{A4′}SO₂R^{A3′}, —SO₂N(R^{A4′})₂, —N₃, —CN, —SCN, and —NO₂,

wherein each occurrence of R^{A3′} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl; and each occurrence of R^{A4′} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group, or two R^{A4′} groups are joined to form a substituted or unsubstituted heterocyclic ring.

In some embodiments, the labeled IDE inhibitors provided herein are of Formula (IV):

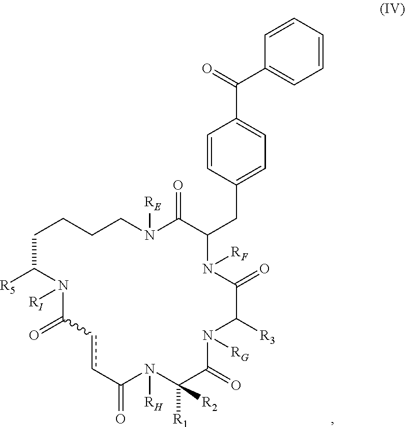

or pharmaceutically acceptable salts, solvates, hydrates, stereoisomers, polymorphs, tautomers, and isotopically enriched forms thereof,
wherein each instance of R₁, R₂, R₃, R₅, R_{E}, R_{F}, R_{G}, R_{H}, and R_{I} are as defined in Formula (I). In certain embodiments of Formula (IV), R₁ represents —H, —CH₃, —CH₂—CH₂—C(═O)—NH₂, —CH₂—CH₂—CH₂—NH—C(═NH)—NH₂, —(CH₂)ₚ-cyclohexyl, —(CH₂)ₚ-cyclopentyl, —(CH₂)ₚ-cyclobutyl, —(CH₂)ₚ-cyclopropyl, —(CH₂)ₚ-phenyl, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{K}, —N(R^{L})₂, —SR^{K}, —C(═O)R^{K}, —C(═O)OR^{K}, —C(═O)SR^{K}, —C(═O)N(R^{L})₂, —OC(═O)R^{K}, —OC(═O)OR^{K}, —OC(═O)SR^{K}, —OC(═O)N(R^{L})₂, —NR^{L}C(═O)R^{L}, —NR^{L}C(═O)OR^{K}, —NR^{L}C(═O)SR^{K}, —NR^{L}C(═O)N(R^{L})₂, —SC(═O)R^{K}, —SC(═O)OR^{K}, —SC(═O)SR^{K}, —SC(═O)N(R^{L})₂, —C(═NR^{L})R^{K}, —C(═NR^{L})OR^{K}, —C(═NR^{L})SR^{K}, —C(═NR^{L})N(R^{L})₂, —OC(═NR^{L})R^{K}, —OC(═NR^{L})OR^{K}, —OC(═NR^{L})SR^{K}, —OC(═NR^{L})N(R^{L})₂, —NR^{L}C(═NR^{L})R^{A3}, —NR¹C(═NR^{L})OR^{K}, —NR^{L}C(═NR^{L})SR^{K}, —NR^{L}C(═NR^{L})N(R^{L})₂, —SC(═NR^{L})R^{K}, —SC(═NR^{L})OR^{K}, —SC(═NR^{L})SR^{K}, —SC(═NR^{L})N(R^{L})₂, —C(═S)R^{K}, —C(═S)OR^{K}, —C(═S)SR^{K}, —C(═S)N(R^{L})₂, —OC(═S)R^{K}, —OC(═S)OR^{K}, —OC(═S)SR^{K}, —OC(═S)N(R^{L})₂, —NR^{L}C(═S)R^{L}, —NR^{L}C(═S)OR^{K}, —NR^{L}C(═S)SR^{K}, —NR^{L}C(═S)N(R^{L})₂, —SC(═S)R^{K}, —SC(═S)OR^{K}, —SC(═S)SR^{K}, —SC(═S)N(R¹)₂, —S(═O)R^{K}, —SO₂R^{K}, —NR^{L}SO₂R^{K}, —SO₂N(R^{L})₂, —N₃, —CN, —SCN, and —NO₂,

wherein each occurrence of R^{K} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl; and each occurrence of R^{L} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl; or a nitrogen protecting group; and each occurrence of p is independently 0 or an integer between 1 and 10 inclusive;

R₂ represents —H or —(CH₂)_{q}—CH₃, wherein q is 0 or an integer between 1 and 10 inclusive;

R₃ represents —(CH₂)ᵣ-cyclohexyl, —(CH₂)ᵣ-cyclopentyl, —(CH₂)ᵣ-cyclobutyl, —(CH₂)ᵣ-cyclopropyl, —(CH₂)ᵣ-phenyl, or (CH₂)ᵣ-R_{z}, wherein r is independently 0 or an integer between 1 and 10 inclusive, and wherein R_{z} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl; either in linear or cyclic form;

R₅ comprises a detectable label and, optionally, a linker; and [symbol] is a double C—C bond, in either the cis or trans configuration.

In some embodiments, the labeled IDE inhibitory compounds provided herein are of formula (V):

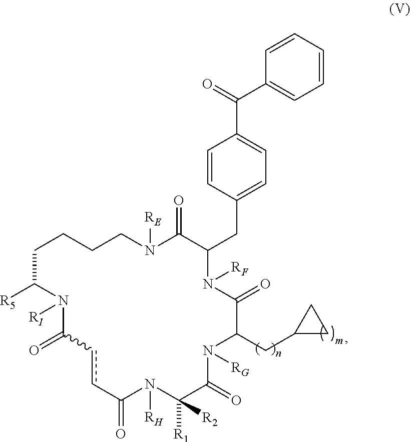

or pharmaceutically acceptable salts, solvates, hydrates, stereoisomers, polymorphs, tautomers, isotopically enriched forms, and prodrugs thereof, wherein each instance of R₁, R₂, R₅, R_{E}, R_{F}, R_{G}, R_{H}, and R_{I} are as defined in Formula (I). In certain embodiments of Formula (V), R₁ represents —H, —CH₃, —CH₂—CH₂—C(═O)—NH₂, —CH₂—CH₂—CH₂—NH—C(═NH)—NH₂, —(CH₂)ₚ-cyclohexyl, —(CH₂)ₚ-cyclopentyl, —(CH₂)ₚ-cyclobutyl, —(CH₂)ₚ-cyclopropyl, —(CH₂)ₚ-phenyl, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{K}, N(R^{L})₂ , SR^{K}, —C(═O)R^{K}, —C(═O)OR^{K}, —C(═O)SR^{K}, —C(═O)N(R^{L})₂, —OC(═O)R^{K}, —OC(═O)OR^{K}, —OC(═O)SR^{K}, —OC(═O)N(R^{L})₂, —NR^{L}C(═O)R^{L}, —NR^{L}C(═O)OR^{K}, —NR^{L}C(═O)SR^{K}, —NR^{L}C(═O)N(R^{L})₂, —SC(═O)R^{K}, —SC(═O)OR^{K}, —SC(═O)SR^{K}, —SC(═O)N(R^{L})₂, —C(═NR^{L})R^{K}, —C(═NR^{L})OR^{K}, —C(═NR^{L})SR^{K}, —C(═NR^{L})N(R^{L})₂, —OC(═NR^{L})R^{K}, —OC(═NR^{L})OR^{K}, —OC(═NR^{L})SR^{K}, —OC(═NR^{L})N(R^{L})₂, —NR^{L}C(═NR^{L})R^{A3}, —NR^{L}C(═NR^{L})OR^{K}, —NR^{L}C(═NR^{L})SR^{K}, —NR^{L}C(═NR^{L})N(R^{L})₂, —SC(═NR^{L})R^{A3}, —SC(═NR^{L})OR^{K}, —SC(═NR^{L})SR^{K}, —SC(═NR^{L})N(R^{L})₂, —C(═S)R^{K}, —C(═S)OR^{K}, —C(═S)SR^{K}, —C(═S)N(R^{L})₂, —OC(═S)R^{K}, —OC(═S)OR^{K}, —OC(═S)SR^{K}, —OC(═S)N(R^{L})₂, —NR^{L}C(═S)R^{L}, —NR^{L}C(═S)OR^{K}, —NR^{L}C(═S)SR^{K}, —NR^{L}C(═S)N(R^{L})₂, —SC(═S)R^{K}, —SC(═S)OR^{K}, ═SC(═S)SR^{K}, l SC(═S)N(R^{L})₂, —S(═O)R^{K}, —SO₂R^{K}, —NR^{L}SO₂R^{K}, —SO₂N(R^{L})₂, —N₃, —CN, —SCN, and —NO₂,

wherein each occurrence of R^{K} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl; and each occurrence of R^{L} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl; or a nitrogen protecting group; and each occurrence of p is independently 0 or an integer between 1 and 10 inclusive;

R₂ represents —H or —(CH₂)_{q}—CH₃, wherein q is 0 or an integer between 1 and 10 inclusive;

R₅ comprises a detectable label and, optionally, a linker; and; and wherein

each occurrence of n is independently 0 or an integer between 1 and 10 inclusive,

each occurrence of m is independently an integer between 1 and 5 inclusive; and [symbol] is a double C—C bond, in either the cis or trans configuration.

In some embodiments, the labeled macrocyclic IDE inhibitors provided herein are trans-olefins of formula (V), as provided by formula (VI):

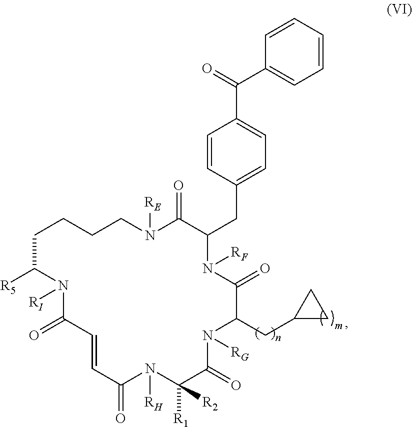

or pharmaceutically acceptable salts, solvates, hydrates, stereoisomers, polymorphs, tautomers, isotopically enriched forms, and prodrugs thereof, wherein each instance of R₁, R₂, R_{E}, R_{F}, R_{G}, R_{H}, and R_{I} are as defined in Formula (I). In certain embodiments of Formula (VI), R₁ represents —H, —CH₃, —CH₂—CH₂—C(═O)—NH₂, —CH₂—CH₂—CH₂—NH—C(═NH)—NH₂, —(CH₂)ₚ-cyclohexyl, —(CH₂)ₚ-cyclopentyl, —(CH₂)ₚ-cyclobutyl, —(CH₂)ₚ-cyclopropyl, —(CH₂)ₚ-phenyl, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{K}, —N(R^{L})₂, —SR^{K}, —C(═O)R^{K}, —C(═O)OR^{K}, —C(═O)SR^{K}, —C(═O)N(R^{L})₂, —OC(═O)R^{K}, —OC(═O)OR^{K}, —OC(═O)SR^{K}, —OC(═O)N(R^{L})₂, —NR^{L}C(═O)R^{L}, —NR^{L}C(═O)OR^{K}, —NR^{L}C(═O)SR^{K}, —NR^{L}C(═O)N(R^{L})₂, —SC(═O)R^{K}, —SC(═O)OR^{K}, —SC(═O)SR^{K}, —SC(═O)N(R^{L})₂, —C(═NR^{L})R^{K}, —C(═NR^{L})OR^{K}, —C(═NR^{L})SR^{K}, —C(═NR^{L})N(R^{L})₂, —OC(═NR^{L})R^{K}, —OC(═NR^{L})OR^{K}, —OC(═NR^{L})SR^{K}, —OC(═NR^{L})N(R^{L})₂, —NR^{L}C(═NR^{L})R^{A3}, —NR^{L}C(═NR^{L})OR^{K}, —NR^{L}C(═NR^{L})SR^{K}, —NR^{L}C(═NR^{L})N(R^{L})₂, —SC(═NR^{L})R^{K}, —SC(═NR^{L})OR^{K}, —SC(═NR^{L})SR^{K}, —SC(═NR^{L})N(R^{L})₂, —C(═S)R^{K}, —C(═S)OR^{K}, —C(═S)SR^{K}, —C(═S)N(R^{L})₂, —OC(═S)R^{K}, —OC(═S)OR^{K}, —OC(═S)SR^{K}, —OC(═S)N(R^{L})₂, —NR^{L}C(═S)R^{L}, —NR^{L}C(═S)OR^{K}, —NR^{L}C(═S)SR^{K}, —NR^{L}C(═S)N(R^{L})₂, —SC(═S)R^{K}, —SC(═S)OR^{K}, —SC(═S)SR^{K}, —SC(═S)N(R^{L})₂, —S(═O)R^{K}, —SO₂R^{K}, —NR^{L}SO₂R^{K}, —SO₂N(R^{L})₂, N₃, —CN, —SCN, and —NO₂,

wherein each occurrence of R^{K} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl; and each occurrence of R^{L} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl; or a nitrogen protecting group; and each occurrence of p is independently 0 or an integer between 1 and 10 inclusive;

R₂ represents —H or —(CH₂)_{q}—CH₃, wherein q is 0 or an integer between 1 and 10 inclusive;

R₅ comprises a detectable label and, optionally, a linker; and; and wherein

n is 0 or an integer between 1 and 10 inclusive,

m is an integer between 1 and 5 inclusive; and [symbol] is a double C—C bond, in either a cis or trans configuration.

In some embodiments, the macrocyclic IDE inhibitors provided herein include a C═C double bond in the macrocycle backbone. The position of this double bond is provided as [symbol] in Formula (I)-(V), and as ═ in Formula (VI)-(VIII). In some embodiments, the macrocycle backbone C═C double bond is in the cis-configuration. The respective macrocycles are also referred to herein as cis-olefins. In some embodiments, the macrocycle backbone C═C double bond is in the trans-olefin configuration. The respective macrocycles are also referred to herein as trans-olefins.

In some embodiments, a labeled macrocyclic IDE inhibitor described herein, for example, macrocycle 6b, is provided as a cis-olefin, without any significant or any detectable amount of the respective trans-olefin isomer. In some embodiments, an IDE inhibitor described herein, for example, macrocycle 6b, is provided as a trans-olefin, without any significant or any detectable amount of the respective cis-olefin isomer. In some embodiments, an IDE inhibitor described herein is provided as a mixture of cis-olefin and trans-olefin isomers. Methods for the synthesis of the IDE inhibitors described herein in the cis- or trans-configuration are described in PCT application PCT/US2012/044977, entitled Macrocyclic Insulin-Degrading Enzyme (IDE) Inhibitors and Uses Thereof, the entire contents of which are incorporated herein by reference. Additional methods useful for the synthesis and production of cis- and trans-olefins that are useful for the generation of the labeled macrocyclic IDE inhibitors disclosed herein are known to those of skill in the art, and the invention is not limited in this respect.

In some embodiments, the labeled IDE-inhibitor is of any of Formula (I)-(VI), wherein n is 1 and/or m is 4. In some embodiments, R₁ represents —H, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —CH₃, —CH₂—CH₂—C(═O)—NH₂, —CH₂—CH₂—CH₂—NH—C(═NH)—NH₂, —(CH₂)ₚ-cyclohexyl, —(CH₂)ₚ-cyclopentyl, —(CH₂)ₚ-cyclobutyl, —(CH₂)ₚ-cyclopropyl, —(CH₂)ₚ-phenyl, —OR^{K}, —N(R^{L})₂, —SR^{K}, —C(═O)R^{K}, —C(═O)OR^{K}, —C(═O)SR^{K}, —C(═O)N(R^{L})₂, —OC(═O)R^{K}, —OC(═O)OR^{K}, —OC(═O)SR^{K}, —OC(═O)N(R^{L})₂, —NR^{L}C(═O)R^{L}, —NR^{L}C(═O)OR^{K}, —NR^{L}C(═O)SR^{K}, —NR^{L}C(═O)N(R^{L})₂, —SC(═O)R^{K}, —SC(═O)OR^{K}, —SC(═O)SR^{K}, —SC(═O)N(R^{L})₂, —C(═NR^{L})R^{K}, —C(═NR^{L})OR^{K}, —C(═NR^{L})SR^{K}, —C(═NR^{L})N(R^{L})₂, —OC(═NR^{L})R^{K}, —OC(═NR^{L})OR^{K}, —OC(═NR^{L})SR^{K}, —OC(═NR^{L})N(R^{L})₂, —NR^{L}C(═NR^{L})R^{A3}, —NR^{L}C(═NR^{L})OR^{K}, —NR^{L}C(═NR^{L})SR^{K}, —NR^{L}C(═NR^{L})N(R^{L})₂, —SC(═NR^{L})R^{K}, —SC(═NR^{L})OR^{K}, —SC(═NR^{L})SR^{K}, —SC(═NR^{L})N(R^{L})₂, —C(═S)R^{K}, —C(═S)OR^{K}, —C(═S)SR^{K}, —C(═S)N(R^{L})₂, —OC(═S)R^{K}, —OC(═S)OR^{K}, —OC(═S)SR^{K}, —OC(═S)N(R^{L})₂, —NR^{L}C(═S)R^{L}, —NR^{L}C(═S)OR^{K}, —NR^{L}C(═S)SR^{K}, —NR^{L}C(═S)N(R^{L})₂, —SC(═S)R^{K}, —SC(═S)OR^{K}, —SC(═S)SR^{K}, —SC(═S)N(R^{L})₂, —S(═O)R^{K}, —SO₂R^{K}, —NR^{L}SO₂R^{K}, —SO₂N(R^{L})₂, —N₃, —CN, —SCN, and —NO₂,wherein each occurrence of R^{K} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl; and each occurrence of R^{L} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl; or a nitrogen protecting group; and each occurrence of p is independently 0 or an integer between 1 and 10 inclusive. In some embodiments, R₁ represents —H, —CH₃, —CH₂—CH₂—C(═O)—NH₂, —CH₂—CH₂—CH₂—NH—C(═NH)-NH₂, —(CH₂)ₚ-cyclohexyl, —(CH₂)ₚ-cyclopentyl, —(CH₂)ₚ-cyclobutyl, —(CH₂)ₚ-cyclopropyl, —(CH₂)ₚ-phenyl, wherein each occurrence of p is independently 0 or an integer between 1 and 10 inclusive. In some embodiments, R₁ represents —H, —CH₃, —CH₂—CH₂—C(═O)—NH₂, —CH₂—CH₂—CH₂—NH—C(═NH)—NH₂, —CH₂-cyclohexyl, or —CH₂-cyclopropyl. In some embodiments, R₂ represents —H or —(CH₂)_{q}-CH₃, wherein q is 0 or an integer between 1 and 10 inclusive.

In some embodiments, the labeled IDE-inhibitor comprises a macrocycle structure selected from the group consisting of 1b, 2b, 3b, 4b, 5n, 6a, 6c, 6b, 6bk, and 7-29, wherein the macrocyclic structure is conjugated to a detectable label. In some embodiments, the labeled IDE-inhibitor comprises macrocycle 6bk conjugated to a detectable label. In some embodiments, the detectable label comprises a fluorophore or a detectable isotope. In some embodiments, R₅ comprises fluorescein. In some embodiments, R₅ comprises a linker. In some embodiments, the compound is of Formula (31).

In some embodiments, the detectable label comprises a binding agent, e.g., a molecule or moiety that binds to another molecule with high affinity. In some embodiments, the binding agent is an antibody or an antigen-binding antibody fragment, a nanobody, an ScFv, an aptamer, or an adnectin. In some embodiments, the binding agent is a ligand, for example, biotin, polyhistidine, or FK506. Other binding agents are known to those of skill in the art and the invention is not limited in this respect. In some embodiments, the binding agent specifically binds an antigen, for example, an antigen immobilized on a solid surface or a cellular antigen, e.g., a cell-surface antigen. In some embodiments, the binding is through non-covalent interaction. In some embodiments, the binding is specific, meaning that the binding agent binds only one particular type of molecule, or a narrow class of highly similar molecules with high affinity. Non-limiting examples of binding agents are antibodies, antibody fragments, ligands, receptors, aptamers, and adnectins. In some embodiments, wherein R₅ comprises a linker. In some embodiments, the compound is of Formula (30).

The disclosure also embraces pharmaceutically acceptable salts of the macrocyclic IDE inhibitor disclosed herein, whether conjugated to a detectable label or not, as well as pharmaceutical compositions comprising the IDE inhibitors disclosed herein, or a pharmaceutically acceptable salt thereof.

### Clinical Applications of Macrocyclic IDE Inhibitors

The data provided herein show that small-molecule IDE inhibitors can improve oral glucose tolerance to an extent comparable to that of DPP4 inhibitors. These data are relevant to human clinical applications, as evidenced by the repeated recognition of IDE as a diabetes susceptibility gene in humans.⁵⁻⁹ Additional in vivo and biochemical experiments described herein using 6bK led to the surprising discovery that IDE regulates the stability and signaling of glucagon and amylin, in addition to its established role in insulin degradation¹⁰⁻¹². The identification of these two additional pancreatic hormones as endogenous IDE substrates advances our understanding of the role of IDE in regulating physiological glucose homeostasis (see FIG. 6). Amylin-mediated effects on gastric emptying and satiety during meals have been recently recognized to have therapeutic relevance in the treatment of diabetes,^{2,31} and the results presented herein represent the first demonstration of a small molecule that can regulate amylin signaling. Moreover, the link between IDE and glucagon revealed herein provides additional evidence of the importance of glucagon regulation in human diabetes.⁴⁹

The data described herein reveals a specific pharmacological requirement for therapeutic IDE inhibition, namely, that transient, rather than chronic, IDE inhibition is desirable to prevent elevation of glucagon signaling in some embodiments⁴⁹ (see FIG. 6). Based on this discovery, an anti-diabetic therapeutic strategy was developed that includes the use of a fast-acting IDE inhibitor that can be taken with a meal to transiently augment endogenous insulin and amylin responses to help control post-prandial glycaemia^{31,32}, and that is cleared or degraded before glucagon secretion resumes. Similar pre-meal therapeutic strategies with short-lived agents have already proven successful with fast-acting insulin analogs, secretagogues, and amylin supplementation.^{32,33} Some aspects of this invention are based on the recognition that such agents have synergistic effects when co-administered with an IDE inhibitor^{3,50}. Additionally, the combination of an IDE inhibitor with incretin therapy^{2,31} or a glucagon receptor antagonist⁴⁹, is also recognized to be therapeutic, as evidenced by the experiments with glucagon-receptor deficient mice described herein.

Some aspects of this disclosure provide methods of administering an IDE inhibitor to a subject in order to improve insulin signaling. In some embodiments, these methods are useful for improving oral glucose tolerance, to modulate gastric emptying, and to modulate satiety during meals in a subject, for example, in a subject having diabetes. In some embodiments, the method involves administering an IDE inhibitor to a subject according to a dosing schedule that results in transient IDE inhibition, e.g., in temporal proximity to the intake of a meal. In some embodiments, the IDE inhibitor is administered according to a dosing schedule that results in transient IDE inhibition, but avoids or minimizes an elevation of glucagon signaling. In some embodiments, a method is provided that comprises administering an IDE inhibitor provided herein together with an additional therapeutic agent, e.g., with a pre-meal therapeutic agent, such as, for example, a fast-acting insulin analog, secretagogue (e.g., a substance that causes another substance to be secreted), amylin supplement, incretin therapy, or a glucagon receptor antagonist. Additional diabetes therapeutics are known to those of skill in the art and the disclosure is not limited in this respect. In some such embodiments, the IDE inhibitor and the additional therapeutic agent are in an amount that is individually effective to elicit the desired effect in a subject, e.g., the desired level and time period of transient IDE inhibition and the desired therapeutic effect of the additional agent. In some embodiments, the IDE inhibitor and the additional agent are administered at a dose that, by itself, would not result in a therapeutic effect, but that, in combination, results in a desired therapeutic effect.

In some embodiments, transient inhibition of IDE according to the methods provided herein results in a stabilization (e.g., greater half-life) of insulin and in improved (e.g., increased) insulin signaling without elevating glucagon signaling. Accordingly, the in vivo methods of using the macrocyclic IDE inhibitors provided herein are useful in improving insulin signaling in subjects having a disease associated with IDE activity, or impaired insulin signaling, for example, in patients exhibiting metabolic syndrome or diabetes (e.g., Type I or Type II diabetes) while avoiding the negative effects of increased glucagon signaling that chronic IDE inhibition may effect.

Some aspects of this disclosure relate to the surprising discovery that, in some embodiments, administration of the IDE inhibitors provided herein results in modulation of CGRP signaling in a subject. Accordingly, some embodiments of this disclosure provide methods comprising administering an IDE inhibitor, e.g., a macrocyclic IDE inhibitor as described herein, to a subject in an amount effective to modulate CGRP signaling in the subject. Some aspects of this disclosure relate to the surprising discovery that, in some embodiments, administration of the IDE inhibitors provided herein results in modulation, e.g., a decrease, of the blood pressure of a subject, or in modulation, e.g., a decrease, of the heart rate of a subject. Accordingly, some embodiments of this disclosure provide methods comprising administering an IDE inhibitor, e.g., a macrocyclic IDE inhibitor as described herein, to a subject in an amount effective to modulate the blood pressure or the heart rate in the subject. In some embodiments, the IDE inhibitor is administered according to a dosing schedule resulting in transient IDE inhibition, e.g., as described in more detail elsewhere herein. In some embodiments, the IDE inhibitor is administered in an amount effective to decrease the blood pressure of the subject as compared to a baseline or pre-treatment blood pressure.

In some embodiments, the IDE inhibitor is administered in an amount effective to decrease the blood pressure of the subject at least 1%, at least 2%, at least 2.5%, at least 3%, at least 4%, at least 5%, at least 6%, at least 7%, at least 8%, at least 9%, at least 10%, at least 12.5%, at least 15%, at least 20%, at least 25%, or at least 30%, as compared to a baseline or pre-treatment blood pressure. In some embodiments, the IDE inhibitor is administered in an amount effective to decrease the heart rate in the subject as compared to a baseline or pre-treatment blood pressure. In some embodiments, the IDE inhibitor is administered in an amount effective to decrease the heart rate of the subject at least 1%, at least 2%, at least 2.5%, at least 3%, at least 4%, at least 5%, at least 6%, at least 7%, at least 8%, at least 9%, at least 10%, at least 12.5%, at least 15%, at least 20%, at least 25%, or at least 30%, as compared to a baseline or pre-treatment blood pressure. In some embodiments, the IDE inhibitor is administered in an amount effective to decrease the blood pressure and/or the heart rate for a time period of at least 30 minutes, at least 1 hour, at least 2 hours, at least 3 hours, at least 4 hours, at least 5 hours, at least 6 hours, at least 12 hours, less than 30 minutes, less than 1 hour, less than 2 hours, less than 3 hours, less than 4 hours, less than 5 hours, less than 6 hours, les than 12 hours, or less than 24 hours, or any possible combination thereof, e.g., of at least 6 hours and less than 12 hours, at least 12 hours and less than 24 hours.

In some embodiments, the method further comprises determining the stability and/or the level of signaling of CGRP in the subject. In some embodiments, the method further comprises determining the blood pressure, and/or the heart rate in the subject.

In some embodiments, the in vitro or in vivo methods of transiently inhibiting the activity of IDE comprise contacting an IDE with an IDE inhibitor provided herein in an amount effective to inhibit the activity of IDE for a period of less than 6 hours. In some embodiments, the IDE inhibitor is administered to a subject in an amount effective to inhibit the activity of IDE for a period of less than 5 hours, less than 4 hours, less than 3 hours, less than 2 hours, or less than 1 hour. In some embodiments, the IDE inhibitor is administered to a subject in an amount effective to inhibit the activity of IDE for a time period equal or shorter to the time period in which the blood glucose concentration reaches a baseline level in the subject after a meal. In some embodiments, the IDE inhibitor is administered to a subject in an amount effective to inhibit the activity of IDE for a time period equal or shorter to the time period in which the blood glucose concentration reaches a fasting level in the subject after a meal. In some embodiments, a fasting level is the level of glucose observed in the subject after 8-10 hours of fasting. In some embodiments, the fasting level is lower than the baseline level. In some embodiments, the IDE inhibitor is administered to a subject in an amount effective to transiently inhibit the activity of IDE for a time period equal or shorter to the time period in which the blood glucose concentration drops below 250 mg/dl, 240 mg/dl, 230 mg/dl, 220 mg/dl, 210 mg/dl, 200 mg/dl, 190 mg/dl, 180 mg/dl, 170 mg/dl, 160 mg/dl, 150 mg/dl, 140 mg/dl, 130 mg/dl, or 120 mg/dl in the subject after a meal. In some embodiments, the IDE inhibitor is administered to a subject in an amount effective to transiently inhibit the activity of IDE for a time period equal or shorter to the time period in which the blood glucose concentration drops below 140 mg/dl (7.7 mmol/L) in the subject after a meal.

In some embodiments, inhibiting IDE activity refers to decreasing the activity of IDE, e.g., significantly or statistically significantly decreasing IDE activity, as compared to IDE activity in the absence of the IDE inhibitor. In some embodiments, inhibiting IDE activity refers to decreasing IDE activity to less than about 50%, less than about 25%, less than about 20%, less than about 10%, less than about 9%, less than about 8%, less than about 7%, less than about 6%, less than about 5%, less than about 4%, less than about 3%, less than about 2%, less than about 1%, less than about 0.1%, less than about 0.01%, or less than about 0.001% of IDE activity observed or expected in the absence of an IDE-inhibitory compound. In some embodiments, an in vivo method of transiently inhibiting IDE is provided that comprises administering an IDE inhibitor provided herein, or a pharmaceutically acceptable composition thereof, to a subject in an amount effective to reduce IDE activity in the subject to less than about 75%, less than about 50%, less than about 25%, less than about 20%, less than about 10%, less than about 9%, less than about 8%, less than about 7%, less than about 6%, less than about 5%, less than about 4%, less than about 3%, less than about 2%, less than about 1%, less than about 0.1%, less than about 0.01%, or less than about 0.001% of the IDE activity as compared to the IDE activity in the absence of the compound.

In some embodiments, the IDE inhibitor is administered to the subject in temporal proximity to the subject eating a meal. In some embodiments, the IDE inhibitor is administered within 2 hours before the meal, within 1 hour before the meal, within 30 minutes before the meal, within 15 minutes before the meal, within 10 minutes before the meal, within 5 minutes before the meal, within 1 minute before the meal, immediately before the meal, during the meal, at the end of the meal, immediately after the meal, within 1 minute after the meal, within 5 minutes after the meal, within 10 minutes after the meal, within 15 minutes after the meal, within 30 minutes after the meal, or within 1 hour after the meal. In some embodiments, the IDE inhibitor is administered to the subject once a day, twice a day, three times a day, four times a day, or five or more times a day in temporal proximity of a meal. In some embodiments, the IDE inhibitor is administered to the subject in temporal proximity to each meal the subject takes in. In some embodiments, if the subject skips a meal, the administration of the IDE inhibitor is also skipped.

The amount of a macrocyclic IDE inhibitor as described herein that is required for effective transient inhibition of IDE in a subject, or for the treatment or amelioration of a disease associated with IDE activity, such as diabetes, will vary from subject to subject, depending on a variety of factors, including, for example, the disorder being treated and the severity of the disorder, or the level of IDE activity in the subject, the activity of the specific macrocyclic IDE inhibitor administered, the specific composition employed; the age, body weight, general health, sex, and diet of the patient; the time of administration, route of administration, and rate of excretion of the specific compound employed; the duration of the treatment; drugs used in combination or coincidental with the specific compound employed; and like factors well known in the medical arts. The macrocyclic IDE inhibitor described herein are preferably formulated in dosage unit form for ease of administration and uniformity of dosage. Oral dosage forms are preferred. It will be understood that in some embodiments involving administration of a macrocyclic IDE inhibitor described herein to a human patient, the total daily dose may be determined by the attending physician based on sound medical judgment.

In some embodiments, a macrocyclic IDE inhibitor described herein is formulated into a pharmaceutically acceptable composition comprising the IDE inhibitor, or a pharmaceutically acceptable salt thereof, and optionally a pharmaceutically acceptable carrier. In some embodiments, the composition further comprises an additional therapeutic compound, for example, an additional diabetic therapeutic as described elsewhere herein. In some embodiments, after formulation with an appropriate pharmaceutically acceptable carrier of a desired dosage, the pharmaceutical composition can be administered to a subject, for example, a human subject via any suitable route, for example, orally, rectally, parenterally, intracisternally, intravaginally, intraperitoneally, topically (as by powders, ointments, or drops), bucally, as an oral or nasal spray, or the like. Oral administration is preferred.

In some embodiments, a macrocyclic IDE inhibitor described herein or identified by the methods provided herein, for example, in any of Formula (I)-(VIII), is administered to a subject, for example, orally or parenterally, at a dosage level of about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, and from about 1 mg/kg to about 25 mg/kg of the subject's body weight per day, or per meal in some embodiments where the administration is in temporal proximity of a meal, to obtain the desired therapeutic effect or the desired level of transient IDE inhibition.

Solid dosage forms for oral administration include capsules, tablets, pills, powders, and granules. In such solid dosage forms, a macrocyclic IDE inhibitor described herein is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

The macrocyclic IDE inhibitor described herein can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active protein may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Liquid dosage forms of the macrocyclic IDE inhibitor described herein, for example, for oral and parenteral administration include, but are not limited to, pharmaceutically acceptable emulsions, microemulsions, solutions, suspensions, syrups, and elixirs. In addition to the active compounds, the liquid dosage forms may contain inert diluents commonly used in the art, such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. In certain embodiments for parenteral administration, the compounds of the invention are mixed with solubilizing agents such polyethoxylated castor oil, alcohols, oils, modified oils, glycols, polysorbates, cyclodextrins, polymers, and combinations thereof.

Injectable preparations of the macrocyclic IDE inhibitor described herein, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

It will also be appreciated that the macrocyclic IDE inhibitors described herein and pharmaceutical compositions thereof can be employed in combination therapies, that is, the IDE inhibitors and pharmaceutical compositions provided herein can be administered concurrently with, prior to, or subsequent to, one or more other desired therapeutics or medical procedures. For example, in the context of metabolic syndrome or diabetes, a patient may receive a macrocyclic IDE inhibitor described herein and, additionally, a drug or pharmaceutical composition approved for the treatment of or commonly used to ameliorate a symptom associated with metabolic syndrome or diabetes. Similarly, if an IDE inhibitor or a pharmaceutical composition as provided herein is administered to a subject suffering from another disease, for example, from Alzheimer's Disease, the subject may receive a macrocyclic IDE inhibitor described herein and, additionally, a drug or pharmaceutical composition approved for the treatment of or commonly used to ameliorate a symptom associated with Alzheimer's disease. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a macrocyclic IDE inhibitor may be administered concurrently with another agent), or they may achieve different effects (e.g., control of any adverse effects).

In some embodiments, an IDE inhibitory macrocyclic compound provided herein is administered to a subject, for example, in the form of a pharmaceutically acceptable salt or as part of a pharmaceutical composition. In some embodiments, the subject is human. In some embodiments, the subject is an animal, for example, an experimental animal, e.g., an animal model of diabetes. In some embodiments, the animal is a mammal, for example, a rodent (e.g., a mouse, a rat, a hamster), a dog, a cat, a cow, a goat, a sheep, or a horse.

Other aspects of this invention provide methods of using a macrocyclic IDE inhibitor as described herein in the production of pharmaceutical compositions, or in the manufacture of a medicament, for the transient reduction of IDE activity. Some aspects of this invention provide methods of using a macrocyclic IDE inhibitor as described herein in the production of a pharmaceutical composition, or in the manufacture of a medicament, for the treatment, prophylaxis, and/or amelioration of a disease or disorder associated with aberrant IDE activity, impaired insulin signaling, or insulin resistance, for example, diabetes, or metabolic syndrome. In some embodiments, the pharmaceutical composition or the medicament is for the treatment, prophylaxis, and/or amelioration of a disease or disorder associated with aberrant IDE activity, impaired insulin signaling, or insulin resistance, for example, diabetes, or metabolic syndrome, wherein the disease or disorder is exhibited by a subject also exhibiting one or more symptoms of Alzheimer's disease. Some aspects of this invention relate to the use of a macrocyclic IDE inhibitor as described herein for the production of pharmaceutical compositions which can be used for treating, preventing, or ameliorating diseases responsive to the inhibition of IDE activity, for example, diabetes or metabolic syndrome.

### Kits

Some aspects of this disclosure provide a pharmaceutical pack or kit comprising one or more containers filled with a dosage form of an IDE inhibitor of formula VII or VIII; and instructions for administering the IDE inhibitor to a subject to achieve transient IDE inhibition. In some embodiments, the pack or kit may also include an additional therapeutic agent for use as a combination therapy together with the IDE inhibitor.

Some aspects of this disclosure provide kits for comprising an IDE-binding probe comprising an IDE-inhibitor conjugated to a detectable label; and instructions for performing an assay for identifying an IDE-binding compound. In some embodiments, the detectable label is a fluorophore and the instructions are for performing a fluorescence polarization assay.

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXAMPLES

### Example 1

### Anti-Diabetic Activity of Insulin-Degrading Enzyme Inhibitors Mediated by Multiple Hormones

The dynamic interplay between the production and proteolytic degradation of peptide hormones is a key mechanism underlying the regulation of human metabolism. Inhibition of the peptidases and proteases that degrade these hormones can elevate their effective concentrations and augment signaling. In some cases, the resulting insights can lead to the development of novel therapeutics¹. Dipeptidyl peptidase 4 (DPP4) inhibitors, for example, are anti-diabetic drugs that increase the concentration of the insulin-stimulating hormone glucagon-like peptide 1 (GLP-1), resulting in elevated insulin concentrations and lower blood glucose levels². Researchers have speculated for at least six decades that insulin signaling could also be augmented to improve glucose tolerance by inhibiting its degradation^{3,4}. The zinc metalloprotease insulin-degrading enzyme (IDE) is thought to be the primary enzyme responsible for inactivation of insulin in the liver and kidney^{3,4}. Recently, genome-wide association studies have identified predisposing and protective variants of the IDE locus linked to type-2 diabetes (T2D)⁵⁻⁹, suggesting a functional connection between IDE and glucose regulation in humans.

Based on the known biochemistry of IDE, inhibition of this enzyme is expected to elevate insulin levels and augment the response to glucose^{3,4}. While mice lacking a functional IDE gene (IDE^{-/-} mice) have elevated insulin levels, counterintuitively these animals exhibit impaired, rather than improved, glucose tolerance^{10,11} . Physiological studies with IDE^{-/-} mice concluded that chronic elevation of insulin in these animals results in a compensatory lowering of insulin receptor expression levels, which leads to impaired glucose clearance following a glucose load^{10,11}. This model raises the possibility that in the absence of such compensatory effects, acute inhibition of IDE may lead to improved physiological glucose tolerance.

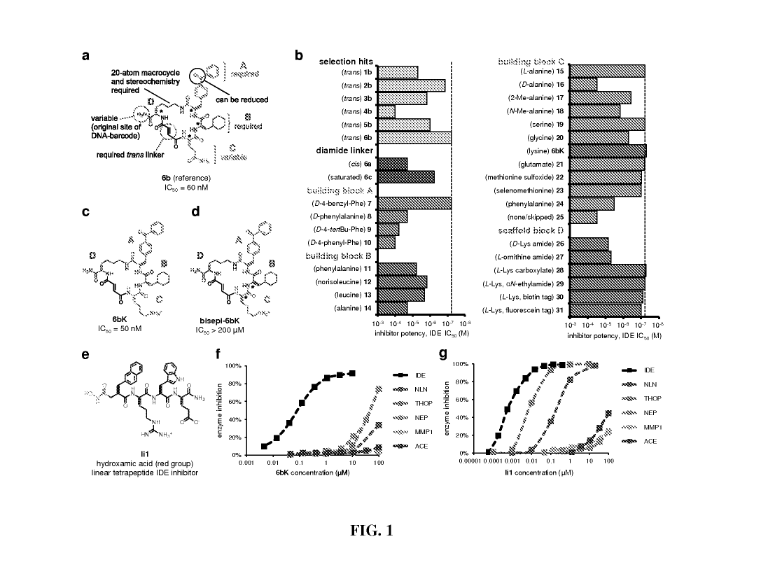

Acute inhibition of enzymes is typically achieved through the use of small-molecule inhibitors, but the only previously reported potent IDE inhibitor, a linear peptide mimetic (Ii1, FIG. 1), is not stable in vivo^{11,12}. These observations highlight the need for a selective small-molecule IDE inhibitor to characterize the biological functions and therapeutic relevance of this enzyme in vivo^{13,14}, uncoupled from confounding physiological adaptations that arise in IDE knock-out mice^{10,11}. In this study we set out to discover potent and selective small-molecule IDE inhibitors that are active in vivo, and to use these compounds to reveal the physiological consequences of IDE inhibition.

### Potent and Selective IDE Inhibitors

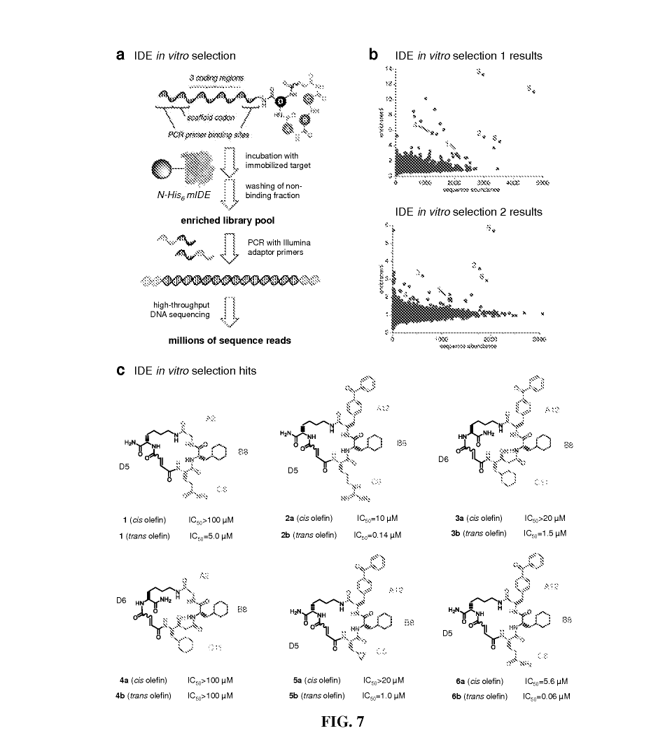

To discover small-molecule modulators of IDE, we performed in vitro selections on a previously described DNA-templated library of 13,824 synthetic macrocycles^{15,16} for the ability to bind immobilized mouse IDE (FIG. 7). Each macrocycle in this library is attached to a unique DNA oligonucleotide that can be used to identify the structures of IDE-binding macrocycles^{16,17}. High-throughput DNA sequencing revealed the macrocycle-encoding DNA templates enriched in two independent IDE selection experiments, resulting in the identification of six putative IDE-binding macrocycles that share common structural features (FIG. 1a, FIG. 7).

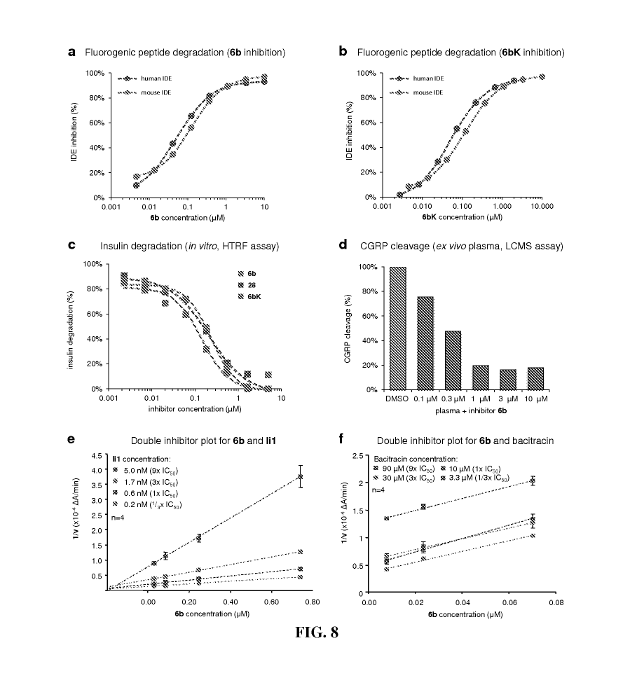

We synthesized these six macrocycles without the attached DNA using solid- and solution-phase synthesis as either of two possible cis- or trans-alkene stereoisomers^{16,17} (FIG. 7). Biochemical assays revealed that four of the six trans-macrocycles assayed were inhibitors of IDE with IC₅₀≦1.5 μM (FIG. 7). The most active inhibitor among the library members enriched in the selection, 20-membered macrocycle 6b (FIG. 1a), potently inhibited human IDE (IC₅₀=60 nM). The ability of 6b to inhibit IDE proteolytic activity in vitro was confirmed by three complementary assays using a fluorogenic peptide, an insulin immunoassay, and a calcitonin-gene related peptide LC-MS assay (Data FIG. 8)¹⁸.

We synthesized and biochemically assayed 30 analogs of 6b in which each building block was systematically varied to elucidate structure-activity relationships of these inhibitors (FIG. 1b). These studies revealed the structural requirements required for potent IDE inhibition by this new class of molecules, including a trans-fused 20-membered macrocycle, the stereochemistry of the macrocycle substituents, and the size, shape, and hydrophobicity of the A and B building blocks (FIG. 1a). In contrast to the strict requirements at positions A and B, different building blocks were tolerated at position C (FIG. 1b). Based on these results, we identified the inhibitor 6bK (IC₅₀=50 nM, FIG. 1c) as an ideal candidate for in vivo studies because it exhibits enhanced water solubility relative to 6b, and can be readily synthesized on gram scale (see Methods section)¹⁴.

Because selectivity is a crucial feature of effective probes to elucidate physiological functions¹⁴, we next characterized the protease inhibition selectivity of 6bK. This inhibitor exhibited ≧1,000-fold selectivity in vitro for IDE over all other metalloproteases tested: thimet oligopeptidase (THOP), neurolysin (NLN), neprilysin, matrix metalloprotease 1, and angiotensin converting-enzyme (FIG. 1f). In contrast, the previously reported substrate mimetic hydroxamic acid inhibitor Ii1¹² (FIG. 1e) is not as selective and it potently inhibits IDE (IC₅₀=0.6 nM), THOP (IC₅₀=6 nM), and NLN (IC₅₀=185 nM) (FIG. 1g). The remarkable selectivity of 6bK, in contrast with the known promiscuity of some active site-directed metalloprotease inhibitors¹⁹, led us to speculate that the macrocycle engages a binding site distinct from the enzyme's active site. This hypothesis was supported by double-inhibitor kinetic assays that revealed synergistic, rather than competitive, inhibition by macrocycle 6b band substrate mimetic Ii1 (FIG. 8).

### Structural Basis of IDE Inhibition

To determine the molecular basis of IDE inhibition and selectivity by these macrocycles, we determined the X-ray crystal structure of catalytically inactive cysteine-free human IDE-E111Q²⁰ bound to 6b at 2.7 Å resolution (FIG. 2, Table 1). The enzyme adopted a closed conformation and its structure is essentially identical to that of apo-IDE (PDB 3QZ2, RMSD=0.257 Å). Macrocycle 6b occupies a binding pocket at the interface of IDE domains 1 and 2 (FIG. 2a), and is positioned more than 11 Å away from the zinc ion in the active site (FIGS. 2b and 2c). This distal binding site is a unique structural feature of IDE compared to related metalloproteases²¹, and does not overlap with the binding site of the substrate-mimetic inhibitor Ii1¹². The structure suggests that by engaging this distal site, the macrocycle competes with substrate binding (FIG. 9) and abrogates key interactions that are necessary to unfold peptide substrates for cleavage²²⁻²⁴ (FIG. 2d).

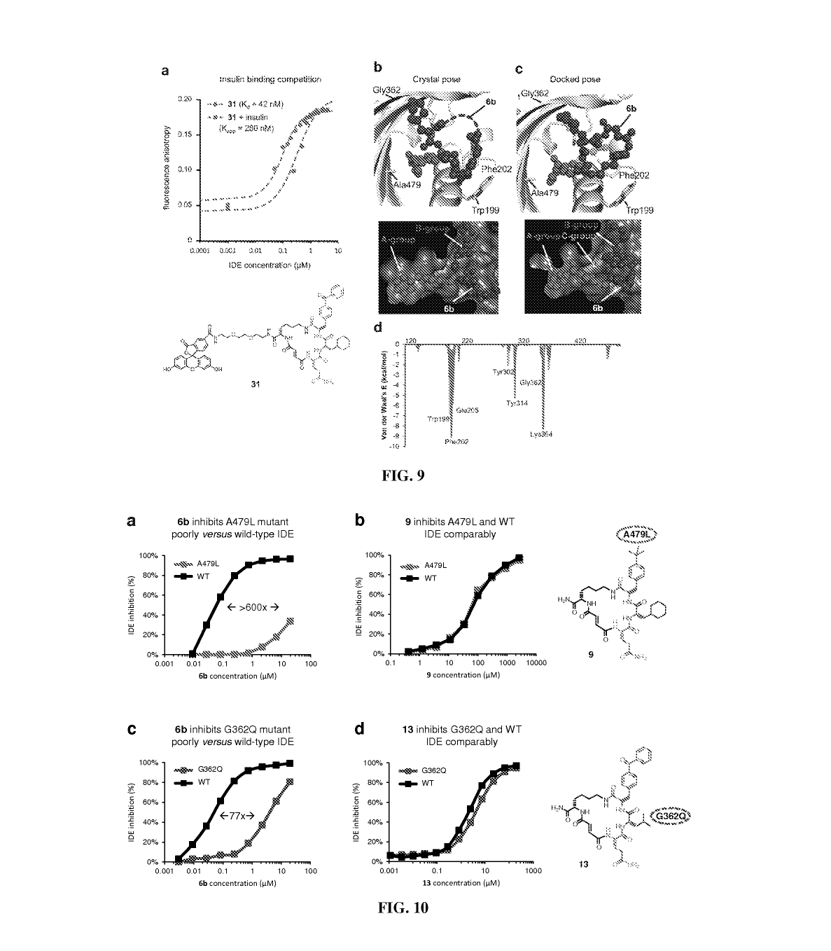

To test the relevance of this structural model of the macrocycle: IDE complex (FIG. 9) in solution, we identified IDE mutations predicted by the structural model to impede 6b binding (FIG. 2e), prepared the corresponding mutant IDE proteins, and measured their abilities to be inhibited by 6b and 6bK. In addition, we also synthesized 6b analogs designed to complement these mutations and rescue inhibitor potency (FIG. 10). Building block A (p-benzoyl-phenylalanine in 6b) occupies a 10 Å-deep pocket in the crystal structure (FIG. 2b), defined by residues Leu201, Gly205, Tyr302, Thr316, and Ala479. As predicted by the structural model, mutation of Ala479 to leucine decreased the potency of inhibitors 6b and 6bK more than 600-fold, consistent with a significant steric clash in the binding site between Leu479 and the distal benzoyl group in building block A (FIG. 2e). Replacement of the p-benzoyl-phenylalanine building block with the smaller tert-butyl-phenylalanine, macrocycle 9, inhibited A479L-IDE with equal potency as wild-type IDE, consistent with the ability of the smaller macrocycle 9 to accommodate the added bulk of the leucine side chain (FIG. 10). Likewise, building block B (cyclohexylalanine in 6b) makes contacts in the structure with the peptide backbone of residues Val360, Gly361, and Gly362 located on the lateral β-strand 13 of IDE domain 2. These residues are thought to assist in unfolding of large peptide substrates by promoting cross-β-sheet interactions²²⁻²⁴. Mutation of Gly362 to glutamine decreased the inhibition potencies of 6b and 6bK at least 50-fold compared to wild-type IDE (FIG. 2e). A modified macrocycle (13) in which the cyclohexylalanine building block was replaced with a smaller leucine residue inhibited G362Q IDE and wild-type IDE comparably, consistent with a model in which the smaller B building block complemented the larger size of the glutamine side chain (FIG. 10). Together, these structural and biochemical studies provide strong evidence for the proposed distal binding site of 6b and demonstrate the ability of the DNA-templated macrocycle library to provide inhibitors that achieve unusual selectivity by targeting residues beyond the catalytic site.

### Inhibition of IDE in vivo

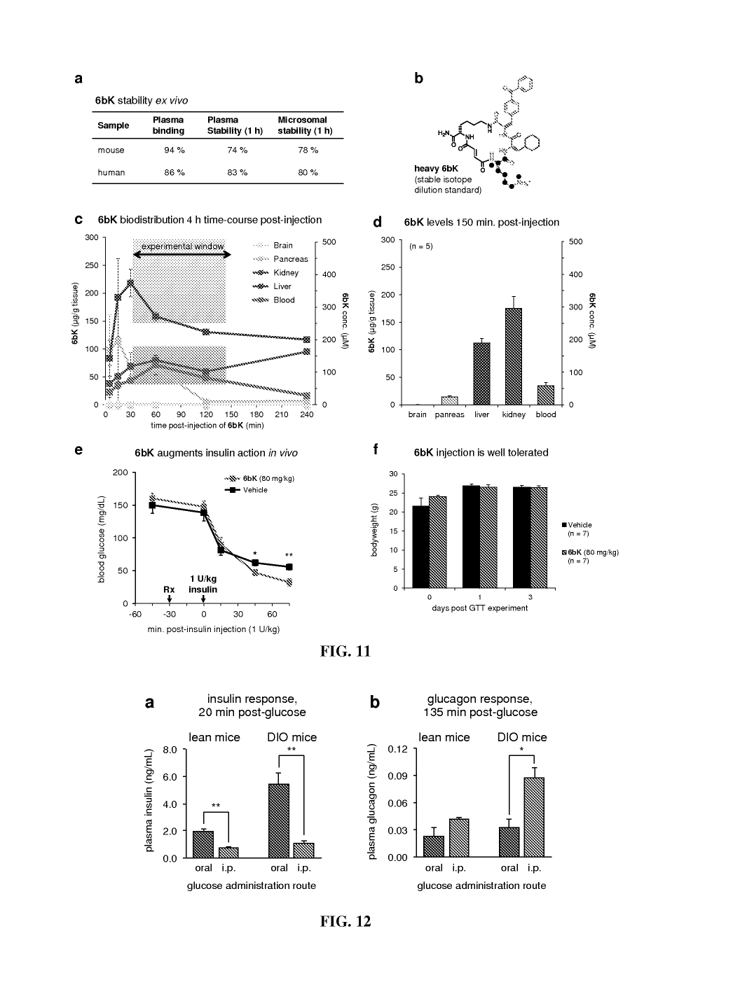

Next we characterized the stability, physicochemical, and pharmacokinetic properties of 6bK. This macrocycle was resistant to hydrolysis during incubations with plasma and liver microsome preparations (74% and 78% remaining after 1 h, respectively), suggesting that this compound may be sufficiently stable in vivo to inhibit IDE activity. Plasma protein binding assays indicated that ˜6% of 6bK remains unbound and potentially available to engage its target. To measure plasma half-life in vivo, we treated mice by intraperitoneal (i.p.) injection of 80 mg/kg 6bK using a formulation of sterile saline and Captisol, a β-cyclodextrin-based agent used to improve solubilization and delivery²⁵. Plasma and tissue concentrations of 6bK were measured using isotope dilution mass spectrometry (IDMS) (FIG. 11). Plasma levels of 6bK were detectable 5 min post-injection, reached peak concentration (>100 μM) at 60 min, and were maintained at a detectable level for at least 4 h (FIG. 11). This circulation time is within the timescale for standard physiological experiments with live animals^{26,27}. We observed prompt biodistribution of 6bK into plasma, liver, kidney, and pancreatic tissues (FIG. 11). We did not detect any6bK in the brain, suggesting that this macrocycle may not inhibit IDE within brain tissues, where IDE activity is needed for the clearance of the β-amyloid peptide¹⁰. Collectively, these observations suggested the viability of 6bK as a potential in vivo IDE inhibitor probe in peripheral tissues of mice¹⁴.

To evaluate the ability of 6bK to inhibit IDE activity in vivo, we subjected non-fasted mice to insulin tolerance tests (ITT)²⁷ following a single injection with 6bK (80 mg/kg) formulated in Captisol²⁵. The insulin injections were carried out 30 min post-injection, the time of highest 6bK concentration in plasma (approximately 100 μM, ˜1000-fold the IC₅₀ for mouse IDE). Following a subcutaneous insulin injection, mice treated with 6bK experienced lower hypoglycemia and higher insulin levels compared to vehicle controls (p<0.01, FIG. 11; see also FIG. 4b). In 1955, Mirsky used a similar ITT assay to suggest the feasibility of insulin stabilization by injecting rats with preparations of an undefined endogenous IDE inhibitor crudely fractionated from bovine livers (presumably a competitive substrate; see Table 2)²⁸.

Our experiments with 6bKprovide the first evidence that a well-defined, selective, and physiologically stable pharmacological IDE inhibitor can augment the abundance and activity of insulin in vivo. Testing the macrocycle at several doses established an effective dose of 6bK of ˜2 mg/mouse i.p., representing 80 mg/kg for lean C57BL/6J mice (25 g), and 60 mg/kg for diet-induced obese (DIO) mice (35-45 g). These inhibitor doses were well tolerated, did not produce adverse reactions, or body weight loss (FIG. 11), and did not induce detectable behavioral abnormalities.

### Anti-Diabetic Activity of IDE Inhibition during Oral Glucose Administration

To determine the physiological consequences of acute IDE inhibition in vivo, we evaluated glucose tolerance of mice treated with 6bK. We used two methods of glucose delivery, either oral gavage or i.p. injection,²⁶ and two different mouse models, lean or DIO mice^{29,30}. These four conditions were chosen to survey the role of IDE activity under a broad range of endogenous insulin levels and insulin sensitivity^{26,29}. Oral glucose administration, for example, results in greater insulin secretion compared to injected glucose delivery (FIG. 12). Passage of glucose through the gut causes the release of GLP-1, which strongly augments glucose-dependent insulin secretion^{2,29}. This phenomenon is referred to as the ‘incretin effect’ (FIG. 12) and is magnified in DIO mice²⁹. In addition, DIO mice display hyperinsulinemia and insulin resistance compared to lean mice, enabling us to test the consequences of IDE inhibition in a model that resembles early type-2 diabetes in humans³⁰.

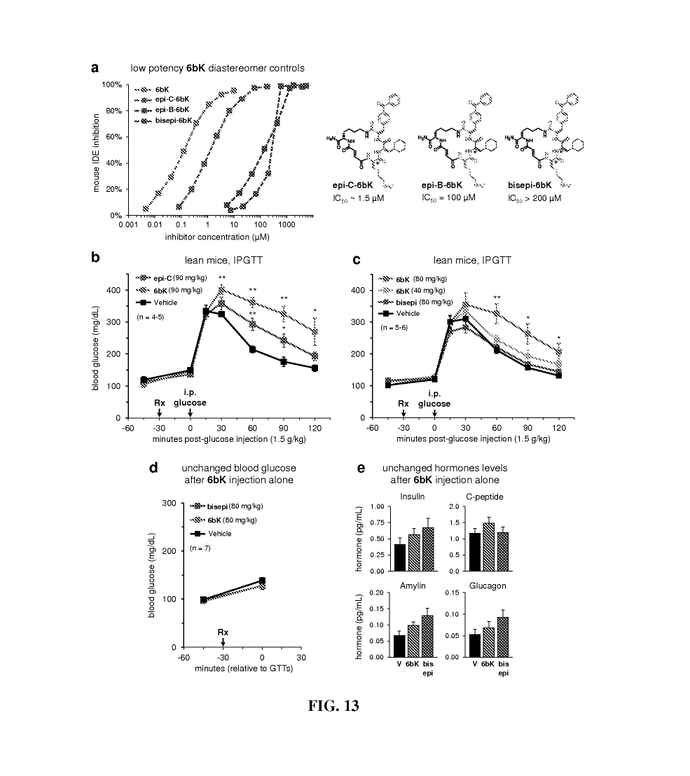

In all glucose tolerance experiments we included two control groups: vehicle alone, and the inactive isomer¹⁴ bisepi-6bK (FIG. 1c), which is identical to 6bK in chemical composition and bond connectivity, but has virtually no IDE inhibition activity (IC₅₀>100 μM, FIG. 1c, see also Data FIG. 13). As expected, administration of 6bK alone, without a glucose challenge, did not significantly alter basal blood glucose or hormone levels compared to control treatments (FIG. 13). We then examined the effect of 6bK on blood glucose levels during an oral glucose tolerance test (OGTT). Lean or DIO mice were fasted overnight for these experiments, and then treated with a single dose of 6bK, vehicle alone, or a matching dose of inactive bisepi-6bK. After 30 minutes, glucose was administered by oral gavage.

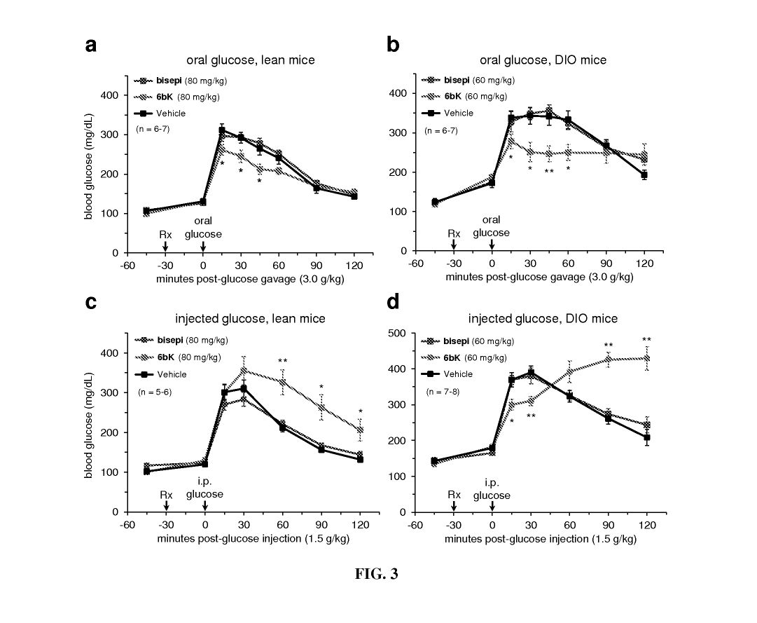

Importantly, both lean and DIO mice treated with 6bK displayed significantly improved oral glucose tolerance compared to vehicle or inactive bisepi-6bK control groups (FIG. 3 and FIG. 13). Effects of similar magnitude on oral glucose tolerance in mice have been observed using several known human anti-diabetic therapeutics³¹⁻³⁴. The two control groups exhibited similar blood glucose profiles, indicating that the observed effects of 6bK on glucose tolerance are lost when the stereochemistry of 6bK is altered in a way that abolishes IDE inhibition.

These observations support a model in which IDE regulates glucose-induced insulin signaling, and therefore glucose tolerance, and demonstrate that acute IDE inhibition improves post-prandial glucose control in lean and DIO mice (FIGS. 3a and 3b). Together, these results represent the first time that IDE inhibition has been shown to improve blood glucose tolerance^{3,28}.

### IDE Inhibition During an Injected Glucose Challenge Leads to Impaired Glucose Tolerance

Prior work using IDE^{-/-} mice characterized the effect of i.p. glucose injections, therefore we repeated the above experiments with 6bK followed by i.p. injected glucose tolerance tests (IPGTTs) to provide a more direct comparison with the knockout animal experiments^{10,11}. In contrast to the observed improvement in oral glucose tolerance upon 6bK treatment (FIGS. 3a and 3b), IDE inhibition with 6bK followed by a glucose injection (1.5 g/kg i.p.) resulted in impaired glucose tolerance after 2 h in both lean and obese mice compared to vehicle alone or bisepi-6bK-treated controls (FIGS. 3c and 3d). These changes in the glucose response profiles of lean mice treated with 6bK compared to vehicle controls resemble the reported differences between IDE^{-/-} and IDE^{+/+} mice during similar IPGTTs^{10,11}. Moreover, DIO mice treated with 6bK followed by glucose injection displayed a biphasic response in which glucose levels are lower over the initial 30 minutes of the IPGTT, followed by a hyperglycemic “rebound” starting 1 h after glucose injection (FIG. 3d). Both the suppression of peak glucose levels and the magnitude of the hyperglycemic rebound were dependent on 6bK dose, and neither effect was observed in cohorts treated with vehicle alone or inactive bisepi-6bK, indicating that the impaired glucose tolerance during an IPGTT correlates with IDE activity (FIG. 3, and FIG. 13).

Collectively, the results of the OGTTs and IPGTTs indicate that the route of glucose administration impacts the physiological response of 6bK-treated animals in ways that cannot be explained by a simple model in which IDE's physiological role is only to degrade insulin. Instead, the IPGTT results strongly suggest a role for IDE in regulating other glucose-regulating peptide hormones in vivo (Table 2).

IDE Regulates Multiple Hormones in vivo

The biochemical properties of IDE and its substrate recognition mechanism²¹⁻²³ enable this enzyme to cleave a wide range of peptide substrates in vitro (Table 2). Two glucose-regulating hormones, beyond insulin, that are potential candidates for physiological regulation by IDE during a glucose challenge are glucagon and amylin. Purified IDE has been previously shown to cleave both peptides in vitro³⁵⁻³⁷, but neither hormone is known to be regulated by IDE activity in vivo. Compared to insulin, glucagon is a modest in vitro IDE substrate (K_{M}=3.5 μM for glucagon versus K_{M}<30 nM for insulin)³⁵, although IDE is capable of degrading glucagon at a comparable rate if present in sufficiently high concentrations (k_{cat}=38 min⁻¹ for glucagon)³⁶. Amylin is also a substrate for IDE in vitro (K_{M}=˜0.3 μM)³⁷. Other proteases suggested to degrade glucagon include nardilysin, cathepsin B and D, in cells and in vitro^{38,39}, and neprilysin, which was shown to play a role in renal clearance of glucagon⁴⁰. However, none of these enzymes are known to regulate endogenous processing of hormones or modulate blood glucose levels. To our knowledge, no proteases have been previously shown to degrade amylin in vivo³⁷.

To begin to probe the possibility that glucagon or amylin is regulated in vivo by IDE, we measured the plasma levels of these hormones at 20 and 130 minutes post-glucose injection in DIO mice treated with 6bK or vehicle alone during an IPGTT (FIG. 4a). Plasma collected 20 minutes post-glucose injection showed elevated insulin and amylin levels, but unchanged glucagon levels, for the 6bK-treated cohort relative to the control group (FIG. 4a). During the hyperglycemic rebound 130 min post-injection, glucagon levels for the 6bK group were strongly elevated above 200 pg/mL, compared with 90 pg/mL glucagon in control mice (FIG. 4a). Consistent with these elevated glucagon levels, expression of a gluconeogenesis transcriptional marker, G6Pase, was elevated in the livers of 6bK-treated mice compared to control mice (FIG. 4a).

Because hormone abundance measurements can be difficult to interpret during fluctuations in blood glucose that in turn affect pancreatic hormone secretion, we performed additional studies to confirm the relationship between IDE activity and glucagon and amylin levels in vivo. To more directly establish the effect of IDE inhibition on the clearance of insulin, amylin, and glucagon in vivo, we injected each of these three hormones into lean mice 30 min after treatment with 6bK or vehicle alone (FIGS. 4b to 4d). The 6bK-treated cohorts exhibited significantly stronger blood glucose responses to each of these hormones compared to vehicle controls; mice treated with 6bK experienced hypoglycemia during insulin tolerance tests (FIG. 4b) and hyperglycemia following challenges with either amylin (FIG. 4c) or glucagon (FIG. 4d) relative to control animals. Similar to glucagon, acute amylin administration in rodents results in a transient increase in blood glucose levels through gluconeogenesis and activation of lactic acid flux from muscle tissue to the liver⁴¹. Moreover, in each case the plasma level of the hormone injected remained elevated at the end of the procedure in 6bK-treated mice relative to control animals, demonstrating a role for IDE in regulating the abundance of these hormones (FIGS. 4b to 4d insets).

### IDE Inhibition Promotes Glucagon Signaling and Gluconeogenesis

Taken together, these results strongly suggest that IDE activity regulates the stability and physiological activities of glucagon and amylin, in addition to insulin. Higher glucagon levels upon 6bK treatment provide a possible explanation for impaired glucose tolerance observed during an IPGTT. This model predicts that abrogating glucagon signaling should reverse the elevation of blood glucose by 6bK treatment during an IPGTT, while not substantially affecting the signaling pathways through which 6bK treatment lowers blood glucose during an OGTT.

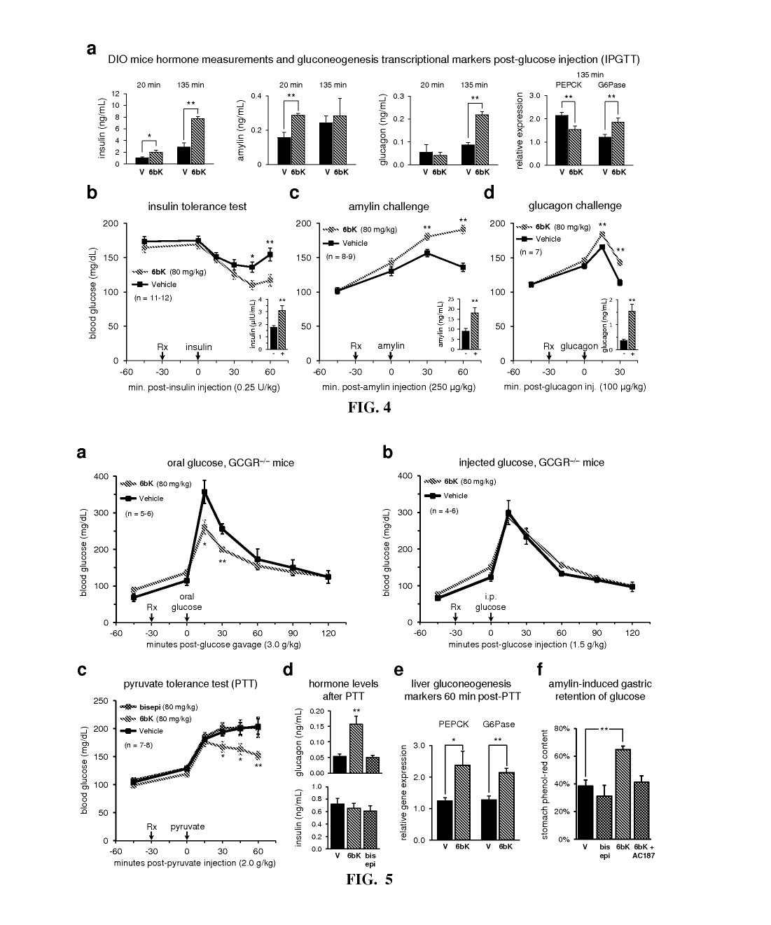

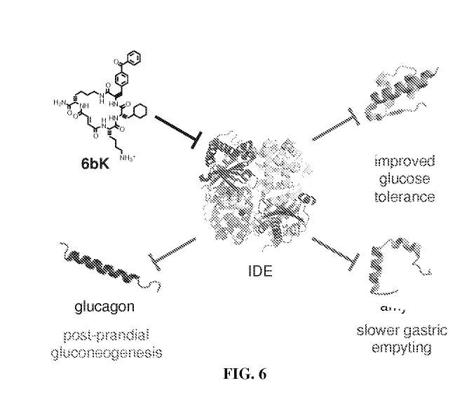

To explicitly test these hypotheses, we repeated the glucose tolerance experiments using GCGR^{-/-} mice that lack the G-protein coupled glucagon receptor (FIGS. 5a and 5b)⁴². As expected, mice lacking glucagon signaling exhibited an improvement in oral glucose tolerance upon 6bK treatment relative to vehicle controls that was similar to the oral glucose tolerance improvement observed in wild-type mice (FIG. 5a), consistent with a model in which insulin signaling in these mice is intact and regulated by IDE. In contrast, the responses of GCGR^{-/-} mice treated with 6bK or vehicle alone to i.p. injected glucose were virtually identical, consistent with a model in which glucagon signaling drives the impaired glucose tolerance of wild-type lean and DIO mice upon 6bK treatment during an IPGTT (compare FIGS. 5b and 3c). Collectively, these results reveal that IDE inhibition promotes glucagon signaling that can impair glucose tolerance during an IPGTT.

To investigate the effect of IDE inhibition on endogenously secreted glucagon, we subjected mice treated with 6bK, vehicle, or inactive bisepi-6bK to a pyruvate tolerance test (PTT), which measures the ability of the liver under the action of glucagon to use pyruvate as a gluconeogenic substrate (FIG. 5c). The 6bK-treated cohort displayed significantly elevated plasma glucagon and increased expression of liver gluconeogenic markers compared to both control groups (FIGS. 5d and 5e). The 6bK cohort also experienced significantly lower blood glucose during the PTT, suggesting that IDE inhibition produced a concomitant stimulation of glucose clearance that outweighed its effects on gluconeogenesis (FIG. 5c). These results collectively establish that IDE inhibition can augment endogenous glucagon signaling under conditions that favor gluconeogenesis.

### IDE Inhibition Promotes Amylin Signaling and Gastric Emptying

Amylin is co-secreted with insulin, and is involved in glycemic control by inhibiting gastric emptying through the vagal route⁴³, promoting satiety during meals⁴⁴, and antagonizing glucagon signaling⁴⁵. Pramlintide (Smylin) is a synthetic amylin derivative used clinically to control post-prandial glucose levels^{31,34,46}. To determine the effects of IDE inhibition on endogenous amylin signaling, we measured gastric emptying efficiency⁴⁷, an amylin-specific process, in mice treated with 6bK, inactive control bisepi-6bK, or vehicle alone. Mice treated with 6bK exhibited 2-fold slower gastric emptying of a labeled glucose solution measured at 30 minutes post-gavage compared to vehicle and bisepi-6K-treated controls (FIG. 5f). Importantly, co-administration of the specific amylin receptor antagonist AC187⁴⁸ blocked the effects of 6bxzK on gastric emptying (FIG. 5f). Collectively, these data reveal that IDE inhibition can slow post-prandial gastric emptying and demonstrate a role for IDE in modulating amylin signaling in vivo. These results also suggest that IDE inhibition may mimic the effect of amylin supplementation with pramlintide during meals^{2,31}.

### Discussion

The discovery and application of the first potent, highly selective, and physiologically active small-molecule IDE inhibitor revealed that acute IDE inhibition can lead to improved glucose tolerance in lean and obese mice after oral glucose administration, conditions that mimic the intake of a meal. These results validate the potential of IDE as a therapeutic target following decades of speculation^{3,4}. Our data show that small-molecule IDE inhibitors can improve oral glucose tolerance to an extent comparable to that of DPP4 inhibitors^{2,31}. The potential relevance of these animal studies to human disease is supported by the repeated recognition of IDE as a diabetes susceptibility gene in humans⁵⁻⁹.

Equally important, additional in vivo and biochemical experiments using 6bK led to the discovery that IDE regulates the stability and signaling of glucagon and amylin, in addition to its established role in insulin degradation¹⁰⁻¹². The identification of two additional pancreatic hormones as endogenous IDE substrates advances our understanding of the role of IDE in regulating physiological glucose homeostasis (FIG. 6). Amylin-mediated effects on gastric emptying and satiety during meals have been recently recognized to have therapeutic relevance in the treatment of diabetes^{2,31}, and our results represent the first demonstration of a small molecule that can regulate amylin signaling. Moreover, the link between IDE and glucagon provides additional evidence of the importance of glucagon regulation in human diabetes⁴⁹.

This example also reveals a specific pharmacological requirement for therapeutic IDE inhibition—namely, that transient, rather than chronic, IDE inhibition is desirable to prevent elevation of glucagon signaling⁴⁹ (FIG. 6). A potential anti-diabetic therapeutic strategy motivated by these findings is the development of a fast-acting IDE inhibitor that can be taken with a meal to transiently augment endogenous insulin and amylin responses to help control post-prandial glycemia³¹⁻³², and that is cleared or degraded before glucagon secretion resumes. Similar pre-meal therapeutic strategies with short-lived agents have already proven successful with fast-acting insulin analogs, secretagogues, and amylin supplementation³²⁻³³. It is tempting to speculate these agents may also have synergistic effects when co-administered with an IDE inhibitor^{3,50}. Alternatively, the combination of an IDE inhibitor with incretin therapy^{2,31} or a glucagon receptor antagonist⁴⁹, may also prove therapeutic, as suggested by our experiments with glucagon-receptor deficient mice.

The unbiased in vitro selection approach used to discover 6bK and the IDE-macrocycle co-crystal structure revealed a novel small molecule-binding site that enables unprecedented inhibition selectivity for this enzyme. This structural insight and these macrocycles has been translated into the assays described herein that use competitive probes specifically targeting this binding site for the discovery of novel therapeutic leads that target IDE inhibition.

### Materials and Methods

All data points and error bars represent mean±SEM. Significance tests were performed using two-tail Student's t-test, and significance levels shown in the figures are p<0.05 (*) or p<0.01 (**) versus the vehicle-only control group. The in vitro selection of the DNA-templated macrocycle libraryl^{16,17} used 20 μg His₆-tagged mouse IDE immobilized on cobalt magnetic beads (Dynabeads, Invitrogen). IDE proteolytic activity was assayed with fluorogenic peptide Mca-RPPGFSAFK(Dnp)-OH (R&D). IDE inhibition was confirmed using an anti-insulin antibody time-resolved FRET assay (Cysbio), and using an LCMS assay for CGRP cleavage fragments CGRP₁₋₁₇ and CGRP₁₈₋₃₇ in plasma¹⁸. Macrocyclic inhibitors were synthesized by Fmoc-based solid-phase synthesis using Rink amide resin (NovaPEG, Novabiochem), and purified by HPLC. Inhibitor Ii1 was synthesized as previously reported¹² and purified by HPLC. Stable-isotope LCMS quantitation of 6bK in biological samples was performed by spiking heavy-labeled 6bK synthesized using ¹³C₆¹⁵N₂ lysine (Sigma-Aldrich).

Wild-type lean C57BL/6J and diet-induced obese (DIO) C57BL/6J age-matched male mice were purchased from Jackson Laboratories, and used at ages 14-16, and 24-26 weeks respectively (>20 weeks of high-fat diet for the DIO mice). GCGR^{/} mice were bred from heterozygous mice and used between 11 and 17 weeks. Animals were fasted overnight 14 h for all experiments, except for the insulin tolerance test, which required 5 h of fasting during the morning. Blood glucose was measured from tail nicks using AccuCheck (Aviva) meters. Insulin (Humulin-R, Eli Lilly) and amylin (Bachem) were formulated in sterile saline (5 mL/kg), glucagon (Eli Lilly) was formulated in 0.5% BSA sterile saline (5 mL/kg), and glucose was formulated in sterile saline (3 g in 10 mL total).

Trunk blood was obtained for plasma hormone measurements using the Multiplexed Mouse Metabolic Hormone panel (Milliplex, EMD Millipore) on a Luminex FlexMap 3D instrument. Gastric emptying was measured by dissection of stomachs 30 min after an oral glucose bolus (3.0 g/kg, 10 mL/kg) in sterile saline containing 0.1 mg/mL phenol red.

Total RNA was isolated from liver samples (˜100 mg) using TRIzol (Invitrogen) and purified using an RNeasy® kit (Qiagen) and on-column DNAse treatment (Qiagen). Reverse transcription was performed with oligo(dT) primers using SuperScript III (Life Technologies). RT-PCR was performed with two primer pairs per target normalized against tubulin and beta-actin transcripts (ΔΔC_{T} method).

In vitro selection of a DNA-templated library with immobilized mouse IDE. The in vitro selection used here was adapted from previously described protocols⁵¹⁻⁵² using a DNA-templated library of 13,824 macrocycles⁵³. Recombinant N-His₆-tagged mouse IDE₄₂₋₁₀₁₉ (isoform containing the amino acids 42-1019 of the IDE sequence) was purified using immobilized cobalt magnetic beads (Dynabeads® His-Tag Isolation & Pulldown, Invitrogen®) according to the manufacturer's instructions. This purified IDE was confirmed to be catalytically active using the peptide substrate assay described below. IDE protein (˜20 μg) was loaded onto the solid support by incubating the protein with beads (30 μL) at 25° C. for 30 min in 300 μL of pH 8.0 buffer containing 50 mM phosphate, 300 mM NaC1 and 0.01% Tween-20 (PBST buffer), and washed twice with the same buffer. Two individually prepared protein-bead suspensions were incubated for 30 min with 5 pmol of the DNA-templated macrocycle library at RT, in pH 7.4 buffer containing 50 mM Tris-HCl, 150 mM NaCl, 0.05% Tween-20 (TBST buffer) supplemented with 0.01% BSA and 3 mg/mL yeast RNA (Ambion®). The beads were washed three times with 200 μL TBST buffer. The enriched library fraction was eluted by treatment with 200 mM imidazole in PBST buffer (50 μL) for 5 min.

The eluate solution was isolated and purified by buffer exchange using Sephadex spin-columns (Centrisep, Princeton Separation), according to the manufacturer's instructions. PCR amplification of the enriched pool of library barcodes was performed as previously reported⁵¹, using primers that append adaptors for Illumina sequencing and a 7-base identifier. The long adaptor primer was 5′-AATGATACGGCGACCACCGAGATCTACACTCTTTCCCTACACGACGC TCTTCCGATCTXXXXXXCCCTGTACAC and the short adaptor primer was 5′-CAAGCAG AAGACGGCATACGAGCTCTTCCGATCTGAGTGGGATG (the 7-base identifier was XXXXXX). The PCR amplicons were purified by polyacrylamide gel electrophoresis, extracted, and quantified using qPCR and Picogreen assays (Invitrogen).

High-throughput DNA sequencing was performed on an Illumina Genome Analyzer instrument at the Harvard FAS Center for Systems Biology, Cambridge, Mass., to yield an average of ˜3.8 million sequence reads for each selection, untreated bead control and pre-selection library. Deconvolution of library barcodes and enrichment calculations were performed with custom software as described previously⁵¹. Variations in library member abundance as a result of binding to immobilized IDE was revealed by calculating fold-enrichment over the pre-selection library for the two independent selection experiments (FIG. 7).

Protease assays with fluorogenic peptide substrates. The proteases IDE₄₂₋₁₀₁₉, recombinant human IDE₄₂₋₁₀₁₉ (R&D Systems), neprilysin (R&D), and angiotensin-converting enzyme (R&D) were assayed using the fluorophore/quencher-tagged peptide substrate Mca-RPPGFSAFK(Dnp)-OH (R&D) according to the manufacturer's instructions and recommended buffers. For IDE the recommended buffer is 50 mM Tris pH 7.5, 1 M NaCl. The enzyme mixtures (48 μL) were transferred to a 96-well plate and combined with 2 μL of inhibitor in DMSO solutions, in 3-fold dilution series. The mixtures were allowed to equilibrate for 10 min and the enzymatic reaction was started by addition of substrate peptide in assay buffer (50 μL), mixed, and monitored on a fluorescence plate reader (excitation at 320 nm, emission at 405 nm). Similarly, thimet oligopeptidase (R&D) and neurolysin (R&D) were assayed using substrate Mca-PLGPK(Dnp)-OH (R&D) according to the manufacturer's instructions and recommended buffers. Matrix metalloproteinase-1 (R&D) was activated and assayed according to the manufacturer's instructions with substrate Mca-KPLGL-Dpa-AR-NH₂ (R&D). All assay data points were obtained in duplicate. Data for the Yonetani-Theorell double inhibition plot was generated using 1 μL of each inhibitor (6bK, and Ii1 or bacitracin) under otherwise identical conditions as above, at concentrations corresponding to ⅓×, 1×, 3× and 9× of respective IC₅₀ values against hIDE (R&D).^{54,55}

IDE degradation assays for insulin and calcitonin-gene related peptide (CGRP). A solution of 0.4 μg/mL IDE (R&D) in pH 7.5 buffer containing 50 mM Tris, 1.0 M NaCl (48 μL) was transferred to a 96-well plate, and combined with 2 μL of each inhibitor (6b, 6bK and 28) dissolved in DMSO, in 3-fold dilution series (FIG. 8c). A solution of insulin (50 μL) was added to a final concentration of 10 ng/mL, and incubated at 30° C. for 15 min. This procedure was optimized to result in ˜75% degradation of insulin. The reaction was terminated by addition of inhibitor 6bK to a final concentration of 20 μM and chilled on ice. Insulin was quantified using 10 μL of the enzymatic reaction for the sensitive-range protocol Homogeneous Time-Resolved FRET Insulin assay (CysBio®) in 20 μL total volume according to the manufacturer's instructions. Fluorescence was measured using a Tecan Safire 2 plate reader (excitation=320 nm, emission=665 and 620 nm, lag time=60 μs) according the assay manufacturer's recommendations. Degradation of CGRP by endogenous IDE in mouse plasma was analyzed by LC-MS for the formation of CGRP₁₋₁₇ and CGRP₁₈₋₃₇ as previously reported⁶. The substrate was added to a final concentration of 10 μM, and 6b was added to a final concentration of 0.1 to 10 μM (FIG. 8d).

General Procedure for Synthesis of Macrocycle Inhibitors.

Rink amide resin (NovaPEG Novabiochem®, ˜0.49 mmol/g, typically at a scale of 0.1 to 2 mmol) was swollen with ˜10 volumes of anhydrous DMF for 1 h in a peptide synthesis vessel with mixing provided by dry nitrogen bubbling. In a separate flask, N^{α}-allyloxycarbonyl-N^{ε}-2-Fmoc-L-lysine (5 equiv.) and 2-(1H-7-azabenzotriazol-1-yl)-1,1,3,3-tetramethyl uronium hexafluorophosphate (HATU, 4.75 equiv.) were dissolved in anhydrous DMF (˜10 vol.), then treated with N,N′-diisopropylethylamine (DIPEA, 10 equiv.) for 5 min at RT. The solution was combined with the pre-swollen Rink amide resin and mixed with nitrogen bubbling overnight. The vessel was eluted and the resin was washed three times with N-methyl-2-pyrrolidone (NMP, ˜10 vol.). Following each coupling step, Fmoc deprotection was effected with 20% piperidine in NMP (˜20 vol.) for 20 min, repeated three times, followed by washing three times with NMP (˜10 vol.) and twice with anhydrous DMF (˜10 vol.). The general procedure for amide coupling of building blocks A, B and C was treatment of the resin with solutions of HATU-activated N^{α}-Fmoc amino acids (5 equiv.) for 3-5 hours in anhydrous DMF, mixing with dry nitrogen bubbling. The general procedure for HATU-activation was treating a solution of N^{α}-Fmoc amino acid (5 equiv.) and HATU (4.75 equiv.) in anhydrous DMF (10 vol.) with DIPEA (10 equiv.) for 5 min at RT.

Following the final Fmoc deprotection procedure, the α-amine of building block C was coupled with allyl fumarate monoester (10 equiv.) using activation conditions as previously described with HATU (9.5 equiv.) and DIPEA (20 equiv.) in anhydrous DMF (˜10 vol.). Allyl fumarate coupling was accomplished by overnight mixing with dry nitrogen bubbling, followed by washing five times with NMP (˜10 vol.) and three times with CHCl₃ (˜10 vol.). Simultaneous allyl ester and N-allyloxycarbonyl group cleavage in solid support was effected with three consecutive treatments with a solution of tetrakis(triphenylphosphine)palladium(0) (0.5 equiv.) dissolved in degassed CHCl₃ containing acetic acid and N-methylmorpholine (40:2:1 ratio, ˜20 vol.), mixing by nitrogen bubbling for 30 min. The resin was then washed twice subsequently with ˜20 vol. of 5% DIPEA in DMF, then twice with a 5% solution of sodium diethyldithiocarbamate trihydrate in DMF (˜20 vol.), twice with 5% solution of hydroxybenzotriazole monohydrate in DMF, and finally washed with 50% CH₂Cl₂ in DMF and re-equilibrated with anhydrous DMF (˜10 vol.).

Treating the resin with pentafluorophenyl diphenylphosphinate (5 equiv.) and DIPEA (10 equiv.) in anhydrous DMF (˜10 vol.) mixing by nitrogen bubbling overnight produced the macrocyclized products. The resin was washed with NMP (˜20 vol.) and CH₂Cl₂ (˜20 vol.) and dried by vacuum. The macrocyclized product was cleaved from the resin by two 15 min treatments of the macrocycle-bound resin with 95% TFA containing 2.5% water and 2.5% triisopropylsilane (˜20 vol.), followed by two TFA washes (˜5 vol.). The TFA solution was dried to a residue under rotatory evaporation, and the peptide was precipitated by the addition of dry Et₂O. The ether was decanted and the remaining solid was dried and dissolved in a minimum volume of 3:1 DMF-water prior to purification by liquid chromatography. HPLC purification was performed on a C18 21.2×250 mm column (5 μm particle, 100 Å pore size, Kromasil®), using a gradient of 30% to 80% MeCN/water over 30 min, and solvents containing 0.1% TFA. Fractions containing the desired macrocyclic peptide were combined and freeze-dried to produce a white powder. Typical yields were 5-15% based on resin loading. Purity was determined by HPLC (Zorbax SB-C18 2.1×150 mm column, 5 μm particle) with UV detection at 230 nm, using a gradient of 30% to 80% MeCN/water over 30 min, and solvents containing 0.1% TFA. The formula of final products was confirmed by accurate mass measurements using an Agilent 1100 LC-MSD SL instrument (Table 4).

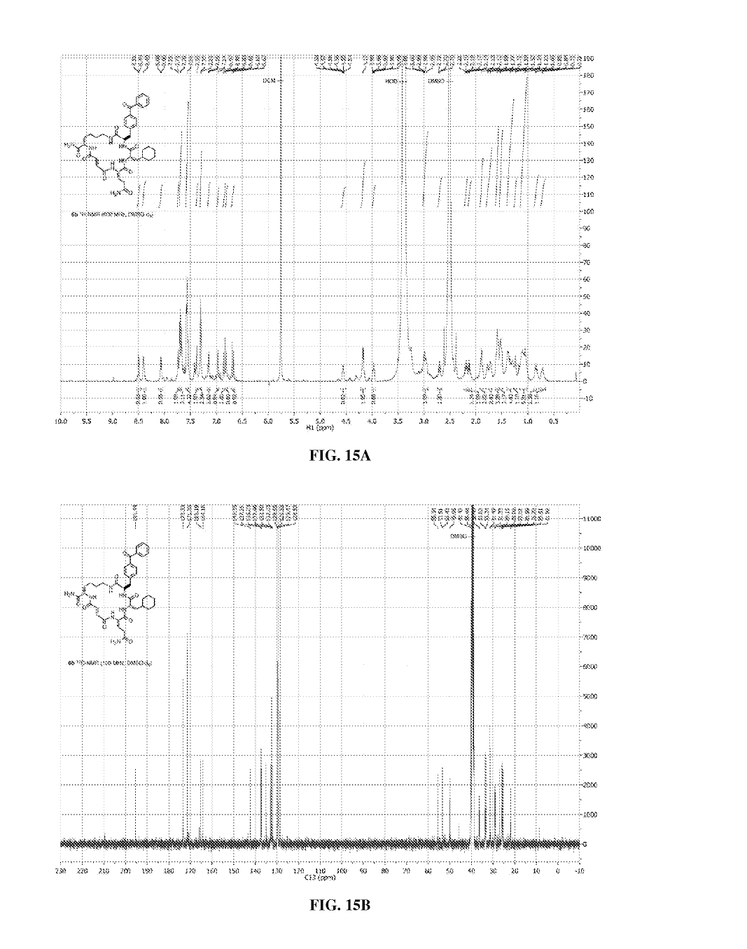

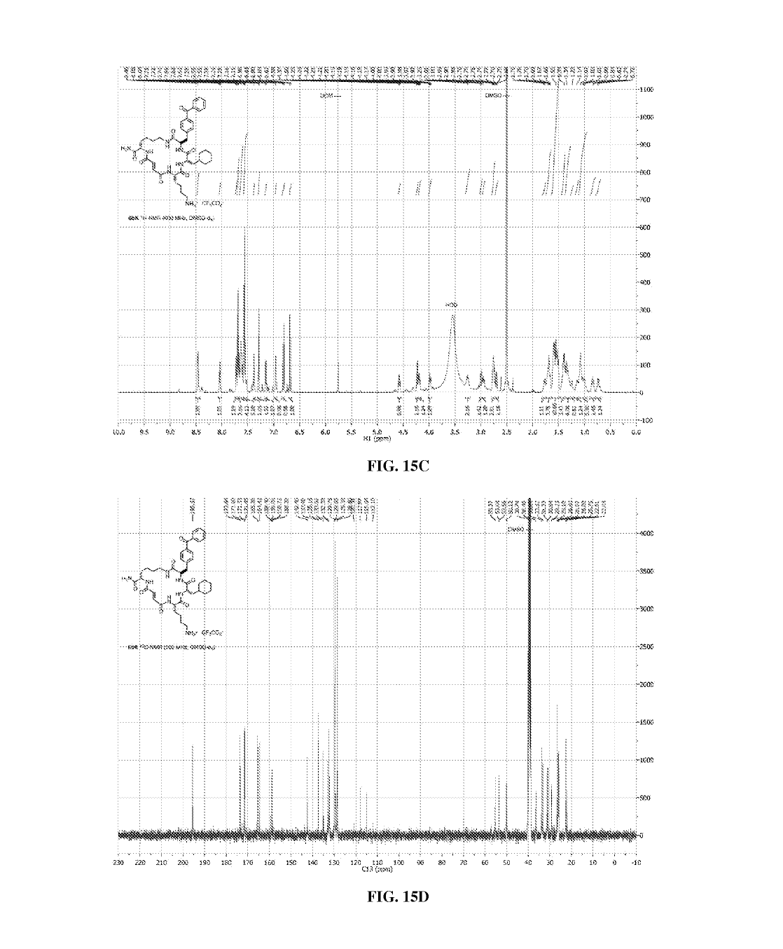

Spectra for Inhibitor 6b (FIGS. 15A and B)

¹H NMR (600 MHz, DMSO-d₆) δ 8.50 (d, J=7.8 Hz, 1H), 8.42-8.36 (m, 1H), 8.07 (d, J=9.0 Hz, 1H), 7.74 (d, J=7.3 Hz, 1H), 7.71-7.65 (m, 3H), 7.59-7.54 (m, 4H), 7.38 (s, 1H), 7.31-7.28 (m, J=8.1 Hz, 2H), 7.15 (d, J=8.6 Hz, 1H), 6.97 (s, 1H), 6.85 (d, J=15.8 Hz, 1H), 6.81 (s, 1H), 6.68 (d, J=15.8 Hz, 1H), 4.56 (td, J=9.3, 4.3 Hz, 1H), 4.21-4.14 (m, J=6.9 Hz, 2H), 3.97 (ddd, J=10.2, 7.5, 2.9 Hz, 1H), 3.03-2.93 (m, 3H), 2.70 (dd, J=13.4, 10.2 Hz, 1H), 2.19 (td, J=15.0, 7.6 Hz, 1H), 2.13 (td, J=15.4, 7.9 Hz, 1H), 1.92-1.86 (m, 2H), 1.82-1.68 (m, 2H), 1.63-1.55 (m, 3H), 1.55-1.48 (m, J=9.0 Hz, 3H), 1.42-1.26 (m, 4H), 1.26-1.22 (m, 1H), 1.15-1.00 (m, 5H), 0.83 (dd, J=21.5, 9.6 Hz, 1H), 0.71 (dd, J=24.8, 13.9 Hz, 1H).

¹³C NMR (100 MHz, DMSO-d₆) δ 195.44, 173.33 (2C), 171.35 (3C), 165.19, 164.16, 142.35, 137.28, 135.03, 132.96, 132.50 (2C), 132.03, 129.65 (2C), 129.53 (2C), 129.47, 128.53 (2C), 55.54, 53.51, 53.43, 49.95, 40.43, 38.98, 36.40, 33.63, 33.24, 31.47, 31.33, 29.15, 29.02, 27.02, 25.99, 25.78, 25.51, 21.92.

High resolution mass, calculated [M+H]⁺=758.3872, found 758.3878, Δ=0.791 ppm.

Spectra for Inhibitor 6bK (Trifluoroacetate Salt) (FIGS. 15C and D)

¹H NMR (600 MHz, DMSO-d₆) δ 8.49-8.44 (m, 2H), 8.04 (d, J=8.8 Hz, 1H), 7.72 (d, J=7.1 Hz, 1H), 7.71-7.67 (m, J=3.1 Hz, 2H), 7.67-7.59 (m, 3H), 7.59-7.52 (m, 4H), 7.38 (s, 1H), 7.29 (d, J=8.2 Hz, 2H), 7.15 (d, J=8.6 Hz, 1H), 6.96 (s, J=2.1 Hz, 1H), 6.75 (dd, J=81.5, 15.8 Hz, 2H), 4.58 (td, J=9.2, 4.2 Hz, 1H), 4.23 (dd, J=15.6, 7.8 Hz, 1H), 4.19 (ddd, J=10.4, 8.4, 3.8 Hz, 1H), 3.98 (ddd, J=11.1, 7.0, 3.0 Hz, 1H), 3.25 (ddd, J=21.3, 13.6, 7.2 Hz, 1H), 2.99 (dt, J=12.6, 7.9 Hz, 1H), 2.95 (ddd, J=13.3, 10.5, 5.2 Hz, 1H), 2.76 (tt, J=12.7, 6.5 Hz, 2H), 2.70 (dd, J=13.3, 10.0 Hz, 1H), 1.81-1.74 (m, 1H), 1.73-1.65 (m, 3H), 1.63-1.48 (m, 8H), 1.45-1.38 (m, 3H), 1.37-1.20 (m, 4H), 1.18-1.11 (m, 1H), 1.11-0.96 (m, 4H), 0.89-0.79 (m, 1H), 0.73 (qd, J=12.6, 3.6 Hz, 1H).

¹³C NMR (100 MHz, DMSO-d₆) δ 195.57, 173.64, 171.60, 171.53, 171.45, 165.36, 164.42, 158.89 (TFA, q, J=34.0 Hz), 142.40, 137.40, 135.16, 133.02, 132.58 (2C), 132.14, 129.75 (2C), 129.65 (2C), 129.58, 128.60 (2C), 116.50 (TFA, q, J=294.8 Hz), 55.37, 53.68, 53.56, 50.12, 38.79, 36.46, 33.84, 33.32, 31.33, 30.94, 29.23, 29.10, 26.60, 26.07, 26.00, 25.75, 22.61, 22.03.

High resolution mass, calculated [M+H]⁺=758.4236, found 758.4233, Δ=−0.396 ppm.

Spectra for bisepi-6bK (Trifluoroacetate Salt) (FIGS. 15E and F)

¹H NMR (600 MHz, DMSO-d₆) δ 8.45 (d, J=6.5 Hz, 1H), 8.38 (d, J=8.9 Hz, 1H), 7.73-7.65 (m, 5H), 7.65-7.59 (m, J=8.2 Hz, 5H), 7.57 (dd, J=14.5, 6.7 Hz, 3H), 7.39 (s, 1H), 7.30 (d, J=8.2 Hz, 2H), 7.01 (s, 1H), 6.90 (s, 2H), 4.32 (ddd, J=14.6, 10.0, 4.0 Hz, 2H), 4.21 (dd, J=13.3, 6.3 Hz, 1H), 3.99 (dt, J=8.3, 6.4 Hz, 1H), 3.11 (dd, J=13.8, 5.9 Hz, 1H), 3.08-3.00 (m, 2H), 2.82 (dd, J=12.7, 5.7 Hz, 1H), 2.79-2.72 (m, 2H), 1.83-1.76 (m, J=7.2 Hz, 1H), 1.73 (d, J=11.3 Hz, 1H), 1.68-1.60 (m, J=9.4 Hz, 4H), 1.55 (qd, J=14.1, 7.5 Hz, 6H), 1.46-1.33 (m, 3H), 1.33-1.20 (m, 4H), 1.10 (d, J=9.2 Hz, 3H), 1.02 (dd, J=23.9, 11.8 Hz, 1H), 0.91 (dd, J=21.9, 10.1 Hz, 1H), 0.79 (dd, J=22.0, 10.7 Hz, 1H).

¹³C NMR (100 MHz, DMSO-d₆) δ 195.62, 173.82, 171.54, 171.04, 169.78, 165.09, 163.98, 158.66 (TFA, q, J=32.4 Hz), 143.28, 137.35, 135.06, 133.37, 132.59 (2C), 132.07, 129.59 (4C), 129.46, 128.57 (2C), 116.80 (TFA, q, J=296.4 Hz), 55.27, 54.51, 52.52, 50.23, 38.73, 38.61, 36.48, 33.79, 33.45, 31.46, 30.84, 30.45, 28.19, 26.62, 26.09, 25.77, 23.07, 22.63.

High resolution mass, calculated [M+H]⁺=758.4236, found 758.4232, Δ=−0.527 ppm.

Formulation of Macrocycle Inhibitors for in vivo Studies.

Purified macrocycle inhibitors were dissolved in DMSO-d₆ (˜200 to 250 mg/mL stock solutions). A sample aliquot (5 μL) of the macrocycle solution was diluted with 445 μL DMSO-d₆, then combined with 50 μL of freshly prepared solution of 20 mM CH₂Cl₂ in DMSO-d₆ in for ¹H-NMR acquisition (600 MHz, relaxation time=2 sec). The inhibitor concentration was calculated using the integral of the CH₂Cl₂ singlet (δ 5.76 ppm, 2H)⁵⁷, which appears in an uncluttered region of these spectra^{51,58}. For injectable formulations (10 mL/kg i.p. injection volume), the macrocycle inhibitor solution in DMSO-d₆ (200-250 mg/mL, based on free-base molecular weight) was combined with 1:20 w/w Captisol® powder (CyDex)⁵⁹. The resulting slurry was supplemented with DMSO-d₆ to make up to 5% of the final formulation volume, mixed thoroughly and dissolved with sterile saline solution (0.9% NaCl). Vehicle controls were identically formulated with 5% DMSO-d₆ and equal amount of Captisol®. The formulated solutions of inhibitor were clear with no visible particles, and were stored overnight at 4° C. prior to injection.

Expression and purification of recombinant cysteine free hIDE (IDE-CF). We expressed cysteine-free catalytically-inactive, human IDE (IDE-CF) in pPROEX vector⁶⁰. IDE-CF contains the following substitutions: C110L, E111Q, C171S, C178A, C257V, C414L, C573N, C590S, C789S, C812A, C819A, C904S, C966N, and C974A. IDE was expressed and purified as previously described⁶⁰. Briefly, IDE-CF was transformed into E. coli BL21-CodonPlus (DE3)-RIL, grown at 37° C. to a cell density of 0.6 O.D. and induced with IPTG at 16° C. for 19 hours. Cells were lysed and the lysate was subjected to Ni-affinity (GE LifeScience) and anion exchange chromatography (GE LifeScience). The protein was further purified by size exclusion chromatography (Superdex S200 column) three successive times, first without inhibitor then two times after addition of 2-fold molar excess of 6b.

IDE-CF6b co-crystallization. Eluent from size exclusion chromatography was concentrated to 20 mg/ml in 20 mM Tris, pH 8.0, 50 mM NaCl, 0.1 mM PMSF and 2-fold molar excess of 6b was added to form the protein-inhibitor complex. The complex was mixed with equal volumes of reservoir solution containing 0.1 M HEPES (pH 7.5), 20% (w/v) PEGMME-5000, 12% tacsimate and 10% dioxane. Crystals appeared after 3-5 days at 25° C. and were then equilibrated in cryoprotective buffer containing well solution and 30% glycerol. IDE-CF6b complex crystals belong to the space group P6₅, with unit-cell dimensions a=b=262 Å and c=90 Å, and contain two molecules of IDE per asymmetric unit (Table 1).

X-Ray Diffraction. X-ray diffraction data were collected at the National Synchrotron Light Source at Brookhaven National Laboratories beamline X29 at 100K and 1.075 Å.

IDE-CF6b structure determination. Data were processed in space group P6₅ using autoProc⁶¹. The structure was phased by molecular replacement using the structure for human IDE E111Q (residues 45-1011, with residues 965-977 missing) in complex with inhibitor compound 41367 (PDB: 2YPU) as a search model in Phaser⁶². The model of the structure was built in Coot⁶³ and refined in PHENIX⁶⁴, using NCS (torsion-angle) and TLS (9 groups per chain). In the Ramachandran plot, 100% of the residues appear in the allowed regions, 97.2% of the residues appear in the favored regions, and 0% of the residues appear in the outlier regions (Table 1). Structure coordinates are deposited in the Protein Data Bank (accession number 4TLE).

Macrocycle docking simulations. Receptor and ligand preparation was performed in the standard method^{65,66}. DOCKing was performed using version 6.6 with default parameters for flexible ligand and grid based scoring except that critical points were used, and the van der Waals exponent was 9. Because of the mutagenesis data strongly pointing to a role of Ala479, we limited docking of the macrocycle to an area within 15 Å of Ala479. The highest scoring poses (FIG. 9), by both gridscore5 and Hawkins GB/SA6, are consistent with the placement of building blocks A and B in the proposed structure.

Anisotropy binding assay. IDE-CF was titrated with fluorescein-labeled macrocycle 31 in 20 mM Tris, pH 8.0, 50 mM NaCl, and 0.1 mM PMSF at 25° C. The final assay volume was 220 μL, with a final DMSO concentration of 0.1% and a final 31 concentration of 0.9 nM. After equilibration, the increase in the fluorescence anisotropy of the fluorescent ligand was recorded at 492 nm, using excitation at 523 nm, and fitted against a quadratic binding equation in Kaleidagraph (Synergy Software) to yield the dissociation constant (K_{D}).

Site-directed mutagenesis, expression, and purification of human IDE. The reported N-His₆-tagged human IDE₄₂₋₁₀₁₉ construct was introduced in the expression plasmid pTrcHis-A (Invitrogen) using primers for uracil-specific excision reactions (USER) by Taq (NEB) and Pfu polymerases (PfuTurbo CX®, Agilent). The IDE gene was amplified with the primers 5′-ATCATCATATGAATAATCCAGCCA-dU-CAAGAGAATAGG and 5′-ATGCTAGCCATACCTCAGA G-dU-TTTGCAGCCATGAAG (underlined sequences represent overhangs, and italics highlight the PCR priming sequence). Similarly, the pTrcHis-A vector was amplified for USER cloning with the primers 5′-ATGGCTGGATTATTCATATGA TGA-dU-GATGATGATGAGAA CCC and 5′-ACTCTGAGGTATGGCTAGCA-dU-GACTGGTG. Mutant IDE constructs were generated by amplifying the full vector construct with USER cloning primers introducing a mutant overhang (Table 5).

All PCR products were purified on microcentrifuge membrane columns (MinElute®, Qiagen) and quantified by UV absorbance (NanoDrop). Each fragment (0.2 pmol) was combined in a 10 μL reaction mixture containing 20 units DpnI (NEB), 0.75 units of USER mix (Endonuclease VIII and Uracil-DNA Glycosylase, NEB), 20 mM Tris-acetate, 50 mM potassium acetate, 10 mM magnesium acetate, 1 mM dithiothreitol at pH 7.9 (1× NEBuffer 4). The reactions were incubated at 37° C. for 45 min, followed by heating to 80° C. and slow cooling to 30° C. (0.2° C./s). The hybridized constructs were directly used for heat-shock transformation of chemically competent NEB turbo E. coli cells according to the manufacturer's instructions. Transformants were selected on carbenicillin LB agar, and isolated colonies were cultured overnight in 2 mL LB.

The plasmid was extracted using a microcentrifuge membrane column kit (Miniprep®, Qiagen), and the sequence of genes and vector junctions were confirmed by Sanger sequencing (Table 6). The plasmid constructs were transformed by heat-shock into chemically-competent expression strain Rosetta 2 (DE3) pLysS E. coli cells (EMD Millipore), and selected on carbenicillin/chloramphenicol LB agar. Cells transformed with IDE pTrcHis A constructs were cultured overnight at 37° C. in 2 XYT media (31 g in 1 L) containing 100 μg/mL ampicillin and 34 μg/mL chloramphenicol. Expression of His6-tagged IDE proteins was induced when the culture measured OD600 ˜0.6 by addition of isopropyl-β-D-1-thiogalactopyranoside (IPTG) to 1 mM final concentration, incubated overnight at 37° C., followed by centrifugation at 10,000 g for 30 min at 4° C.

Recombinant His6-tagged proteins were purified by Ni(II)-affinity chromatography (IMAC sepharose beads, GE Healthcare®) according to the manufacturer's instructions. The cell pellets were resuspended in pH 8.0 buffer containing 50 mM phosphate, 300 mM NaCl, 10 mM imidazole, 1% Triton X-100 and 1 mM tris(2-carboxyethyl)phosphine hydrochloride (TCEP), and were lysed by probe sonication for 4 min at 4° C., followed by clearing of cell debris by centrifugation at 10,000 g for 25 min at 4° C. The supernatant was incubated with Ni(II)-doped IMAC resin (2 mL) for 3 h at 4° C. The resin was washed twice with the cell resuspension/lysis buffer, and three times with pH 8.0 buffer containing 50 mM phosphate, 300 mM NaCl,50 mM imidazole and 1 mM TCEP. Elution was performed in 2 mL aliquots by raising the imidazole concentration to 250 mM and subsequently to 500 mM in the previous buffer. The fractions were combined and the buffer was exchanged to the recommended IDE buffer (R&D) using spin columns with 100 KDa molecular weight cut off membranes (Millipore). Protein yields were typically ˜10 μg/L, and >90% purity based on gel electrophoresis analysis (Coomassie stained). IDE-specific protease activity was >95% as assessed by inhibition of degradation of peptide substrate Mca-RPPGFSAFK(Dnp)-OH (R&D) by 20 μM 6bK, compared with pre-quantitated commercially available human IDE (R&D). The complete list of IDE mutations assayed is shown in Table 7.

In vivo studies, general information. Wild-type C57BL/6J and diet-induced obese (DIO) C57BL/6J age-matched male adult mice were purchased from Jackson Laboratories. The age range was 13 to 15 weeks for lean mice, and 24 to 26 weeks for DIO mice. All animals were individually housed on a 14-h light, 10-h dark schedule at the Biology Research Infrastructure (BRI), Harvard University. Cage enrichment included cotton bedding and a red plastic hut. Water and food were available ad libitum, consisting respectively of normal chow (Prolab® RHM 3000) or high-fat diet (60 kcal % fat, D12492, Research Diets Inc.). All animal care and experimental procedures were in accordance with the standing committee on the Use of Animals in Research and Teaching at Harvard University, the guidelines and rules established by the Faculty of Arts and Sciences' Institutional Animal Care and Use Committee (IACUC), and the National Institutes of Health Guidelines for the Humane Treatment of Laboratory Animals. Glucagon-receptor knock-out (GCGR^{-/-}) mice were housed with a 14-h light and 10-h dark schedule and treated in accordance with the guidelines and rules approved by the IACUC at Albert Einstein College of Medicine, NY. Power analysis to determine animal cohort numbers was based on preliminary results and literature precedent, usually requiring between 5 and 8 animals per group. Animals were only excluded from the cohorts in cases of chronic weakness, which occurs among GCGR −/− mice, or when we identified occasional DIO mice with an outlier diabetic phenotype (>200 mg/dL fasting blood glucose). Age- and weight-matched mice were randomized to each treatment group. Double-blinding was not feasible.

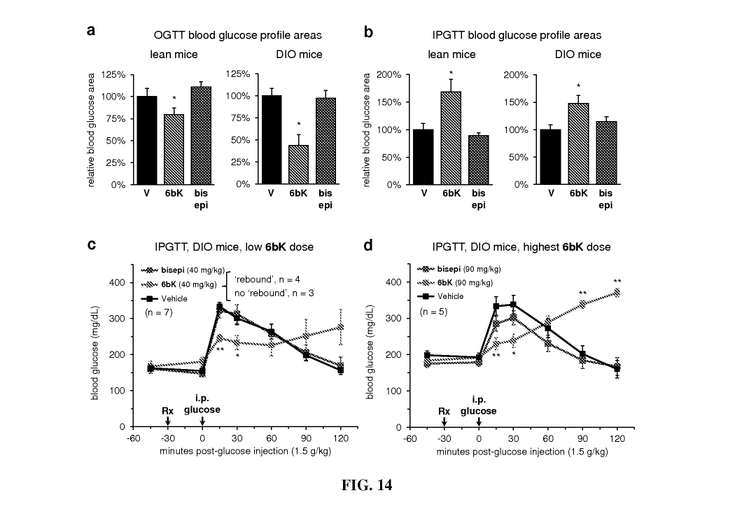

Glucose tolerance tests GTT and blood glucose measurements. Prior to a glucose challenge, the animals were fasted for 14 h (8 pm to 10 am, during the dark cycle) while individually housed in a clean cage with inedible wood-chip as a floor substrate, cotton bedding and a red plastic hut. Inhibitor, vehicle or control compounds were administered by a single intraperitoneal (i.p.) injection 30 min prior to the glucose challenge. Dextrose was formulated in sterile saline (3 g in 10 mL total), and the dose was adjusted by fasted body weight. For oGTT, 3.0 g/kg dextrose was administered by gavage at a dose of 10 mL/kg, and for ipGTT, 1.5 g/kg dextrose injected at a dose of 5 mL/kg. Blood glucose was measured using AccuCheck® meters (Aviva) from blood droplets obtained from a small nick at the tip of the tail, at timepoints −45, 0, 15, 30, 45, 60, 90 and 120 min with reference to the time of glucose injection. The area of the blood glucose response profile curve corresponding to each animal was calculated by the trapezoid method¹⁷, using as reference each individual baseline blood glucose measurement prior to glucose administration (t=0). The sum of the trapezoidal areas between the 0, 15, 30, 45, 60, 90 and 120 minute time points corresponding to each animal were summed to obtain the area under the curve (AUC). The relative area values are expressed as a percentage relative to the average AUC of the vehicle cohort, which is defined as 100% (FIG. 14). Values are reported as mean±S.E.M. Statistics were performed using a two-tail Student's t-test, and significance levels shown in the figures are *p<0.05 versus vehicle control group or **p<0.01 versus vehicle control group.

Insulin Tolerance Test (ITT), Glucagon Challenge (GC) and Amylin Challenge (AC). For hormone challenges animals were fasted individually housed as described above. For ITT the fasting period was 6 h (7 am to 1 pm), and for glucagon and amylin challenges the fasting period was 14 h (8 pm-10 am). Inhibitor or vehicle alone was injected i.p. as previously described, 30 min prior to each hormone challenge. Insulin (Humulin-R®, Eli Lilly) was injected subcutaneously (s.c.) 0.25 U/kg formulated in sterile saline (5 mL/kg). Glucagon (Eli Lilly) was injected s.c. 100 μg/kg formulated in 0.5% BSA sterile saline (5 mL/kg). Amylin (Bachem) was injected s.c. 250 μg/kg formulated in sterile saline (5 mL/kg). Blood glucose was measured at timepoints −45, 0, 15, 30, 45, 60 and 75 min with reference to the time of hormone injection, by microsampling from a tail nick as described above. Values are reported as mean±S.E.M. Statistics were performed using a two-tail Student's t-test, and significance levels shown in the figures are *p<0.05 versus vehicle control group or **p<0.01 versus vehicle control group.

Blood collection and plasma hormone measurements. Blood was collected in EDTA-coated tubes (BD Microtainer®) from trunk bleeding (˜500 μL) after CO₂-euthanasia for all hormone assays. The plasma was immediately separated from red blood cells by centrifugation 10 min at 1800 g, aliquoted, frozen over dry ice and stored at −80° C. Insulin, glucagon, amylin and pro-insulin C-peptide fragment were quantified from 10 μL plasma samples using magnetic-bead Multiplexed Mouse Metabolic Hormone panel (Milliplex, EMD Millipore) according to the manufacturer's instructions, using a Luminex FlexMap 3D instrument. Plasma containing high levels of human insulin (Humulin-R) were quantified using 25 μL samples with Insulin Ultrasensitive ELISA (ALPCO). Values are reported as mean±S.E.M. Statistics were performed using a two-tail Student's t-test, and significance levels shown in the figures are *p<0.05 versus vehicle control group or **p<0.01 versus vehicle control group.

Gastric emptying measurements. Mice were fasted 14 h (8 pm to 10 am). Inhibitor or vehicle alone was injected i.p. as previously described, followed 30 min later by an oral glucose bolus (3.0 g/kg, 10 mL/kg) in sterile saline containing 0.1 mg/mL phenol red. At 30 min, the stomach was promptly dissected after CO₂-euthanasia and stored on ice. The stomach contents were extracted into 2.5 mL EtOH (95%) by homogenization for 1 min using a probe sonicator. Tissue debris were decanted by centrifugation at 4000 g, followed by clearing at 15000 g for 15 min. The supernatant (1 mL) was mixed with 0.5 mL of aqueous NaOH (20 mM), and incubated at −20° C. for 1 h. The solution was centrifuged at 15,000 g for 15 min, and absorbance was determined at 565 nM. The spectrophotometer was blanked with the stomach contents of a mouse treated with colorless glucose solution. The absorbance was adjusted to the amount of glucose solution dosed to each mouse. Values are reported as mean±S.E.M relative to the original phenol red glucose solution. Statistics were performed using a two-tail Student's t-test, and significance levels shown in the figures are *p<0.05 versus vehicle control group or **p<0.01 versus vehicle control group.

Stable isotope dilution LC-MS, pharmacokinetics, and tissue distribution measurements. Heavy-labeled macrocycle inhibitor (heavy-6bK, FIG. 13) was synthesized as described above, substituting “building block C” with N^{α}-Fmoc-N^{c}-Boc-lysine ¹³C₆¹⁵N₂ (98 atom %, Sigma-Aldrich). The product was 8 mass-units heavier than 6bK, otherwise with identical properties and IC₅₀. Plasma samples (15 μL) from 6bK-treated mice and vehicle controls were combined with 5 μL of heavy-6bK in PBS (final concentration of 10 μM), and incubated for 30 min on ice. Plasma proteins were precipitated with 180 μL cold 1% TFA in MeCN, sonicated 2 min, and centrifuged 13,000 g for 1 min. The supernatant was diluted 100- and 1000-fold for liquid chromatography-mass spectrometry (LC-MS) analysis. Tissue samples (˜100 mg) fromb 6K-treated mice and vehicle controls were weighed and disrupted in Dounce homogenizers with PBS buffer (0.5 mL/100 mg sample), supplemented with 5 μM heavy-6bK and protease inhibitor cocktail (1 tablet/50 mL PBS, Roche diagnostics). The lysate was incubated on ice for 30 min, sonicated 5 min and centrifuged at 13,000 g for 5 min. The bulk of supernatant proteins were precipitated by denaturation at 95° C. for 5 min, removed by centrifugation. A 50 μL aliquot of the supernatant was treated with 450 μL cold 1% TFA in MeCN, cleared by centrifugation and diluted 100-fold for LC-MS analysis as described above for plasma samples. A standard curve of 6bK and heavy-6bK (1 μM each and 3-fold serial dilutions) were used for LC-MS quantitation using a Waters Q-TOF premier instrument.

RT-PCR analysis of liver gluconeogenesis markers phosphoenolpyruvate carboxykinase (PEPCK) and glucose-6-phosphatase (G6Pase). Total RNA was isolated from liver samples (˜100 mg) using TRIzol® reagent (Invitrogen, 1 mL/100 mg sample), followed by spin-column purification (RNeasy® kit, Qiagen) and on-column DNAse treatment (Qiagen) according to the manufacturer's instructions. RNA concentrations were determined by UV spectrophotometry (NanoDrop). One microgram of total RNA was used for reverse transcription with oligo(dT) primers (SuperScript® III First-Strand Synthesis SuperMix, Life Technologies) according to the manufacturer's instructions. Quantitative PCR reactions included 1 μL of cDNA diluted 1:100, 0.4 μM primers, 2× SYBR Green PCR Master Mix (Invitrogen) in 25 μL total volume, and were read out by a CFX96TM Real-Time PCR Detection System (BioRad). Transcript levels were determined using two known primer pairs^{18,19} for each gene of interest (Table 6), which were normalized against tubulin and beta-actin transcripts (ΔΔC_{T} method), and expressed relative to the lowest sample. Duplicate control assays without reverse transcriptase treatment were run for each RNA preparation and primer set used. Statistics were performed using a two-tail Student's t-test, and significance levels shown in the figures are *p<0.05 versus vehicle control group or **p<0.01 versus vehicle control group.

TABLE 1
Data collection and refinement statistics (molecular replacement).
One crystal was used to solve the CF-IDE•6b structure. Highest-
resolution shell is shown in parentheses. Structure coordinates
are deposited in the Protein Data Bank (accession number 4TLE).

| Data collection | hIDE-CF•6b |
|---|---|
| Space group | P6₅ |
| Cell dimensions | |
| a, b, c (Å) | 261.97, 261.97, 90.8 |
| (°) | 90, 90, 120 |
| Resolution (Å) | 130.99-2.70 |
| R_{merge} | 0.135 (0.683) |
| I/I | 14.8 (2.4) |
| Completeness (%) | 100 (99.9) |
| Redundancy | 7.4 |
| Refinement | |
| Resolution (Å) | 2.705 |
| No. reflections | 97260 |
| R_{work}/R_{free} | 0.1634/0.1986 |
| No. atoms | |
| Protein | 15531 |
| Ligand/ion | 160 |
| Water | 563 |
| B-factors | |
| Protein | Chain A: 30.23 |
| | Chain B: 33.51 |
| Ligand/ion | Chain A: 58.42 |
| | Chain B: 59.53 |
| Water | 31.67 |
| R.m.s. deviations | |
| Bond lengths (Å) | 0.014 |
| Bond angles (°) | 1.395 |
| PDB accession code | 4LTE |

TABLE 2
Candidate IDE substrates identified using
in vitro assays and mass spectrometry.

| | Degra- | Confirmed in |
|---|---|---|
| IDE substrates in vitro (references) | dation* | vivo (references) |
| insulin (4) | Fast | KO studies (10, |
| | | 11), this study |
| amyloid-β peptide_{(40/42)} † | Fast | KO study (10) |
| calcitonin-gene related peptide (CGRP) | Medium | KO study (18) |
| glucagon (35, 36) | Slow | this study |
| amylin (37) | Medium | this study |
| tissue growth factor-α † | Fast | — |
| insulin-like growth factor-2 † | Fast | — |
| insulin-like growth factor-1 † | Slow | — |
| somatostatin-14 † | Slow | — |
| bradykinin † | Slow | — |
| kallidin † | Slow | — |
| atrial natriuretic peptide (ANP) † | Fast | — |
| B-type natriuretic peptide (BNP) † | Slow | — |
| relaxin † | Fast | — |
| relaxin-3 † | Fast | — |
| insulin-like peptide 3 † | Fast | — |
| β-endorphin₍₁₋₃₁₎ † | Slow | — |
| growth-hormone releasing factor₍₁₋₂₉₎ † | Slow | — |
| pancreastatin₍₁₋₄₉₎ † | Slow | — |
| dynorphins A and B † | Slow | — |

*Degradation kinetics compared to insulin; see Table 3 for detailed kinetic parameters. Known non-substrates include glucagon-like peptide 1 (GLP-1), glucose-dependent insulinotropic peptide (GIP), epithelial growth factor 1 (EGF-1) and C-peptide.
† References are shown in Table 3.

TABLE 3
Literature survey of putative IDE substrates identified using in vitro assays.

| IDE substrates | Alternative degrading | k_{cat} | K_{M} | k_{cat}/ | IC₅₀ | X-ray | IDE substrate |
|---|---|---|---|---|---|---|---|
| in vitro (reference) | enzymes (Brenda db) | (min⁻¹) | (μM) | K_{M} | (μM) | struct. | in vivo (ref.) |
| Insulin ^{22,23} | lysomal proteases, | 1.52 | <0.03 | 50 | — | 2WBY | KO study ^{22,23} |
| | disulfide reductase | | | | | 2WC0 | |
| Aβ (40, 42) ^{24,25} | NEP, cathepsin D | 52 | 1.23 | 43 | — | 2G47 | KO study ^{22,26,27} |
| | | | | | | 2WK3 | |
| calcitonin-gene | ECE, PREP | — | — | — | — | — | KO study ⁶ |
| related peptide⁶ | | | | | | | |
| glucagon ^{28,29} | NEP, nardilysin, | 38.5 | 3.46 | 11 | weak | 2G49 | this study |
| | DPP-IV, cathepsins | | | | | | |
| | B and C | | | | | | |
| amylin ³⁰ | — | n/a | 0.3* | n/a | 0.16 | 3HGZ | this study |
| | | | | | | 2G48 | |
| TGF-α ³¹ | — | — | 0.15* | — | 0.08 | 3E50 | — |
| IGF-1 ^{26,32} | DPP-IV | — | Weak | — | — | — | — |
| IGF-2 ^{26,32} | — | — | 0.1* | — | 0.06 | 3E4Z | — |
| somatostatin-14 ³³ | nardilysin, dactylysin, | 22.8 | 7.5 | 3.0 | — | — | — |
| | aminopeptidase B, | | | | | | |
| | THOP, neurotensin- | | | | | | |
| | degrading enzyme | | | | | | |
| bradykinin ³⁴ | ACE, aminopeptidase | — | 4.2 | — | — | 3CWW | — |
| | P, carboxypeptidase N, | | | | | | |
| | NEP, DPP-II, DPP-IV, | | | | | | |
| kallidin ³⁴ | ACE, aminopeptidase | — | 7.3 | — | — | — | — |
| | P, carboxypeptidase N | | | | | | |
| atrial NP (ANP) ^{35,36} | NEP | — | 0.06 | — | — | 3N57 | — |
| B-type NP (BNP) ³⁶ | NEP, fibroblast | — | weak | — | >1 | 3N56 | — |
| | activation protein-α | | | | | | |
| relaxin ³⁷ | — | — | 0.11* | — | 0.054 | — | — |
| relaxin-3 ³⁷ | — | — | 0.36* | — | 0.182 | — | — |
| insulin-like peptide 3 ³⁸ | — | 0.15 | 0.055 | 2.7 | — | — | — |
| β-endorphin (1-31) ³⁴ | NEP | 21 | 13 | 1.6 | — | — | — |
| growth hormone | DPP-IV, NEP | 23.4 | 11.9 | 2.0 | — | — | — |
| releasing factor (1-29) ³⁴ | | | | | | | |
| pancreastatin (1-49) ³⁴ | — | 10.6 | 41.7 | 0.25 | — | — | — |
| dynorphin B (1-13) ³⁴ | NEP | 15.1 | 18.3 | 0.8 | — | — | — |
| A (1-17) ³⁴ | NEP | 18.4 | 37.4 | 0.5 | — | — | — |
| B (1-9) ³⁴ | NEP | 10.3 | 26.8 | 0.4 | — | — | — |
| A (1-13) ³⁴ | NEP | 3.74 | 40.6 | 0.09 | — | — | — |
| A (1-10) ³⁴ | NEP | 3.74 | 39.4 | 0.09 | — | — | — |
| A (1-8) ³⁴ | NEP | 0.66 | 63.5 | 0.01 | — | — | — |
| A (1-9) ³⁴ | NEP | 0.33 | 60.5 | 0.01 | — | — | — |

*K_{M} estimated based on IC₅₀ for inhibition of insulin degradation.
Abbreviations:
Aβ = amyloid beta;
TGF-alpha = transforming growth factor-alpha;
IGF = insulin-like growth factor;
NP = natriuretic peptide;
GRF = gastrin release factor;
NEP = neprilysin;
PREP = prolyl endopeptidase;
ECE = endothelin converting enzyme;
TROP = thimet oligopeptidase;
ACE = angiotensin-converting enzyme;
DPP = dipeptidylpeptidase.
X-ray structure accession numbers are provided for the RCSB Protein Data Bank (pdb[dot]org).

TABLE 4
HPLC and high-resolution mass spectrometry analysis of IDE inhibitor analogs.

| | Purity | | [M + H] + | [M + H] + | Ion | |
|---|---|---|---|---|---|---|
| Compound | (%) | Formula | expected | found | count | Δ (ppm) |
| 1a | 87.5 | C₂₆H₄₁N₇O₇ | 564.3140 | 564.3135 | 17505 | −0.886 |
| 1b | 66.4 | C₂₆H₄₁N₇O₇ | 564.3140 | 564.3141 | 237080 | 0.177 |
| 2a | 84.1 | C₄₁H₅₅N₉O₇ | 786.4297 | 786.4290 | 523007 | −0.890 |
| 2b | 95.3 | C₄₁H₅₅N₉O₇ | 786.4297 | 786.4272 | 627894 | −3.179 |
| 3a | 84.3 | C₄₆H₆₂N₆O₈ | 827.4702 | 827.4703 | 111258 | 0.121 |
| 3b | 89.9 | C₄₆H₆₂N₆O₈ | 827.4702 | 827.4703 | 26066 | 0.121 |
| 4a | 93.7 | C₃₂H₅₂N₆O₇ | 633.3970 | 633.3975 | 1344574 | 0.789 |
| 4b | 79.7 | C₃₂H₅₂N₆O₇ | 633.3970 | 633.3967 | 591800 | −0.474 |
| 5a | 97.1 | C₄₁H₅₂N₆O₇ | 741.3970 | 741.3987 | 517852 | 2.293 |
| 5b | 75.4 | C₄₁H₅₂N₆O₇ | 741.3970 | 741.3967 | 208124 | −0.405 |
| 6a | 98.3 | C₄₀H₅₁N₇O₈ | 758.3872 | 758.3886 | 875721 | 1.846 |
| 6b | 98.9 | C₄₀H₅₁N₇O₈ | 758.3872 | 758.3878 | 14154 | 0.791 |
| 7 | 89.6 | C₄₀H₅₃N₇O₇ | 744.4079 | 744.4079 | 21411 | −0.040 |
| 8 | 94.9 | C₃₃H₄₇N₇O₇ | 654.3610 | 654.3606 | 413032 | −0.611 |
| 9 | 80.1 | C₃₇H₅₅N₇O₇ | 710.4236 | 710.4239 | 180420 | 0.422 |
| 10 | 76.9 | C₃₉H₅₁N₇O₇ | 730.3923 | 730.3917 | 644559 | −0.821 |
| 11 | 92.6 | C₄₀H₄₅N₇O₈ | 752.3402 | 752.3399 | 77585 | −0.399 |
| 12 | 96.0 | C₃₇H₄₇N₇O₈ | 718.3559 | 718.3550 | 145796 | −1.253 |
| 13 | 95.7 | C₃₇H₄₇N₇O₉ | 718.3559 | 718.3569 | 24610 | 1.392 |
| 14 | 58.2 | C₃₄H₄₁N₇O₈ | 676.3089 | 676.3082 | 400965 | −1.035 |
| 15 | 77.5 | C₃₈H₄₈N₆O₇ | 701.3657 | 701.3671 | 124002 | 1.996 |
| 16 | 67.3 | C₃₈H₄₈N₆O₇ | 701.3657 | 701.3683 | 19335 | 3.707 |
| 17 | 83.2 | C₃₉H₅₀N₆O₇ | 715.3814 | 715.3808 | 298036 | −0.839 |
| 18 | 92.3 | C₃₉H₅₀N₆O₇ | 715.3814 | 715.3832 | 701250 | 2.516 |
| 19 | 37.4 | C₃₈H₄₈N₆O₈ | 717.3606 | 717.3586 | 128261 | −2.788 |
| 20 | 74.5 | C₃₇H₄₆N₆O₇ | 687.3501 | 687.3503 | 135532 | 0.291 |
| 21 | 95.6 | C₄₀H₅₀N₆O₉ | 759.3712 | 759.3726 | 13461 | 1.844 |
| 22 | 97.9 | C₄₀H₅₂N₆O₈S | 777.3640 | 777.3634 | 121542 | −0.772 |
| 23 | 88.5 | C₄₀H₅₂N₆O₇Se | 807.3144 | 807.3139 | 1497 | −0.619 |
| 24 | 52.4 | C₄₄H₅₂N₆O₇ | 777.3970 | 777.3981 | 15952 | 1.415 |
| 25 | 63.5 | C₃₅H₄₃N₅O₈ | 630.3286 | 630.3282 | 136628 | −0.635 |
| 26 | 74.4 | C₄₀H₅₁N₇O₈ | 758.3872 | 758.3876 | 119065 | 0.527 |
| 27 | 90.9 | C₃₉H₄₉N₇O₈ | 744.3715 | 744.3708 | 210827 | −0.940 |
| 29 | 75.3 | C₄₂H₅₅N₇O₈ | 786.4185 | 786.4178 | 234651 | −0.890 |
| 28 | 94.3 | C₄₀H₅₀N₆O₉ | 759.3712 | 759.3705 | 89863 | −0.922 |
| 30 | 97.5 | C₅₄H₇₅N₉O₁₁S | 1058.5380 | 1058.5402 | 23247 | 2.078 |
| 31 | 81.6 | C₆₅H₇₁N₇O₁₅ | 1190.5081 | 1190.5063 | 442 | −1.512 |
| 6bK | 96.6 | C₄₁H₅₅N₉O₇ | 758.4233 | 758.4233 | 501430 | −0.396 |
| heavy 6bK | 96.4 | C₃₅¹³C₆H₅₅N₅ ¹⁵O₇ | 766.4378 | 766.4388 | 227096 | 1.305 |
| bisepi-6bK | 96.7 | C₄₁H₅₅N₇O₇ | 758.4236 | 758.4232 | 133009 | −0.527 |
| epiA-6bK | 96.6 | C₄₁H₅₅N₇O₇ | 758.4236 | 758.4258 | 1320288 | 2.901 |
| epiB-6bK | 94.9 | C₄₁H₅₅N₇O₇ | 758.4236 | 758.4235 | 216951 | −0.132 |
| epiC-6bK | 93.9 | C₄₁H₃₅N₇O₇ | 758.4236 | 758.4235 | 177995 | −0.132 |
| Ii1 | 94.8 | C₃₇H₄₅N₉O₈ | 744.3464 | 74.3474 | 662 | 1.343 |

TABLE 5
Site-directed mutagenesis primers.

| Mutation | Primers (forward, reverse) |
|---|---|
| A198Q | ACATGAGAAGAATGTGATGAATGA-dU-CAGTGGAGAC |
| | ATCATTCATCACATTCTTCTCATG-dU-TCTG |
| A198T | AGACTCTTTCAATTGGAAAAAGC-dU-ACAGGG |
| | AGCTTTTTCCAATTGAAAGAGTC-dU-CCAGGTATCATTCATCACATTCTTCTCATGTTC |
| A198C | AGACTCTTTCAATTGGAAAAAGC-dU-ACAGGG |
| | AGCTTTTTCCAATTGAAAGAGTC-dU-CCAGCAATCATTCATCACATTCTTCTCATGTTC |
| A198Y | ACATGAGAAGAATGTGATGAATGA-dU-TACTGGAGAC |
| | ATCATTCATCACATTCTTCTCATG-dU-TCTG |
| W199L | ACATGAGAAGAATGTGATGAATGA-dU-GCCTTAAGACTC |
| | ATCATTCATCACATTCTTCTCATG-dU-TCTG |
| W199F | AGACTCTTTCAATTGGAAAAAGC-dU-ACAGG |
| | AGCTTTTTCCAATTGAAAGAGTC-dU-GAAGGCATCATTCATCACATTCTTCTC |
| W199Y | AGACTCTTTCAATTGGAAAAAGC-dU-ACAGGG |
| | AGCTTTTTCCAATTGAAAGAGTC-dU-ATAGGCATCATTCATCACATTCTTCTC |
| F202L | ACATGAGAAGAATGTGATGAATGA-dU-GCCTGGAGACTCTTGCAATTG |
| | ATCATTCATCACATTCTTCTCATG-dU-TCTG |
| F202R | AGACTCTTTCAATTGGAAAAAGC-dU-ACAGGG |
| | ATGAATGATGCCTGGAGAC-dU-CCGTCAATTGGAAAAAGCTACAGGG |
| I310R | ACCCATTAAAGATCGTAGGAATC-dU-CTATGTGACATTTCCCATAC |
| | AGATTCCTACGATCTTTAATGGG-dU-ACTATTTTGTAAAGTTGTTTAAG |
| Y314F | ACCCATTAAAGATATTAGGAATCTC-dU-TCGTGACATTTCCCATACCTGACCTTC |
| | AGAGATTCCTAATATCTTTAATGGG-dU-ACTATTTTG |
| V3602Q | AAAGGGCTGGGTTAATACTCT-dU-CAGGGTGGGCAG |
| | AAGAGTATTAACCCAGCCCTT-dU-GACTTAAG |
| V360R | AAAGGGCTGGGTTAATACTCT-dU-AGGGGTGGGCAGAAGGAAGGAGC |
| | AAGAGTATTAACCCAGCCCTT-dU-GACTTAAG |
| G361Q | AAAGGGCTGGGTTAATACTCT-dU-GTTCAGGGGCAGAAGGAAGGAGCCC |
| | AAGAGTATTAACCCAGCCCTT-dU-GACTTAAG |
| G362Q | AAAGGGCTGGGTTAATACTCT-dU-GTTGGTCAGCAGAAGGAAGGAGCCCGAG |
| | AAGAGTATTAACCCAGCCCTT-dU-GACTTAAG |
| K364A | AAGGAGCCCGAGGTTTTA-dU-GTTTTTTATC |
| | ATAAAACCTCGGGCTCCT-dU-CCGCCTGCCCACCAACAAGAGTATT |
| I374M | ATGTTTTTTATCATGAATGTGGACT-dU-GACCG |
| | AAGTCCACATTCATGATAAAAAAC-dU-AAAACCTC |
| I374Q | ATGTTTTTTATCCAGAATGTGGACT-dU-GACCGAGGAAGG |
| | AAGTCCACATTCTGGATAAAAAACA-dU-AAAACCTCGGGCTCC |
| A479L | ATGTCCGGGTTCTGATAGTTTCTAAA-dU-CTTTTGAAGGAAAAACTG |
| | ATTTAGAAACTATCAGAACCCGGACA-dU-TTTCTGGTCTGAG |

TABLE 6
Sequencing and RT-PCR primers.
Sequencing primers

| | |
|---|---|
| Seq_Fw1 | GATTAACTTTATTATTAAAAATTAAAGAGG |
| Seq_Re1 | CAACATGTAATAATCCTTCCTCGGTC |
| Seq_Fw2 | GCATGAAGGTCCTGGAAGTCTG |
| Seq_Re2 | AGGAAGGGTTACATCATCCAGAGC |
| Seq_Fw3 | CCATGTACTACCTCCGCTTGC |
| Seq_Re3 | GCAGATCTCGAGCTCGGATC |
| Seq_Fw4 | GCTTATGTGGACCCCTTGCACTG |

RT-PCR primers^{18, 19}

| | |
|---|---|
| PEPCK_1_Fw | GAACTGACAGACTCGCCCTATGT |
| PEPCK_1_Re | GTTGCAGGCCCAGTTGTTG |
| G6Pase_1_Fw | CTGCAGCTGAACGTCTGTCTGT |
| G6Pase_1_Re | TCCGGAGGCTGGCATTGT |
| PEPCK_2_Fw | GGTGTTTACTGGGAAGGCATC |
| PEPCK_2_Re | CAATAATGGGGCACTGGCTG |
| G6Pase_2_Fw | CATGGGCGCAGCAGGTGTATACT |
| G6Pase_2_Re | CAAGGTAGATCCGGGACAGACAG |
| Tubulin_Fw | CCTGCTCATCAGCAAGATCC |
| Tubulin_Re | TCTCATCCGTGTTCTCAACC |
| Beta-actin_Fw | CATCCGTAAAGACCTCTATGCCAAC |
| Beta-actin_Re | ATGGAGCCACCGATCCACA |

TABLE 7
Site-directed IDE mutants used in the small molecule-enzyme
mutant complementation studies.

| | | Relative | Ii1 IC₅₀ | 6b IC₅₀ | 13 IC₅₀ | 9 IC₅₀ | |
|---|---|---|---|---|---|---|---|
| Protein | Batch | activity | shift * | shift | shift | shift | Prediction |
| hIDE WT | 1 | 1.0 | 1.0 | 1.0 | 1.0 | 1.0 | |
| hIDE WT | 2 | 1.0 | 1.0 | 1.0 | 1.0 | — | |
| hIDE WT | 3 | 1.0 | 1.0 | 1.0 | 1.0 | — | |
| A198Q | 1 | 1.4 | 1.0 | 2.3 | — | — | |
| A198T | 2 | 1.5 | 1.0 | 16 | 13.6 | — | scaffold interaction |
| A198Y | 2 | 2.3 | 1.1 | 0.5 | — | — | |
| W199F | 2 | 1.3 | 1.8 | 3.0 | 3.2 | — | modest interaction |
| W199Y | 2 | 0.3 | 1.4 | 1.8 | — | — | |
| F202L | 1 | 0.7 | 1.7 | 1.7 | 2.3 | — | |
| F202R | 2 | 0.9 | 1.1 | 3.7 | 4.1 | — | modest interaction |
| I310R | 2 | 3.0 | 1.9 | 0.8 | 0.7 | — | |
| Y314F | 2 | 3.5 | 1.0 | 1.2 | — | — | |
| V360Q | 1 | 2.6 | 0.9 | 1.5 | — | — | |
| V360R | 3 | 0.4 | 0.9 | 1.4 | — | — | |
| G361Q | 3 | 0.7 | 0.9 | 0.8 | — | — | |
| G362Q | 3 | 1.2 | 0.6 | 77 | 1.8 | — | cyclohexyl ring interaction |
| K364A | 2 | 4.3 | 1.0 | 26 | 10.0 | — | scaffold interaction |
| I374M | 1 | 4.3 | 1.0 | 0.7 | — | — | |
| A479L | 1 | 0.5 | 0.9 | >600 | — | 1.0 | p-benzoyl interaction |

* NB: Ii1 is not known to interact with any of these residues.
Significant changes in IC₅₀ for Ii1 were presumed to indicate misfolding or other non-inhibitor-specific protein structure changes. For example, W199L displayed an IC₅₀ shift of 21-fold for Ii1, 13-fold for 6b, and 17-fold for analog 13.

pTrcHisA-His₆-hIDE(42-1019) plasmid sequence
(IDE coding sequence is in underlined)
(SEQ ID NO: XX)
GTTTGACAGCTTATCATCGACTGCACGGTGCACCAATG
CTTCTGGCGTCAGGCAGCCATCGGAAGCTGTGGTATG
GCTGTGCAGGTCGTAAATCACTGCATAATTCGTGTCGC
TCAAGGCGCACTCCCGTTCTGGATAATGTTTTTTGCG
CCGACATCATAACGGTTCTGGCAAATATTCTGAAATGA
GCTGTTGACAATTAATCATCCGGCTCGTATAATGTGT
GGAATTGTGAGCGGATAACAATTTCACACAGGAAACAG
CGCCGCTGAGAAAAAGCGAAGCGGCACTGCTCTTTAA
CAATTTATCAGACAATCTGTGTGGGCACTCGACCGGAA
TTATCGATTAACTTTATTATTAAAAATTAAAGAGGTA
TATATTAATGTATCGATTAAATAAGGAGGAATAAACCA
TGGGGGGTTCTCATCATCATCATCATCATATGAATAA
TCCAGCCATCAAGAGAATAGGAAATCACATTACCAAGT
CTCCTGAAGACAAGCGAGAATATCGAGGGCTAGAGCT
GGCCAATGGTATCAAAGTACTTCTTATCAGTGATCCCA
CCACGGATAAGTCATCAGCAGCACTTGATGTGCACAT
AGGTTCATTGTCGGATCCTCCAAATATTGCTGGCTTAA
GTCATTTTTGTGAACATATGCTTTTTTTGGGAACAAA
GAAATACCCTAAAGAAAATGAATACAGCCAGTTTCTCA
GTGAGCATGCAGGAAGTTCAAATGCCTTTACTAGTGG
AGAGCATACCAATTACTATTTTGATGTTTCTCATGAAC
ACCTAGAAGGTGCCCTAGACAGGTTTGCACAGTTTTT
TCTGTGCCCCTTGTTCGATGAAAGTTGCAAAGACAGAG
AGGTGAATGCAGTTGATTCAGAACATGAGAAGAATGT
GATGAATGATGCCTGGAGACTCTTTCAATTGGAAAAAG
CTACAGGGAATCCTAAACACCCCTTCAGTAAATTTGG
GACAGGTAACAAATATACTCTGGAGACTAGACCAAACC
AAGAAGGCATTGATGTAAGACAAGAGCTACTGAAATT
CCATTCTGCTTACTATTCATCCAACTTAATGGCTGTTT
GTGTTTTAGGTCGAGAATCTTTAGATGACTTGACTAA
TCTGGTGGTAAAGTTATTTTCTGAAGTAGAGAACAAAA
ATGTTCCATTGCCAGAATTTCCTGAACACCCTTTCCA
AGAAGAACATCTTAAACAACTTTACAAAATAGTACCCA
TTAAAGATATTAGGAATCTCTATGTGACATTTCCCAT
ACCTGACCTTCAGAAATACTACAAATCAAATCCTGGTC
ATTATCTTGGTCATCTCATTGGGCATGAAGGTCCTGG
AAGTCTGTTATCAGAACTTAAGTCAAAGGGCTGGGTTA
ATACTCTTGTTGGTGGGCAGAAGGAAGGAGCCCGAGG
TTTTATGTTTTTTATCATTAATGTGGACTTGACCGAGG
AAGGATTATTACATGTTGAAGATATAATTTTGCACAT
GTTTCAATACATTCAGAAGTTACGTGCAGAAGGACCTC
AAGAATGGGTTTTCCAAGAGTGCAAGGACTTGAATGC
TGTTGCTTTTAGGTTTAAAGACAAAGAGAGGCCACGGG
GCTATACATCTAAGATTGCAGGAATATTGCATTATTA
TCCCCTAGAAGAGGTGCTCACAGCGGAATATTTACTGG
AAGAATTTAGACCTGACTTAATAGAGATGGTTCTCGA
TAAACTCAGACCAGAAAATGTCCGGGTTGCCATAGTTT
CTAAATCTTTTGAAGGAAAAACTGATCGCACAGAAGA
GTGGTATGGAACCCAGTACAAACAAGAAGCTATACCGG
ATGAAGTCATCAAGAAATGGCAAAATGCTGACCTGAA
TGGGAAATTTAAACTTCCTACAAAGAATGAATTTATTC
CTACGAATTTTGAGATTTTACCGTTAGAAAAAGAGGC
GACACCATACCCTGCTCTTATTAAGGATACAGCTATGA
GCAAACTTTGGTTCAAACAAGATGATAAGTTTTTTTT
GCCGAAGGCTTGTCTCAACTTTGAATTTTTCAGCCCAT
TTGCTTATGTGGACCCCTTGCACTGTAACATGGCCTA
TTTGTACCTTGAGCTCCTCAAAGACTCACTCAACGAGT
ATGCATATGCAGCAGAGCTAGCAGGCTTGAGCTATGA
TCTCCAAAATACCATCTATGGGATGTATCTTTCAGTGA
AAGGTTACAATGACAAGCAGCCAATTTTACTAAAGAA
GATTATTGAGAAAATGGCTACCTTTGAGATTGATGAAA
AAAGATTTGAAATTATCAAAGAAGCATATATGCGATC
TCTTAACAATTTCCGGGCTGAACAGCCTCACCAGCATG
CCATGTACTACCTCCGCTTGCTGATGACTGAAGTGGC
CTGGACTAAAGATGAGTTAAAAGAAGCTCTGGATGATG
TAACCCTTCCTCGCCTTAAGGCCTTCATACCTCAGCT
CCTGTCACGGCTGCACATTGAAGCCCTTCTCCATGGAA
ACATAACAAAGCAGGCTGCATTAGGAATTATGCAGAT
GGTTGAAGACACCCTCATTGAACATGCTCATACCAAAC
CTCTCCTTCCAAGTCAGCTGGTTCGGTATAGAGAAGT
TCAGCTCCCTGACAGAGGATGGTTTGTTTATCAGCAGA
GAAATGAAGTTCACAATAACTGTGGCATCGAGATATA
CTACCAAACAGACATGCAAAGCACCTCAGAGAATATGT
TTCTGGAGCTCTTCTGTCAGATTATCTCGGAACCTTG
CTTCAACACCCTGCGCACCAAGGAGCAGTTGGGCTATA
TCGTCTTCAGCGGGCCACGTCGAGCTAATGGCATACA
GGGCTTGAGATTCATCATCCAGTCAGAAAAGCCACCTC
ACTACCTAGAAAGCAGAGTGGAAGCTTTCTTAATTAC
CATGGAAAAGTCCATAGAGGACATGACAGAAGAGGCCT
TCCAAAAACACATTCAGGCATTAGCAATTCGTCGACT
AGACAAACCAAAGAAGCTATCTGCTGAGTGTGCTAAAT
ACTGGGGAGAAATCATCTCCCAGCAATATAATTTTGA
CAGAGATAACACTGAGGTTGCATATTTAAAGACACTTA
CCAAGGAAGATATCATCAAATTCTACAAGGAAATGTT
GGCAGTAGATGCTCCAAGGAGACATAAGGTATCCGTCC
ATGTTCTTGCCAGGGAAATGGATTCTTGTCCTGTTGT
TGGAGAGTTCCCATGTCAAAATGACATAAATTTGTCAC
AAGCACCAGCCTTGCCACAACCTGAAGTGATTCAGAA
CATGACCGAATTCAAGCGTGGTCTGCCACTGTTTCCCC
TTGTGAAACCACATATTAACTTCATGGCTGCAAAACT
CTGAGGTATGGCTAGCATGACTGGTGGACAGCAAATGG
GTCGGGATCTGTACGACGATGACGATAAGGATCGATG
GGGATCCGAGCTCGAGATCTGCAGCTGGTACCATATGG
GAATTCGAAGCTTGGCTGTTTTGGCGGATGAGAGAAG
ATTTTCAGCCTGATACAGATTAAATCAGAACGCAGAAG
CGGTCTGATAAAACAGAATTTGCCTGGCGGCAGTAGC
GCGGTGGTCCCACCTGACCCCATGCCGAACTCAGAAGT
GAAACGCCGTAGCGCCGATGGTAGTGTGGGGTCTCCC
CATGCGAGAGTAGGGAACTGCCAGGCATCAAATAAAAC
GAAAGGCTCAGTCGAAAGACTGGGCCTTTCGTTTTAT
CTGTTGTTTGTCGGTGAACGCTCTCCTGAGTAGGACAA
ATCCGCCGGGAGCGGATTTGAACGTTGCGAAGCAACG
GCCCGGAGGGTGGCGGGCAGGACGCCCGCCATAAACTG
CCAGGCATCAAATTAAGCAGAAGGCCATCCTGACGGA
TGGCCTTTTTGCGTTTCTACAAACTCTTTTTGTTTATT
TTTCTAAATACATTCAAATATGTATCCGCTCATGAGA
CAATAACCCTGATAAATGCTTCAATAATATTGAAAAAG
GAAGAGTATGAGTATTCAACATTTCCGTGTCGCCCTT
ATTCCCTTTTTTGCGGCATTTTGCCTTCCTGTTTTTGC
TCACCCAGAAACGCTGGTGAAAGTAAAAGATGCTGAA
GATCAGTTGGGTGCACGAGTGGGTTACATCGAACTGGA
TCTCAACAGCGGTAAGATCCTTGAGAGTTTTCGCCCC
GAAGAACGTTTTCCAATGATGAGCACTTTTAAAGTTCT
GCTATGTGGCGCGGTATTATCCCGTGTTGACGCCGGG
CAAGAGCAACTCGGTCGCCGCATACACTATTCTCAGAA
TGACTTGGTTGAGTACTCACCAGTCACAGAAAAGCAT
CTTACGGATGGCATGACAGTAAGAGAATTATGCAGTGC
TGCCATAACCATGAGTGATAACACTGCGGCCAACTTA
CTTCTGACAACGATCGGAGGACCGAAGGAGCTAACCGC
TTTTTTGCACAACATGGGGGATCATGTAACTCGCCTT
GATCGTTGGGAACCGGAGCTGAATGAAGCCATACCAAA
CGACGAGCGTGACACCACGATGCCTGTAGCAATGGCA
ACAACGTTGCGCAAACTATTAACTGGCGAACTACTTAC
TCTAGCTTCCCGGCAACAATTAATAGACTGGATGGAG
GCGGATAAAGTTGCAGGACCACTTCTGCGCTCGGCCCT
TCCGGCTGGCTGGTTTATTGCTGATAAATCTGGAGCC
GGTGAGCGTGGGTCTCGCGGTATCATTGCAGCACTGGG
GCCAGATGGTAAGCCCTCCCGTATCGTAGTTATCTAC
ACGACGGGGAGTCAGGCAACTATGGATGAACGAAATAG
ACAGATCGCTGAGATAGGTGCCTCACTGATTAAGCAT
TGGTAACTGTCAGACCAAGTTTACTCATATATACTTTA
GATTGATTTAAAACTTCATTTTTAATTTAAAAGGATC
TAGGTGAAGATCCTTTTTGATAATCTCATGACCAAAAT
CCCTTAACGTGAGTTTTCGTTCCACTGAGCGTCAGAC
CCCGTAGAAAAGATCAAAGGATCTTCTTGAGATCCTTT
TTTTCTGCGCGTAATCTGCTGCTTGCAAACAAAAAAA
CCACCGCTACCAGCGGTGGTTTGTTTGCCGGATCAAGA
GCTACCAACTCTTTTTCCGAAGGTAACTGGCTTCAGC
AGAGCGCAGATACCAAATACTGTCCTTCTAGTGTAGCC
GTAGTTAGGCCACCACTTCAAGAACTCTGTAGCACCG
CCTACATACCTCGCTCTGCTAATCCTGTTACCAGTGGC
TGCTGCCAGTGGCGATAAGTCGTGTCTTACCGGGTTG
GACTCAAGACGATAGTTACCGGATAAGGCGCAGCGGTC
GGGCTGAACGGGGGGTTCGTGCACACAGCCCAGCTTG
GAGCGAACGACCTACACCGAACTGAGATACCTACAGCG
TGAGCTATGAGAAAGCGCCACGCTTCCCGAAGGGAGA
AAGGCGGACAGGTATCCGGTAAGCGGCAGGGTCGGAAC
AGGAGAGCGCACGAGGGAGCTTCCAGGGGGAAACGCC
TGGTATCTTTATAGTCCTGTCGGGTTTCGCCACCTCTG
ACTTGAGCGTCGATTTTTGTGATGCTCGTCAGGGGGG
CGGAGCCTATGGAAAAACGCCAGCAACGCGGCCTTTTT
ACGGTTCCTGGCCTTTTGCTGGCCTTTTGCTCACATG
TTCTTTCCTGCGTTATCCCCTGATTCTGTGGATAACCG
TATTACCGCCTTTGAGTGAGCTGATACCGCTCGCCGC
AGCCGAACGACCGAGCGCAGCGAGTCAGTGAGCGAGGA
AGCGGAAGAGCGCCTGATGCGGTATTTTCTCCTTACG
CATCTGTGCGGTATTTCACACCGCATATGGTGCACTCT
CAGTACAATCTGCTCTGATGCCGCATAGTTAAGCCAG
TATACACTCCGCTATCGCTACGTGACTGGGTCATGGCT
GCGCCCCGACACCCGCCAACACCCGCTGACGCGCCCT
GACGGGCTTGTCTGCTCCCGGCATCCGCTTACAGACAA
GCTGTGACCGTCTCCGGGAGCTGCATGTGTCAGAGGT
TTTCACCGTCATCACCGAAACGCGCGAGGCAGCAGATC
AATTCGCGCGCGAAGGCGAAGCGGCATGCATTTACGT
TGACACCATCGAATGGTGCAAAACCTTTCGCGGTATGG
CATGATAGCGCCCGGAAGAGAGTCAATTCAGGGTGGT
GAATGTGAAACCAGTAACGTTATACGATGTCGCAGAGT
ATGCCGGTGTCTCTTATCAGACCGTTTCCCGCGTGGT
GAACCAGGCCAGCCACGTTTCTGCGAAAACGCGGGAAA
AAGTGGAAGCGGCGATGGCGGAGCTGAATTACATTCC
CAACCGCGTGGCACAACAACTGGCGGGCAAACAGTCGT
TGCTGATTGGCGTTGCCACCTCCAGTCTGGCCCTGCA
CGCGCCGTCGCAAATTGTCGCGGCGATTAAATCTCGCG
CCGATCAACTGGGTGCCAGCGTGGTGGTGTCGATGGT
AGAACGAAGCGGCGTCGAAGCCTGTAAAGCGGCGGTGC
ACAATCTTCTCGCGCAACGCGTCAGTGGGCTGATCAT
TAACTATCCGCTGGATGACCAGGATGCCATTGCTGTGG
AAGCTGCCTGCACTAATGTTCCGGCGTTATTTCTTGA
TGTCTCTGACCAGACACCCATCAACAGTATTATTTTCT
CCCATGAAGACGGTACGCGACTGGGCGTGGAGCATCT
GGTCGCATTGGGTCACCAGCAAATCGCGCTGTTAGCGG
GCCCATTAAGTTCTGTCTCGGCGCGTCTGCGTCTGGC
TGGCTGGCATAAATATCTCACTCGCAATCAAATTCAGC
CGATAGCGGAACGGGAAGGCGACTGGAGTGCCATGTC
CGGTTTTCAACAAACCATGCAAATGCTGAATGAGGGCA
TCGTTCCCACTGCGATGCTGGTTGCCAACGATCAGAT
GGCGCTGGGCGCAATGCGCGCCATTACCGAGTCCGGGC
TGCGCGTTGGTGCGGATATCTCGGTAGTGGGATACGA
CGATACCGAAGACAGCTCATGTTATATCCCGCCGTCAA
CCACCATCAAACAGGATTTTCGCCTGCTGGGGCAAAC
CAGCGTGGACCGCTTGCTGCAACTCTCTCAGGGCCAGG
CGGTGAAGGGCAATCAGCTGTTGCCCGTCTCACTGGT
GAAAAGAAAAACCACCCTGGCGCCCAATACGCAAACCG
CCTCTCCCCGCGCGTTGGCCGATTCATTAATGCAGCT
GGCACGACAGGTTTCCCGACTGGAAAGCGGGCAGTGAG
CGCAACGCAATTAATGTGAGTTAGCGCGAATTGATCT
G

### Example 2

### Modulation of Blood Pressure by Insulin-Degrading Enzyme Inhibitors

In order to determine whether IDE inhibitors modulate blood pressure, the effect of 6bk in a mouse Calcitonin Gene-Related Peptide (CGRP) model was evaluated. CGRP is a potent microvascular vasodilator widely expressed in the central and peripheral nervous systems of vertebrates. Administration of CGRP resulting in supraphysiological plasma levels of CGRP results in temporary vasodilation, hypotension, and an increased heart rate. This effect has been demonstrated after intravenous administration of CGRP in various vertebrate species, including humans.

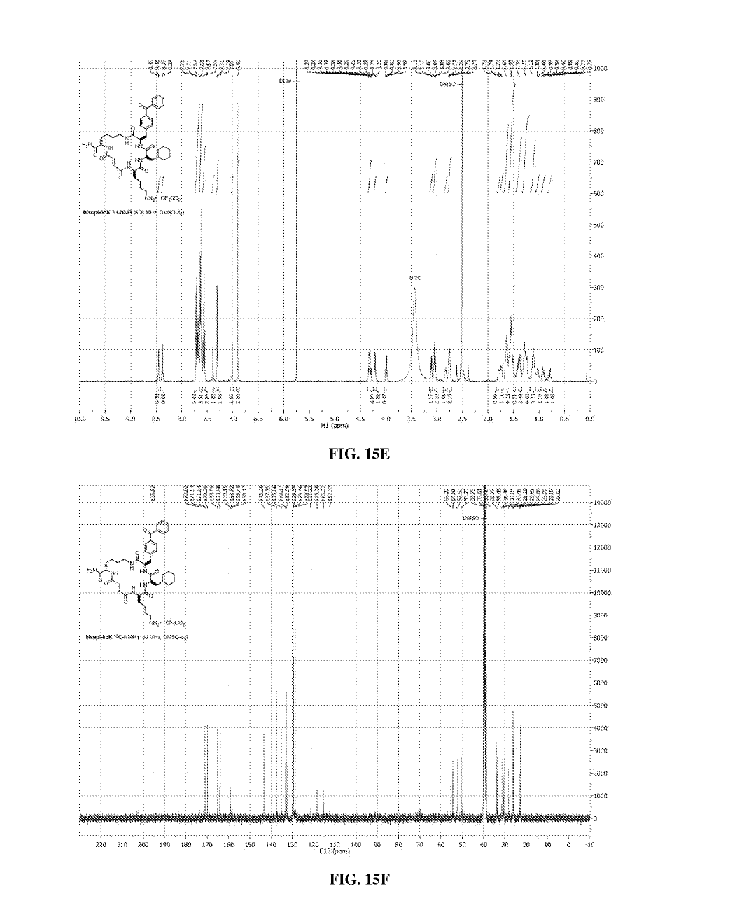

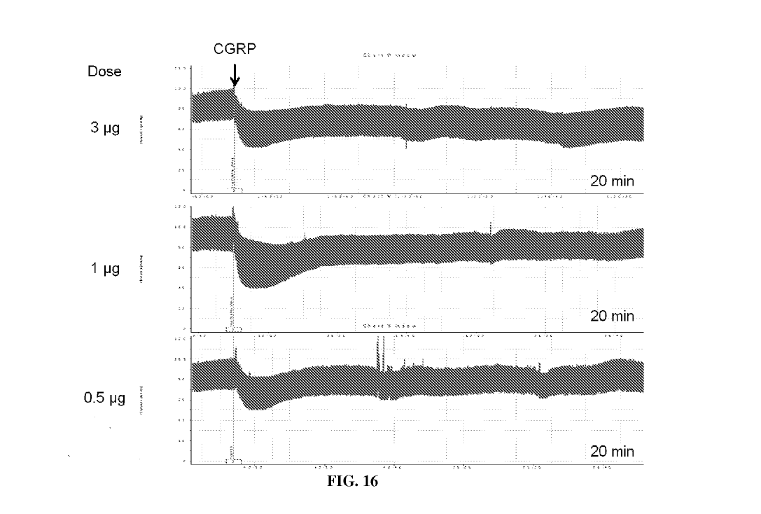

CGRP-induced hypotension is dose-dependent, as illustrated in FIG. 16, showing data from a single mouse injected with different doses (0.5 μg, 1 μg, and 3 μg) of CGRP. Blood pressure was measured over a time of 20 minutes after administration of CGRP. A dose-dependent, temporary induction of hypertension by CGRP was observed.

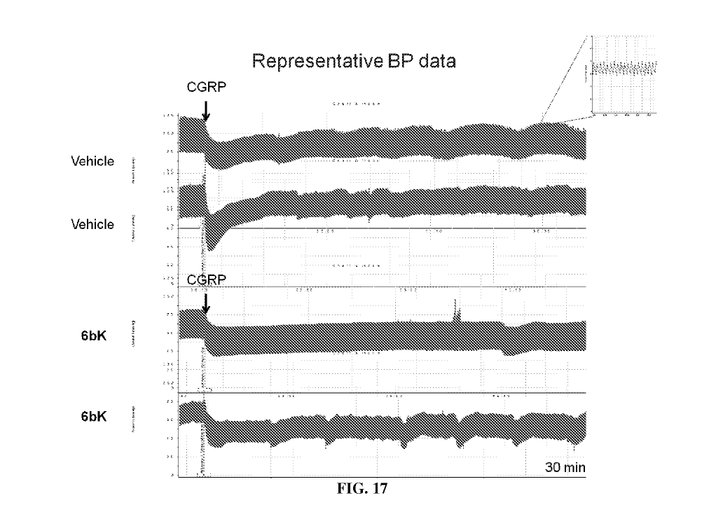

FIG. 17 illustrates representative blood pressure data obtained after administration of 0.25 μg CGRP alone or in combination with 6bK. Blood pressure was monitored for 30 minutes after administration of CGRP.

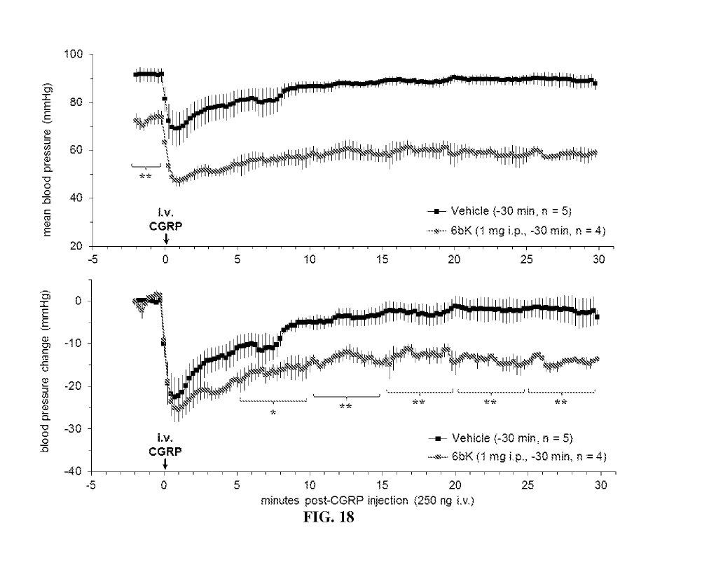

FIG. 18 illustrates that 6bK affects blood pressure baseline and increases CGRP response duration. Nine mice received 250 ng CGRP intravenously (t=0). Four of these mice received 1 mg of 6bK intraperitoneally 30 minutes before CGRP administration. The other five received a control injection of vehicle only. The upper panel of FIG. 18 demonstrates that baseline blood pressure was reduced in 6bK-administered mice as compared to mice that received a control injection of vehicle only. In addition, 6bK attenuated return of blood pressure to baseline levels after CGRP administration. The lower panel of FIG. 18 shows that while the initial decrease in blood pressure levels followed similar kinetics in both 6bK-administered and control mice, the return to baseline followed slower kinetics in 6bK-administered mice as compared to control mice.

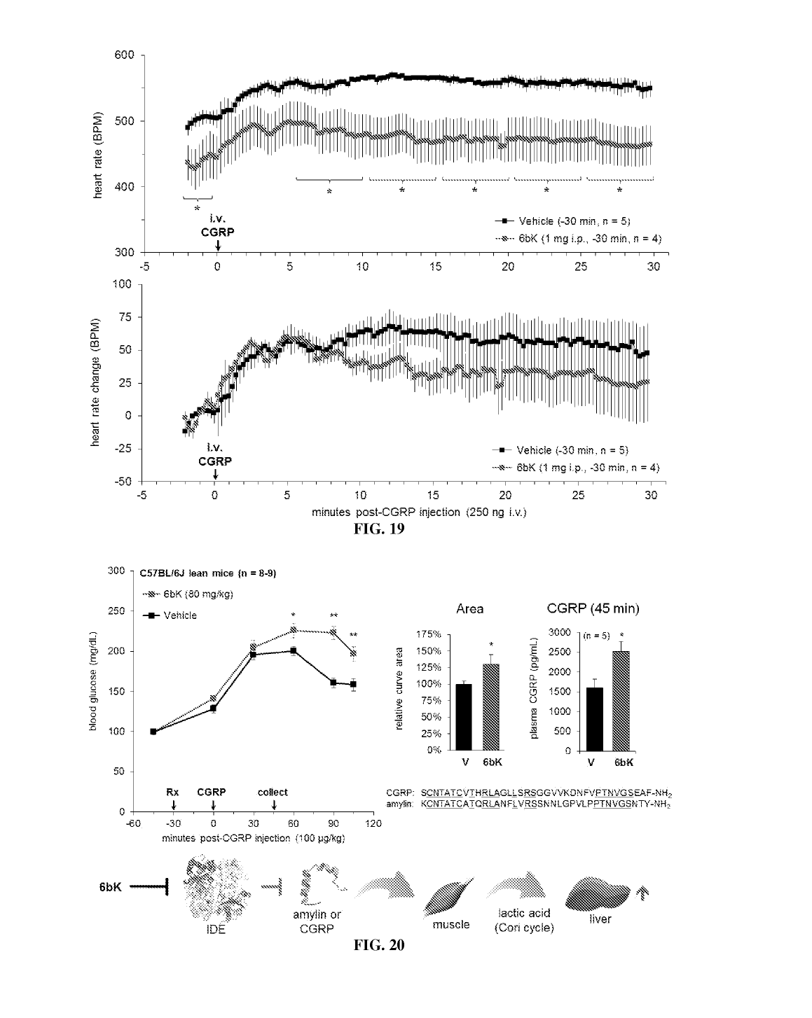

To test whether 6bK affects baseline heart rate and heart rate changes induced by CGRP administration, four mice were injected with 1 mg 6bK 30 min before intravenous administration0 of 250 ng CGRP (t=0). The upper panel of FIG. 19 demonstrates that the baseline heart rate was reduced in 6bK-administered mice as compared to control mice. In addition,the lower panel of FIG. 19 demonstates that the increase in heart rate observed initially after CGRP administration is similar in both groups, but that the heart rate remains lower and the heart change rate is decreased in 6bK-administered mice as compared to controls.

Supraphysiological doses of CGRP induce blood glucose fluctuations via amylin receptors. In order to test whether 6bK modulates these CGRP-induced blood glucoe excursions, C57BL/6J lean mice were injected with either 80 mg/kg 6bK or with vehicle alone 30 min before administration of CGRP. Blood glucose was monitored at various points throughout the experiment, beginning at 45 minutes before and ending at 110 minutes after CGRP administration. The data was plotted (left panel) and the area under the curve for the 6bK-administered mice and the control mice was integrated. Blood was obtained 45 minutes after 6bK administration and plasma CGRP levels were measured. FIG. 20 demonstrates that 6bK augments CGRP-induced blood glucose excursions.

The data indicate that 6bK increases the duration of CGRP-induced hypotension, suggesting that IDE enzymatic processing determines the physiological activity of CGRP in vivo, and might be applicable to pharmacological modulation of the endogenous vasodilator. The observed effect of 6bK on hypotension is corroborated by the observed 6bK augmentation of CGRP-induced blood glucose level fluctuations. In addition, 6bK treatment also lowered baseline blood pressure 10 to 15 min post-injection.

### REFERENCES

1 Drag, M. & Salvesen, G. S. Emerging principles in protease-based drug discovery. Nat Rev Drug Discov 9, 690-701, doi:nrd3053[pii] 10.1038/nrd3053 (2010).
2 Drucker, D. J. The biology of incretin hormones. Cell Metab 3, 153-165, doi:S1550-4131(06)00028-3 [pii] 10.1016/j.cmet.2006.01.004 (2006).
3 Duckworth, W. C., Bennett, R. G. & Hamel, F. G. Insulin degradation: progress and potential. Endocr Rev 19, 608-624 (1998).
4 Mirsky, I. A. & Broh-Kahn, R. H. The inactivation of insulin by tissue extracts; the distribution and properties of insulin inactivating extracts. Arch Biochem 20, 1-9 (1949).
5 Karamohamed, S. et al. Polymorphisms in the insulin-degrading enzyme gene are associated with type 2 diabetes in men from the NHLBI Framingham Heart Study. Diabetes 52, 1562-1567 (2003).
6 Gu, H. F. et al. Quantitative trait loci near the insulin-degrading enzyme (IDE) gene contribute to variation in plasma insulin levels. Diabetes 53, 2137-2142 (2004).
7 Sladek, R. et al. A genome-wide association study identifies novel risk loci for type 2 diabetes. Nature 445, 881-885, doi:10.1038/nature05616 (2007).
8 Zeggini, E. et al. Replication of genome-wide association signals in UK samples reveals risk loci for type 2 diabetes. Science 316, 1336-1341, doi:10.1126/science.1142364 (2007).
9 Bartl, J. et al. Disorder-specific effects of polymorphisms at opposing ends of the Insulin Degrading Enzyme gene. BMC medical genetics 12, 151, doi:10.1186/1471-2350-12-151 (2011).
10 Farris, W. et al. Insulin-degrading enzyme regulates the levels of insulin, amyloid beta-protein, and the beta-amyloid precursor protein intracellular domain in vivo. Proc Natl Acad Sci USA 100, 4162-4167, doi:10.1073/pnas.0230450100 [pii] (2003).
11 Abdul-Hay, S. O. et al. Deletion of insulin-degrading enzyme elicits antipodal, age-dependent effects on glucose and insulin tolerance. PLoS One 6, e20818, doi:10.1371/journal.pone.0020818 PONE-D-10-05674 [pii] (2011).
12 Leissring, M. A. et al. Designed inhibitors of insulin-degrading enzyme regulate the catabolism and activity of insulin. PLoS One 5, e10504, doi:10.1371/journal.pone.0010504 (2010).
13 Knight, Z. A. & Shokat, K. M. Chemical genetics: where genetics and pharmacology meet. Cell 128, 425-430, doi:10.1016/j.cell.2007.01.021 (2007).
14 Workman, P. & Collins, I. Probing the probes: fitness factors for small molecule tools. Chem Biol 17, 561-577, doi:10.1016/j.chembiol.2010.05.013 (2010).
15 Gartner, Z. J. et al. DNA-templated organic synthesis and selection of a library of macrocycles. Science 305, 1601-1605, doi:10.1126/science.1102629 [pii] (2004).
16 Tse, B. N., Snyder, T. M., Shen, Y. & Liu, D. R. Translation of DNA into a library of 13,000 synthetic small-molecule macrocycles suitable for in vitro selection. J Am Chem Soc 130, 15611-15626, doi:10.1021/ja805649f (2008).
17 Kleiner, R. E., Dumelin, C. E., Tiu, G. C., Sakurai, K. & Liu, D. R. In vitro selection of a DNA-templated small-molecule library reveals a class of macrocyclic kinase inhibitors. J Am Chem Soc 132, 11779-11791, doi:10.1021/ja104903x (2010).
18 Kim, Y. G., Lone, A. M., Nolte, W. M. & Saghatelian, A. Peptidomics approach to elucidate the proteolytic regulation of bioactive peptides. Proc Natl Acad Sci USA 109, 8523-8527, doi:10.1073/pnas.1203195109 (2012).
19 Saghatelian, A., Jessani, N., Joseph, A., Humphrey, M. & Cravatt, B. F. Activity-based probes for the proteomic profiling of metalloproteases. Proc Natl Acad Sci USA 101, 10000-10005, doi:10.1073/pnas.0402784101 (2004).
20 Malito, E. et al. Molecular Bases for the Recognition of Short Peptide Substrates and Cysteine-Directed Modifications of Human Insulin-Degrading Enzymet. Biochemistry 47, 12822-12834, doi:10.1021/bi801192h (2008).
21 Malito, E., Hulse, R. E. & Tang, W. J. Amyloid beta-degrading cryptidases: insulin degrading enzyme, presequence peptidase, and neprilysin. Cellular and molecular life sciences: CMLS 65, 2574-2585, doi:10.1007/s00018-008-8112-4 (2008).
22 Shen, Y., Joachimiak, A., Rosner, M. R. & Tang, W. J. Structures of human insulin-degrading enzyme reveal a new substrate recognition mechanism. Nature 443, 870-874, doi:10.1038/nature05143 (2006).
23 Guo, Q., Manolopoulou, M., Bian, Y., Schilling, A. B. & Tang, W. J. Molecular basis for the recognition and cleavages of IGF-II, TGF-alpha, and amylin by human insulin-degrading enzyme. J Mol Biol 395, 430-443, doi:10.1016/j.jmb.2009.10.072 (2010).
24 Manolopoulou, M., Guo, Q., Malito, E., Schilling, A. B. & Tang, W. J. Molecular basis of catalytic chamber-assisted unfolding and cleavage of human insulin by human insulin-degrading enzyme. J Biol Chem 284, 14177-14188, doi:10.1074/jbc.M900068200 (2009).
25 Stella, V. J. & He, Q. Cyclodextrins. Toxicologic pathology 36, 30-42, doi:10.1177/0192623307310945 (2008).
26 Andrikopoulos, S., Blair, A. R., Deluca, N., Fam, B. C. & Proietto, J. Evaluating the glucose tolerance test in mice. American journal of physiology. Endocrinology and metabolism 295, E1323-1332, doi:10.1152/ajpendo.90617.2008 (2008).
27 Monzillo, L. U. & Hamdy, O. Evaluation of insulin sensitivity in clinical practice and in research settings. Nutrition reviews 61, 397-412 (2003).
28 Mirsky, I. A. & Perisutti, G. Effect of insulinase-inhibitor on hypoglycemic action of insulin. Science 122, 559-560 (1955).
29 Ahren, B., Winzell, M. S. & Pacini, G. The augmenting effect on insulin secretion by oral versus intravenous glucose is exaggerated by high-fat diet in mice. The Journal of endocrinology 197, 181-187, doi:10.1677/JOE-07-0460 (2008).
30 Winzell, M. S. & Ahren, B. The high-fat diet-fed mouse: a model for studying mechanisms and treatment of impaired glucose tolerance and type 2 diabetes. Diabetes 53 Suppl 3, S215-219 (2004).
31 Riddle, M. C. & Drucker, D. J. Emerging therapies mimicking the effects of amylin and glucagon-like peptide 1. Diabetes Care 29, 435-449 (2006).
32 Mooradian, A. D. & Thurman, J. E. Drug therapy of postprandial hyperglycaemia. Drugs 57, 19-29 (1999).
33 Malaisse, W. J. Pharmacology of the meglitinide analogs: new treatment options for type 2 diabetes mellitus. Treatments in endocrinology 2, 401-414 (2003).
34 Hollander, P. et al. Effect of pramlintide on weight in overweight and obese insulin-treated type 2 diabetes patients. Obesity research 12, 661-668, doi:10.1038/oby.2004.76 (2004).
35 Duckworth, W. C. & Kitabchi, A. E. Insulin and glucagon degradation by the same enzyme. Diabetes 23, 536-543 (1974).
36 Shroyer, L. A. & Varandani, P. T. Purification and characterization of a rat liver cytosol neutral thiol peptidase that degrades glucagon, insulin, and isolated insulin A and B chains. Archives of biochemistry and biophysics 236, 205-219 (1985).
37 Bennett, R. G., Duckworth, W. C. & Hamel, F. G. Degradation of amylin by insulin-degrading enzyme. J Biol Chem 275, 36621-36625, doi:10.1074/jbc.M006170200 (2000).
38 Authier, F., Mort, J. S., Bell, A. W., Posner, B. I. & Bergeron, J. J. Proteolysis of glucagon within hepatic endosomes by membrane-associated cathepsins B and D. J Biol Chem 270, 15798-15807 (1995).
39 Fontes, G. et al. Miniglucagon (MG)-generating endopeptidase, which processes glucagon into MG, is composed of N-arginine dibasic convertase and aminopeptidase B. Endocrinology 146, 702-712, doi:10.1210/en.2004-0853 (2005).
40 Trebbien, R. et al. Neutral endopeptidase 24.11 is important for the degradation of both endogenous and exogenous glucagon in anesthetized pigs. American journal of physiology. Endocrinology and metabolism 287, E431-438, doi:10.1152/ajpendo.00353.2003 (2004).
41 Young, A. Effects on plasma glucose and lactate. Adv Pharmacol 52, 193-208, doi:10.1016/S1054-3589(05)52010-6 (2005).
42 Gelling, R. W. et al. Lower blood glucose, hyperglucagonemia, and pancreatic alpha cell hyperplasia in glucagon receptor knockout mice. Proc Natl Acad Sci USA 100, 1438-1443, doi:10.1073/pnas.0237106100 (2003).
43 Kolterman, O. G., Gottlieb, A., Moyses, C. & Colburn, W. Reduction of postprandial hyperglycemia in subjects with IDDM by intravenous infusion of AC137, a human amylin analogue. Diabetes Care 18, 1179-1182 (1995).
44 Riediger, T., Zuend, D., Becskei, C. & Lutz, T. A. The anorectic hormone amylin contributes to feeding-related changes of neuronal activity in key structures of the gut-brain axis. American journal of physiology. Regulatory, integrative and comparative physiology 286, R114-122, doi:10.1152/ajpregu.00333.2003 (2004).
45 Young, A. Inhibition of glucagon secretion. Adv Pharmacol 52, 151-171, doi:10.1016/S1054-3589(05)52008-8 (2005).
46 Schmitz, O., Brock, B. & Rungby, J. Amylin agonists: a novel approach in the treatment of diabetes. Diabetes 53 Suppl 3, S233-238 (2004).
47 Crowell, M. D., Mathis, C., Schettler, V. A., Yunus, T. & Lacy, B. E. The effects of tegaserod, a 5-HT receptor agonist, on gastric emptying in a murine model of diabetes mellitus. Neurogastroenterology and motility: the official journal of the European Gastrointestinal Motility Society 17, 738-743, doi:10.1111/j.1365-2982.2005.00681.x (2005).
48 Gedulin, B. R., Jodka, C. M., Herrmann, K. & Young, A. A. Role of endogenous amylin in glucagon secretion and gastric emptying in rats demonstrated with the selective antagonist, AC187. Regulatory peptides 137, 121-127, doi:10.1016/j.regpep.2006.06.004 (2006).
49 Unger, R. H. & Cherrington, A. D. Glucagonocentric restructuring of diabetes: a pathophysiologic and therapeutic makeover. The Journal of clinical investigation 122, 4-12, doi:10.1172/JC160016 (2012).
50 Sadry, S. A. & Drucker, D. J. Emerging combinatorial hormone therapies for the treatment of obesity and T2DM. Nature reviews. Endocrinology, doi:10.1038/nrendo.2013.47 (2013).
51 Kleiner, R. E., Dumelin, C. E., Tiu, G. C., Sakurai, K. & Liu, D. R. In vitro selection of a DNA-templated small-molecule library reveals a class of macrocyclic kinase inhibitors. J Am Chem Soc 132, 11779-11791, doi:10.1021/ja104903x (2010).
52 Gartner, Z. J. et al. DNA-templated organic synthesis and selection of a library of macrocycles. Science 305, 1601-1605, doi:10.1126/science.1102629 [pii] (2004).
53 Tse, B. N., Snyder, T. M., Shen, Y. & Liu, D. R. Translation of DNA into a library of 13,000 synthetic small-molecule macrocycles suitable for in vitro selection. J Am Chem Soc 130, 15611-15626, doi:10.1021/ja805649f (2008).
54 Parker, M. G. & Weitzman, P. D. The regulation of Acinetobacter sp. alpha-oxoglutarate dehydrogenase complex. Biochem J 130, 39P (1972).
55 Schenker, P. & Baici, A. Simultaneous interaction of enzymes with two modifiers: reappraisal of kinetic models and new paradigms. J Theor Biol 261, 318-329, doi:10.1016/j.jtbi.2009.07.033 (2009).
56 Kim, Y. G., Lone, A. M., Nolte, W. M. & Saghatelian, A. Peptidomics approach to elucidate the proteolytic regulation of bioactive peptides. Proc Natl Acad Sci USA 109, 8523-8527, doi:10.1073/pnas.1203195109 (2012).
57 Gottlieb, H. E., Kotlyar, V. & Nudelman, A. NMR Chemical Shifts of Common Laboratory Solvents as Trace Impurities. The Journal of organic chemistry 62, 7512-7515 (1997).
58 Georghiou, G., Kleiner, R. E., Pulkoski-Gross, M., Liu, D. R. & Seeliger, M. A. Development and structure-based mechanism of highly specific macrocyclic Src kinase inhibitors from a DNA-template library. submitted (2011).
59 Stella, V. J. & He, Q. Cyclodextrins. Toxicologic pathology 36, 30-42, doi:10.1177/0192623307310945 (2008).
60 Shen, Y., Joachimiak, A., Rosner, M. R. & Tang, W. J. Structures of human insulin-degrading enzyme reveal a new substrate recognition mechanism. Nature 443, 870-874, doi:10.1038/nature05143 (2006).
61 Vonrhein, C. et al. Data processing and analysis with the autoPROC toolbox. Acta crystallographica. Section D, Biological crystallography 67, 293-302, doi:10.1107/50907444911007773 (2011).
62 McCoy, A. J. et al. Phaser crystallographic software. Journal of applied crystallography 40, 658-674, doi:10.1107/s0021889807021206 (2007).
63 Emsley, P. & Cowtan, K. Coot: model-building tools for molecular graphics. Acta crystallographica. Section D, Biological crystallography 60, 2126-2132, doi:10.1107/S0907444904019158 (2004).
64 Adams, P. D. et al. PHENIX: a comprehensive Python-based system for macromolecular structure solution. Acta Crystallographica Section D 66, 213-221, doi:doi:10.1107/S0907444909052925 (2010).
65 Lang, P. T. et al. DOCK 6: combining techniques to model RNA-small molecule complexes. Rna 15, 1219-1230, doi:10.1261/rna.1563609 (2009).
66 Moustakas, D. T. et al. Development and validation of a modular, extensible docking program: DOCK 5. Journal of computer-aided molecular design 20, 601-619, doi:10.1007/s10822-006-9060-4 (2006).
67 Andrikopoulos, S., Blair, A. R., Deluca, N., Fam, B. C. & Proietto, J. Evaluating the glucose tolerance test in mice. American journal of physiology. Endocrinology and metabolism 295, E1323-1332, doi:10.1152/ajpendo.90617.2008 (2008).
68 Llovera, R. E. et al. The catalytic domain of insulin-degrading enzyme forms a denaturant-resistant complex with amyloid beta peptide: implications for Alzheimer disease pathogenesis. J Biol Chem 283, 17039-17048, doi:10.1074/jbc.M706316200 (2008).
69 Lee, Y. et al. Metabolic manifestations of insulin deficiency do not occur without glucagon action. Proc Natl Acad Sci USA 109, 14972-14976, doi:10.1073/pnas.1205983109 (2012).
70 Mirsky, I. A. & Broh-Kahn, R. H. The inactivation of insulin by tissue extracts; the distribution and properties of insulin inactivating extracts. Arch Biochem 20, 1-9 (1949).
71 Duckworth, W. C., Bennett, R. G. & Hamel, F. G. Insulin degradation: progress and potential. Endocr Rev 19, 608-624 (1998).
72 Farris, W. et al. Insulin-degrading enzyme regulates the levels of insulin, amyloid beta-protein, and the beta-amyloid precursor protein intracellular domain in vivo. Proc Natl Acad Sci USA 100, 4162-4167, doi:10.1073/pnas.0230450100 [pii] (2003).
73 Abdul-Hay, S. O. et al. Deletion of insulin-degrading enzyme elicits antipodal, age-dependent effects on glucose and insulin tolerance. PLoS One 6, e20818, doi:10.1371/journal.pone.0020818PONE-D-10-05674 [pii] (2011).
74 Kurochkin, I. V. & Goto, S. Alzheimer's beta-amyloid peptide specifically interacts with and is degraded by insulin degrading enzyme. FEBS letters 345, 33-37 (1994).
75 Qiu, W. Q. et al. Insulin-degrading enzyme regulates extracellular levels of amyloid beta-protein by degradation. J Biol Chem 273, 32730-32738 (1998).
76 Malito, E., Hulse, R. E. & Tang, W. J. Amyloid beta-degrading cryptidases: insulin degrading enzyme, presequence peptidase, and neprilysin. Cellular and molecular life sciences : CMLS 65, 2574-2585, doi:10.1007/s00018-008-8112-4 (2008).
77 Miller, B. C. et al. Amyloid-beta peptide levels in brain are inversely correlated with insulysin activity levels in vivo. Proc Natl Acad Sci USA 100, 6221-6226, doi:10.1073/pnas.1031520100 (2003).
78 Duckworth, W. C. & Kitabchi, A. E. Insulin and glucagon degradation by the same enzyme. Diabetes 23, 536-543 (1974).
79 Shroyer, L. A. & Varandani, P. T. Purification and characterization of a rat liver cytosol neutral thiol peptidase that degrades glucagon, insulin, and isolated insulin A and B chains. Archives of biochemistry and biophysics 236, 205-219 (1985).
80 Bennett, R. G., Duckworth, W. C. & Hamel, F. G. Degradation of amylin by insulin-degrading enzyme. J Biol Chem 275, 36621-36625, doi:10.1074/jbc.M006170200 (2000).
81 Hamel, F. G., Gehm, B. D., Rosner, M. R. & Duckworth, W. C. Identification of the cleavage sites of transforming growth factor alpha by insulin-degrading enzymes. Biochimica et biophysica acta 1338, 207-214 (1997).
82 Misbin, R. I., Almira, E. C., Duckworth, W. C. & Mehl, T. D. Inhibition of insulin degradation by insulin-like growth factors. Endocrinology 113, 1525-1527 (1983).
83 Ciaccio, C. et al. Somatostatin: a novel substrate and a modulator of insulin-degrading enzyme activity. J Mol Biol 385, 1556-1567, doi:10.1016/j.jmb.2008.11.025 (2009).
84 Safavi, A., Miller, B. C., Cottam, L. & Hersh, L. B. Identification of gamma-endorphin-generating enzyme as insulin-degrading enzyme. Biochemistry 35, 14318-14325, doi:10.1021/bi960582q (1996).
85 Muller, D., Baumeister, H., Buck, F. & Richter, D. Atrial natriuretic peptide (ANP) is a high-affinity substrate for rat insulin-degrading enzyme. European journal of biochemistry / FEBS 202, 285-292 (1991).
86 Muller, D., Schulze, C., Baumeister, H., Buck, F. & Richter, D. Rat insulin-degrading enzyme: cleavage pattern of the natriuretic peptide hormones ANP, BNP, and CNP revealed by HPLC and mass spectrometry. Biochemistry 31, 11138-11143 (1992).
87 Bennett, R. G., Heimann, D. G. & Hamel, F. G. Degradation of relaxin family peptides by insulin-degrading enzyme. Annals of the New York Academy of Sciences 1160, 38-41, doi:10.1111/j.1749-6632.2008.03782.x (2009).
88 Zhang, W. J., Luo, X. & Guo, Z. Y. In vitro degradation of insulin-like peptide 3 by insulin-degrading enzyme. The protein journal 29, 93-98, doi:10.1007/s10930-009-9226-8 (2010).

All publications, patents, patent applications, publication, and database entries (e.g., sequence database entries) mentioned herein, e.g., in the Background, Summary, Derailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### Equivalents and Scope

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

1. A method for identifying insulin-degrading enzyme (IDE)-binding compounds, the method comprising
(a) contacting an IDE with
(i) a probe that binds IDE with an IC₅₀ of 10 μM or less, wherein the probe comprises a detectable label; and
(ii) a candidate compound,
under conditions suitable for the probe and the candidate compound to bind the IDE;
(b) determining the level of unbound probe in the presence of the candidate compound; and
(c) comparing the level of unbound probe determined in step (b) to a reference level, wherein if the level of unbound probe in the presence of the candidate compound is higher than the reference level, then the candidate compound is identified as an IDE-binding compound.

2. The method of claim 1, wherein step (a) comprises contacting the IDE with the probe of (i) before contacting the IDE with the candidate compound.

3. The method of claim 1, wherein the IDE is a catalytically inactive IDE.

4-6. (canceled)

7. The method of claim 1, wherein the IDE-binding probe is a compound of any of Formula (I)-(VI).

8. The method of claim 7, wherein the IDE-binding probe comprises a structure selected from the group consisting of structures 1b, 2b, 3b, 4b, 5b, 6a, 6c, 6b, 6bk, and 1-29.

9. The method of claim 8, wherein the IDE-binding probe comprises structure 6bK.

10. The method of claim 7, wherein the IDE-binding probe is conjugated to the detectable label.

11. (canceled)

12. The method of claim 1, wherein the detectable label comprises a fluorophore.

13-15. (canceled)

16. The method of claim 1, wherein the probe comprises compound 31:

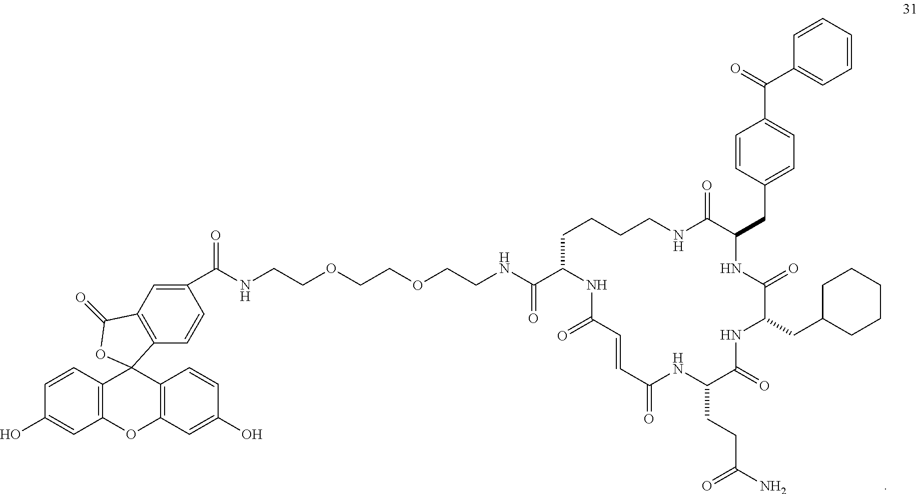 [SMILES: NC(=O)CC[C@@H]1NC(=O)/C=C/C(=O)N[C@H](C(=O)NCCOCCOCCNC(=O)c2ccc3c(c2)C(=O)OC32c3ccc(O)cc3Oc3cc(O)ccc32)CCCCNC(=O)[C@@H](Cc2ccc(C(=O)c3ccccc3)cc2)NC(=O)[C@H](CC2CCCCC2)NC1=O]

17. The method of claim 12, wherein step (b) comprises
(i) exposing the IDE molecule contacted with the probe and the candidate compound of step (a) to polarized light of a suitable wave length to excite the fluorophore; and
(ii) detecting
(A) the level of fluorescent light emitted by the fluorophore in the same plane of polarization as the incident light, and
(B) the level of fluorescent light emitted by the fluorophore in a plane different from the plane of polarization of the incident light.

18. The method of claim 17, wherein step (b) comprises calculating the level of unbound probe from the levels of emitted light detected in (ii)(A) and (ii)(B).

19-24. (canceled)

25. The method of claim 1, wherein the detectable label comprises a binding agent.

26-29. (canceled)

30. The method of claim 1, wherein the detectable label comprises a detectable isotope.

31-33. (canceled)

34. The method of claim 1, wherein the method comprises screening a library of different candidate compounds.

35. (canceled)

36. The method of claim 1, wherein the method comprises performing steps (a)-(c) in parallel for a plurality of different candidate compounds.

37. (canceled)

38. The method of claim 1, wherein the reference level represents a level of unbound probe in the absence of the candidate compound.

39. The method of claim 1, wherein the reference level is determined by measuring the level of unbound probe in the absence of a candidate compound or in the presence of a compound known to bind IDE with an IC₅₀ of greater than approximately 10 μM.

40. The method of claim 1, wherein the probe is an IDE inhibitor and the method further comprises identifying the IDE-binding compound as an IDE-inhibitor.

41. (canceled)

42. A compound of Formula (V): 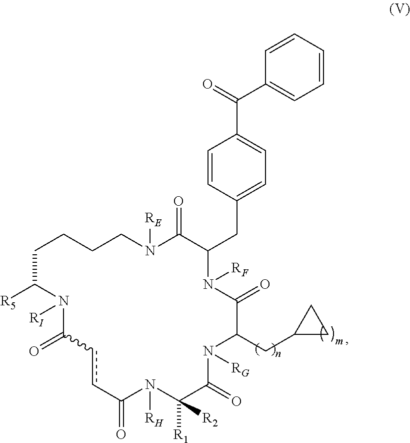
or a pharmaceutically acceptable salt, solvate, hydrate, stereoisomer, polymorph, tautomer, isotopically enriched form, or prodrug thereof,
wherein [symbol]
is a single or double C—C bond, wherein when [symbol] is a double C—C bond, then [symbol] indicates that the adjacent C—C double bond is in a cis or trans configuration;
R₁ is hydrogen; halogen; substituted or unsubstituted aliphatic; substituted or unsubstituted heteroaliphatic; substituted or unsubstituted aryl; substituted or unsubstituted heteroaryl; substituted or unsubstituted acyl; —OR_{A}; —N(R_{A})₂; —SR_{A}; ═O; —CN; —NO₂; —SCN; —SOR_{A}; or —SO₂R_{A}; wherein each occurrence of R_{A} is independently hydrogen; a protecting group; substituted or unsubstituted aliphatic; substituted or unsubstituted heteroaliphatic; substituted or unsubstituted acyl; substituted or unsubstituted aryl; or substituted or unsubstituted heteroaryl;
R₂ is hydrogen; halogen; substituted or unsubstituted aliphatic; substituted or unsubstituted heteroaliphatic; substituted or unsubstituted aryl; substituted or unsubstituted heteroaryl; substituted or unsubstituted acyl; —OR_{B}; —N(R_{B})₂; —SR_{B}; ═O; —CN; —NO₂; —SCN; —SOR_{B}; or SO₂R_{B}; wherein each occurrence of R_{B} independently hydrogen; a protecting group; substituted or unsubstituted aliphatic; substituted or unsubstituted heteroaliphatic; substituted or unsubstituted acyl; substituted or unsubstituted aryl; or substituted or unsubstituted heteroaryl;
R₅ comprises a detectable label and, optionally, a linker;
each instance of R_{E}, R_{F}, R_{G}, R_{H}, and R_{I} is independently hydrogen; substituted or unsubstituted acyl; a nitrogen protecting group; substituted or unsubstituted aliphatic; substituted or unsubstituted heteroaliphatic; substituted or unsubstituted aryl; substituted or unsubstituted heteroaryl; substitute or unsubstituted hydroxyl; substituted or unsubstituted thiol; substituted or unsubstituted amino; or halogen;
n is 0 or an integer between 1 and 10, inclusive; and
m is an integer between 1 and 5, inclusive.

43-73. (canceled)

74. A method comprising administering an IDE inhibitor to a subject in an amount effective to
(a) inhibit IDE activity in the subject;
(b) modulate the stability and/or signaling of amylin in the subject;
(c) modulate the stability and/or signaling of Calcitonin Gene-Related Peptide (CGRP) in the subject; and/or
(d) modulate the stability and/or signaling of glucagon in the subject;
wherein the IDE inhibitor is administered according to a dosing schedule resulting in transient IDE inhibition.

75-109. (canceled)


SEQUENCE LISTING (70 sequences)

SEQ ID NO: 1 - protein, 1019 aa, Homo sapiens [sequence omitted]

SEQ ID NO: 2 - protein, 464 aa, Homo sapiens
MSKLWFKQDDKFFLPKACLNFEFFSPFAYVDPLHCNMAYLYLELLKDSLNEYAYAAELAGLSYDLQNTIYGMYLSVKGYNDKQPILLKKIIEKMATFEIDEKRFEIIKEAYMRSLNNFRAEQPHQHAMYYLRLLMTEVAWTKDELKEALDDVTLPRLKAFIPQLLSRLHIEALLHGNITKQAALGIMQMVEDTLIEHAHTKPLLPSQLVRYREVQLPDRGWFVYQQRNEVHNNCGIEIYYQTDMQSTSENMFLELFCQIISEPCFNTLRTKEQLGYIVFSGPRRANGIQGLRFIIQSEKPPHYLESRVEAFLITMEKSIEDMTEEAFQKHIQALAIRRLDKPKKLSAECAKYWGEIISQQYNFDRDNTEVAYLKTLTKEDIIKFYKEMLAVDAPRRHKVSVHVLAREMDSCPVVGEFPCQNDINLSQAPALPQPEVIQNMTEFKRGLPLFPLVKPHINFMAAKL

SEQ ID NO: 3 - protein, 1019 aa, Mus musculus
MRNGLVWLLHPALPGTLRSILGARPPPAKRLCGFPKQTYSTMSNPAIQRIEDQIVKSPEDKREYRGLELANGIKVLLISDPTTDKSSAALDVHIGSLSDPPNIPGLSHFCEHMLFLGTKKYPKENEYSQFLSEHAGSSNAFTSGEHTNYYFDVSHEHLEGALDRFAQFFLCPLFDASCKDREVNAVDSEHEKNVMNDAWRLFQLEKATGNPKHPFSKFGTGNKYTLETRPNQEGIDVREELLKFHSTYYSSNLMAICVLGRESLDDLTNLVVKLFSEVENKNVPLPEFPEHPFQEEHLRQLYKIVPIKDIRNLYVTFPIPDLQQYYKSNPGHYLGHLIGHEGPGSLLSELKSKGWVNTLVGGQKEGARGFMFFIINVDLTEEGLLHVEDIILHMFQYIQKLRAEGPQEWVFQECKDLNAVAFRFKDKERPRGYTSKIAGKLHYYPLNGVLTAEYLLEEFRPDLIDMVLDKLRPENVRVAIVSKSFEGKTDRTEQWYGTQYKQEAIPEDIIQKWQNADLNGKFKLPTKNEFIPTNFEILSLEKDATPYPALIKDTAMSKLWFKQDDKFFLPKACLNFEFFSPFAYVDPLHCNMAYLYLELLKDSLNEYAYAAELAGLSYDLQNTIYGMYLSVKGYNDKQPILLKKITEKMATFEIDKKRFEIIKEAYMRSLNNFRAEQPHQHAMYYLRLLMTEVAWTKDELKEALDDVTLPRLKAFIPQLLSRLHIEALLHGNITKQAALGVMQMVEDTLIEHAHTKPLLPSQLVRYREVQLPDRGWFVYQQRNEVHNNCGIEIYYQTDMQSTSENMFLELFCQIISEPCFNTLRTKEQLGYIVFSGPRRANGIQGLRFIIQSEKPPHYLESRVEAFLITMEKAIEDMTEEAFQKHIQALAIRRLDKPKKLSAECAKYWGEIISQQYNYDRDNIEVAYLKTLTKDDIIRFYQEMLAVDAPRRHKVSVHVLAREMDSCPVVGEFPSQNDINLSEAPPLPQPEVIHNMTEFKRGLPLFPLVKPHINFMAAKL

SEQ ID NO: 4 - DNA, 7351 bp, Artificial Sequence
  Synthetic Polynucleotide
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggggggttctcatcatcatcatcatcatatgaataatccagccatcaagagaataggaaatcacattaccaagtctcctgaagacaagcgagaatatcgagggctagagctggccaatggtatcaaagtacttcttatcagtgatcccaccacggataagtcatcagcagcacttgatgtgcacataggttcattgtcggatcctccaaatattgctggcttaagtcatttttgtgaacatatgctttttttgggaacaaagaaataccctaaagaaaatgaatacagccagtttctcagtgagcatgcaggaagttcaaatgcctttactagtggagagcataccaattactattttgatgtttctcatgaacacctagaaggtgccctagacaggtttgcacagttttttctgtgccccttgttcgatgaaagttgcaaagacagagaggtgaatgcagttgattcagaacatgagaagaatgtgatgaatgatgcctggagactctttcaattggaaaaagctacagggaatcctaaacaccccttcagtaaatttgggacaggtaacaaatatactctggagactagaccaaaccaagaaggcattgatgtaagacaagagctactgaaattccattctgcttactattcatccaacttaatggctgtttgtgttttaggtcgagaatctttagatgacttgactaatctggtggtaaagttattttctgaagtagagaacaaaaatgttccattgccagaatttcctgaacaccctttccaagaagaacatcttaaacaactttacaaaatagtacccattaaagatattaggaatctctatgtgacatttcccatacctgaccttcagaaatactacaaatcaaatcctggtcattatcttggtcatctcattgggcatgaaggtcctggaagtctgttatcagaacttaagtcaaagggctgggttaatactcttgttggtgggcagaaggaaggagcccgaggttttatgttttttatcattaatgtggacttgaccgaggaaggattattacatgttgaagatataattttgcacatgtttcaatacattcagaagttacgtgcagaaggacctcaagaatgggttttccaagagtgcaaggacttgaatgctgttgcttttaggtttaaagacaaagagaggccacggggctatacatctaagattgcaggaatattgcattattatcccctagaagaggtgctcacagcggaatatttactggaagaatttagacctgacttaatagagatggttctcgataaactcagaccagaaaatgtccgggttgccatagtttctaaatcttttgaaggaaaaactgatcgcacagaagagtggtatggaacccagtacaaacaagaagctataccggatgaagtcatcaagaaatggcaaaatgctgacctgaatgggaaatttaaacttcctacaaagaatgaatttattcctacgaattttgagattttaccgttagaaaaagaggcgacaccataccctgctcttattaaggatacagctatgagcaaactttggttcaaacaagatgataagttttttttgccgaaggcttgtctcaactttgaatttttcagcccatttgcttatgtggaccccttgcactgtaacatggcctatttgtaccttgagctcctcaaagactcactcaacgagtatgcatatgcagcagagctagcaggcttgagctatgatctccaaaataccatctatgggatgtatctttcagtgaaaggttacaatgacaagcagccaattttactaaagaagattattgagaaaatggctacctttgagattgatgaaaaaagatttgaaattatcaaagaagcatatatgcgatctcttaacaatttccgggctgaacagcctcaccagcatgccatgtactacctccgcttgctgatgactgaagtggcctggactaaagatgagttaaaagaagctctggatgatgtaacccttcctcgccttaaggccttcatacctcagctcctgtcacggctgcacattgaagcccttctccatggaaacataacaaagcaggctgcattaggaattatgcagatggttgaagacaccctcattgaacatgctcataccaaacctctccttccaagtcagctggttcggtatagagaagttcagctccctgacagaggatggtttgtttatcagcagagaaatgaagttcacaataactgtggcatcgagatatactaccaaacagacatgcaaagcacctcagagaatatgtttctggagctcttctgtcagattatctcggaaccttgcttcaacaccctgcgcaccaaggagcagttgggctatatcgtcttcagcgggccacgtcgagctaatggcatacagggcttgagattcatcatccagtcagaaaagccacctcactacctagaaagcagagtggaagctttcttaattaccatggaaaagtccatagaggacatgacagaagaggccttccaaaaacacattcaggcattagcaattcgtcgactagacaaaccaaagaagctatctgctgagtgtgctaaatactggggagaaatcatctcccagcaatataattttgacagagataacactgaggttgcatatttaaagacacttaccaaggaagatatcatcaaattctacaaggaaatgttggcagtagatgctccaaggagacataaggtatccgtccatgttcttgccagggaaatggattcttgtcctgttgttggagagttcccatgtcaaaatgacataaatttgtcacaagcaccagccttgccacaacctgaagtgattcagaacatgaccgaattcaagcgtggtctgccactgtttccccttgtgaaaccacatattaacttcatggctgcaaaactctgaggtatggctagcatgactggtggacagcaaatgggtcgggatctgtacgacgatgacgataaggatcgatggggatccgagctcgagatctgcagctggtaccatatgggaattcgaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 5 - protein, 9 aa, Artificial Sequence
  Synthetic Polypeptide
RPPGFSAFK

SEQ ID NO: 6 - protein, 37 aa, Artificial Sequence
  Synthetic Polypeptide
SCNTATCVTHRLAGLLSRSGGVVKDNFVPTNVGSEAF

SEQ ID NO: 7 - protein, 37 aa, Artificial Sequence
  Synthetic Polypeptide
KCNTATCATQRLANFLVRSSNNLGPVLPPTNVGSNTY

SEQ ID NO: 8 - DNA, 37 bp, Artificial Sequence
  Synthetic Polynucleotide
atcatcatatgaataatccagccancaagagaatagg

SEQ ID NO: 9 - DNA, 36 bp, Artificial Sequence
  Synthetic Polynucleotide
atgctagccatacctcagagntttgcagccatgaag

SEQ ID NO: 10 - DNA, 42 bp, Artificial Sequence
  Synthetic Polynucleotide
atggctggattattcatatgatgangatgatgatgagaaccc

SEQ ID NO: 11 - DNA, 29 bp, Artificial Sequence
  Synthetic Polynucleotide
actctgaggtatggctagcangactggtg

SEQ ID NO: 12 - DNA, 35 bp, Artificial Sequence
  Synthetic Polynucleotide
acatgagaagaatgtgatgaatgancagtggagac

SEQ ID NO: 13 - DNA, 29 bp, Artificial Sequence
  Synthetic Polynucleotide
atcattcatcacattcttctcatgntctg

SEQ ID NO: 14 - DNA, 30 bp, Artificial Sequence
  Synthetic Polynucleotide
agactctttcaattggaaaaagcnacaggg

SEQ ID NO: 15 - DNA, 57 bp, Artificial Sequence
  Synthetic Polynucleotide
agctttttccaattgaaagagtcnccaggtatcattcatcacattcttctcatgttc

SEQ ID NO: 16 - DNA, 30 bp, Artificial Sequence; identical to SEQ ID NO: 14
  Synthetic Polynucleotide

SEQ ID NO: 17 - DNA, 57 bp, Artificial Sequence
  Synthetic Polynucleotide
agctttttccaattgaaagagtcnccagcaatcattcatcacattcttctcatgttc

SEQ ID NO: 18 - DNA, 35 bp, Artificial Sequence
  Synthetic Polynucleotide
acatgagaagaatgtgatgaatgantactggagac

SEQ ID NO: 19 - DNA, 29 bp, Artificial Sequence
  Synthetic Polynucleotide
atcattcatcacattcttctcatgntctg

SEQ ID NO: 20 - DNA, 37 bp, Artificial Sequence
  Synthetic Polynucleotide
acatgagaagaatgtgatgaatgangccttaagactc

SEQ ID NO: 21 - DNA, 29 bp, Artificial Sequence
  Synthetic Polynucleotide
atcattcatcacattcttctcatgntctg

SEQ ID NO: 22 - DNA, 30 bp, Artificial Sequence; identical to SEQ ID NO: 14
  Synthetic Polynucleotide

SEQ ID NO: 23 - DNA, 51 bp, Artificial Sequence
  Synthetic Polynucleotide
agctttttccaattgaaagagtcngaaggcatcattcatcacattcttctc

SEQ ID NO: 24 - DNA, 30 bp, Artificial Sequence; identical to SEQ ID NO: 14
  Synthetic Polynucleotide

SEQ ID NO: 25 - DNA, 51 bp, Artificial Sequence
  Synthetic Polynucleotide
agctttttccaattgaaagagtcnataggcatcattcatcacattcttctc

SEQ ID NO: 26 - DNA, 46 bp, Artificial Sequence
  Synthetic Polynucleotide
acatgagaagaatgtgatgaatgangcctggagactcttgcaattg

SEQ ID NO: 27 - DNA, 29 bp, Artificial Sequence
  Synthetic Polynucleotide
atcattcatcacattcttctcatgntctg

SEQ ID NO: 28 - DNA, 30 bp, Artificial Sequence; identical to SEQ ID NO: 14
  Synthetic Polynucleotide

SEQ ID NO: 29 - DNA, 45 bp, Artificial Sequence
  Synthetic Polynucleotide
atgaatgatgcctggagacnccgtcaattggaaaaagctacaggg

SEQ ID NO: 30 - DNA, 44 bp, Artificial Sequence
  Synthetic Polynucleotide
acccattaaagatcgtaggaatcnctatgtgacatttcccatac

SEQ ID NO: 31 - DNA, 47 bp, Artificial Sequence
  Synthetic Polynucleotide
agattcctacgatctttaatgggnactattttgtaaagttgtttaag

SEQ ID NO: 32 - DNA, 53 bp, Artificial Sequence
  Synthetic Polynucleotide
acccattaaagatattaggaatctcntcgtgacatttcccatacctgaccttc

SEQ ID NO: 33 - DNA, 35 bp, Artificial Sequence
  Synthetic Polynucleotide
agagattcctaatatctttaatgggnactattttg

SEQ ID NO: 34 - DNA, 34 bp, Artificial Sequence
  Synthetic Polynucleotide
aaagggctgggttaatactctncagggtgggcag

SEQ ID NO: 35 - DNA, 30 bp, Artificial Sequence
  Synthetic Polynucleotide
aagagtattaacccagcccttngacttaag

SEQ ID NO: 36 - DNA, 45 bp, Artificial Sequence
  Synthetic Polynucleotide
aaagggctgggttaatactctnaggggtgggcagaaggaaggagc

SEQ ID NO: 37 - DNA, 30 bp, Artificial Sequence; identical to SEQ ID NO: 35
  Synthetic Polynucleotide

SEQ ID NO: 38 - DNA, 47 bp, Artificial Sequence
  Synthetic Polynucleotide
aaagggctgggttaatactctngttcaggggcagaaggaaggagccc

SEQ ID NO: 39 - DNA, 30 bp, Artificial Sequence; identical to SEQ ID NO: 35
  Synthetic Polynucleotide

SEQ ID NO: 40 - DNA, 50 bp, Artificial Sequence
  Synthetic Polynucleotide
aaagggctgggttaatactctngttggtcagcagaaggaaggagcccgag

SEQ ID NO: 41 - DNA, 30 bp, Artificial Sequence; identical to SEQ ID NO: 35
  Synthetic Polynucleotide

SEQ ID NO: 42 - DNA, 29 bp, Artificial Sequence
  Synthetic Polynucleotide
aaggagcccgaggttttangttttttatc

SEQ ID NO: 43 - DNA, 44 bp, Artificial Sequence
  Synthetic Polynucleotide
ataaaacctcgggctcctnccgcctgcccaccaacaagagtatt

SEQ ID NO: 44 - DNA, 31 bp, Artificial Sequence
  Synthetic Polynucleotide
atgttttttatcatgaatgtggactngaccg

SEQ ID NO: 45 - DNA, 34 bp, Artificial Sequence
  Synthetic Polynucleotide
aagtccacattcatgataaaaaacanaaaacctc

SEQ ID NO: 46 - DNA, 38 bp, Artificial Sequence
  Synthetic Polynucleotide
atgttttttatccagaatgtggactngaccgaggaagg

SEQ ID NO: 47 - DNA, 41 bp, Artificial Sequence
  Synthetic Polynucleotide
aagtccacattctggataaaaaacanaaaacctcgggctcc

SEQ ID NO: 48 - DNA, 45 bp, Artificial Sequence
  Synthetic Polynucleotide
atgtccgggttctgatagtttctaaancttttgaaggaaaaactg

SEQ ID NO: 49 - DNA, 40 bp, Artificial Sequence
  Synthetic Polynucleotide
atttagaaactatcagaacccggacantttctggtctgag

SEQ ID NO: 50 - DNA, 30 bp, Artificial Sequence
  Synthetic Polynucleotide
gattaactttattattaaaaattaaagagg

SEQ ID NO: 51 - DNA, 26 bp, Artificial Sequence
  Synthetic Polynucleotide
caacatgtaataatccttcctcggtc

SEQ ID NO: 52 - DNA, 22 bp, Artificial Sequence
  Synthetic Polynucleotide
gcatgaaggtcctggaagtctg

SEQ ID NO: 53 - DNA, 24 bp, Artificial Sequence
  Synthetic Polynucleotide
aggaagggttacatcatccagagc

SEQ ID NO: 54 - DNA, 21 bp, Artificial Sequence
  Synthetic Polynucleotide
ccatgtactacctccgcttgc

SEQ ID NO: 55 - DNA, 20 bp, Artificial Sequence
  Synthetic Polynucleotide
gcagatctcgagctcggatc

SEQ ID NO: 56 - DNA, 23 bp, Artificial Sequence
  Synthetic Polynucleotide
gcttatgtggaccccttgcactg

SEQ ID NO: 57 - DNA, 23 bp, Artificial Sequence
  Synthetic Polynucleotide
gaactgacagactcgccctatgt

SEQ ID NO: 58 - DNA, 19 bp, Artificial Sequence
  Synthetic Polynucleotide
gttgcaggcccagttgttg

SEQ ID NO: 59 - DNA, 22 bp, Artificial Sequence
  Synthetic Polynucleotide
gtgcagctgaacgtctgtctgt

SEQ ID NO: 60 - DNA, 18 bp, Artificial Sequence
  Synthetic Polynucleotide
tccggaggctggcattgt

SEQ ID NO: 61 - DNA, 21 bp, Artificial Sequence
  Synthetic Polynucleotide
ggtgtttactgggaaggcatc

SEQ ID NO: 62 - DNA, 20 bp, Artificial Sequence
  Synthetic Polynucleotide
caataatggggcactggctg

SEQ ID NO: 63 - DNA, 23 bp, Artificial Sequence
  Synthetic Polynucleotide
catgggcgcagcaggtgtatact

SEQ ID NO: 64 - DNA, 23 bp, Artificial Sequence
  Synthetic Polynucleotide
caaggtagatccgggacagacag

SEQ ID NO: 65 - DNA, 20 bp, Artificial Sequence
  Synthetic Polynucleotide
cctgctcatcagcaagatcc

SEQ ID NO: 66 - DNA, 20 bp, Artificial Sequence
  Synthetic Polynucleotide
tctcatccgtgttctcaacc

SEQ ID NO: 67 - DNA, 25 bp, Artificial Sequence
  Synthetic Polynucleotide
catccgtaaagacctctatgccaac

SEQ ID NO: 68 - DNA, 19 bp, Artificial Sequence
  Synthetic Polynucleotide
atggagccaccgatccaca

SEQ ID NO: 69 - DNA, 75 bp, Artificial Sequence
  Synthetic Polynucleotide
aatgatacggcgaccaccgagatctacactctttccctacacgacgctcttccgatctnnnnnnnccctgtacac

SEQ ID NO: 70 - DNA, 44 bp, Artificial Sequence
  Synthetic Polynucleotide
caagcagaagacggcatacgagctcttccgatctgagtgggatg